(19) United States
(12) Patent Application Publication
(10) Pub. No.: US 2026/0327244 A1
(43) Pub. Date: Oct. 22, 2026

(54) PYRIDO[4,3-D]PYRIMIDINE COMPOUNDS

(71) Applicant: Pfizer Inc., New York, NY (US)

(72) Inventors: Jacob Cole DEFOREST, San Diego, CA (US); Asako NAGATA, San Diego, CA (US); Simon Paul PLANKEN, San Marcos, CA (US); Jillian Elyse SPANGLER, San Diego, CA (US); Scott Channing SUTTON, San Diego, CA (US); John Howard TATLOCK, San Diego, CA (US)

(73) Assignee: Pfizer Inc., New York, NY (US)

(21) Appl. No.: 18/880,953

(22) PCT Filed: Jun. 30, 2023
(86) PCT No.: PCT/IB2023/056844

§ 371 Date: Jan. 3, 2025

Related U.S. Application Data
(60) Provisional application No. 63/358,324, filed on Jul. 5, 2022.
(60) Provisional application No. 63/394,338, filed on Aug. 2, 2022.
(60) Provisional application No. 63/414,939, filed on Oct. 11, 2022.
(60) Provisional application No. 63/502,931, filed on May 18, 2023.

(51) Int. Cl. C07D 519/00; A61K 31/553; A61K 45/06; A61P 35/00

(52) U.S. Cl. CPC C07D 519/00; A61K 31/553; A61K 45/06; A61P 35/00

(57) ABSTRACT

The invention relates to compounds of Formula (I)-(VII), or pharmaceutically acceptable salts thereof, to their use in medicine; to compositions containing them; to processes for their preparation; and to intermediates used in such processes. The compounds the present invention may be useful in the treatment, prevention, suppression and amelioration of cancers, diseases, or disorders. Formula (V), (VI) & (VII)

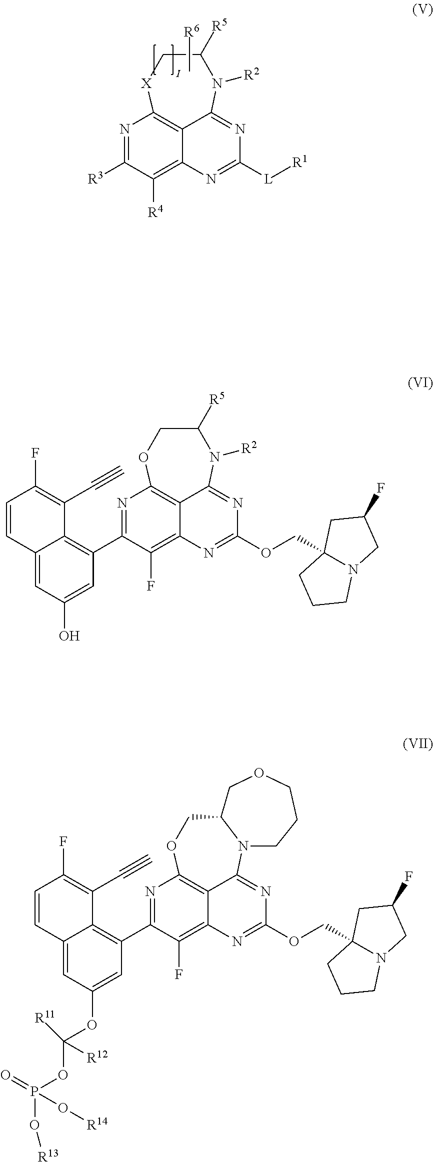

### BACKGROUND OF THE INVENTION

The present invention relates to novel pyrido[4,3-d]pyrimidine compounds as Kirsten rat sarcoma viral oncogene homolog (KRAS) Inhibitors. The invention also relates to the preparation of the compounds and intermediates used in the preparation, compositions containing the compounds, and uses of the compounds for the treatment of KRAS related diseases such as cancers.

KRAS, HRAS (Harvey Rat sarcoma virus) and NRAS (Neuroblastoma RAS Viral Oncogene Homolog) belong to a group of GTPases that are critical in the survival and proliferation of cells through complex signaling cascades. Mutated RAS genes are found in approximately 30% of all cancers (Hyun et al 2021 Int. J. Mol. Sci. 22 (22), 12142). KRAS is the most frequently mutated RAS isoform in cancer cells (up to 85%), leading to development of cancers including non-small cell lung cancer (NSCLC), colorectal and pancreatic cancer that collectively and individually have significant unmet medical needs for affected patients. KRAS mutations are seen extensively in pancreatic ductal adenocarcinoma (PDAC). Mutations in KRAS have been observed in 30% of NSCLC cases, which is the major (80%) form of lung cancer. KRAS mutations seen in NSCLC include 39% of G12C, 18-21% of G12V, and 17-18% of G12D. KRAS mutations occur in 35-45% of colon cancers, leading to drug resistance.

Inhibitors of KRAS have been sought for decades, with recent advances seeing approval of sotorasib and subsequent KRAS G12C targeting compounds in trials (Palmer et al 2021 NPJ Precision Oncology, 5, 98). Sotorasib specifically targets mutations in KRAS through covalent modification of mutant cysteine at position 12. For this reason, sotorasib and other currently known KRAS inhibitors that rely on the same mechanism of action may be narrow in treatment scope and be of limited use when considering other major KRAS mutations such as G12V and G12D.

Accordingly, there remains a need for new KRAS inhibitors that may be used for the treatment of a broader scope of cancers.

### SUMMARY OF THE INVENTION

The present invention provides, in part, compounds of Formula (I) to Formula (VII), pharmaceutically acceptable salts thereof. The compounds of the present invention may inhibit the activities of all KRAS G12C, KRAS G12D, and KRAS G12V receptors, and may be useful in the treatment, prevention, suppression, and amelioration of diseases such as cancers, disorders and conditions mediated by any of KRAS G12C, KRAS G12D, and KRAS G12V receptors, or a combination thereof. Also provided are pharmaceutical compositions, comprising the compounds or salts of the invention, alone or in combination with additional anticancer therapeutic agents. The present invention also provides, in part, methods for preparing such compounds, pharmaceutically acceptable salts and compositions of the invention, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the invention there is provided a compound of Formula (I)

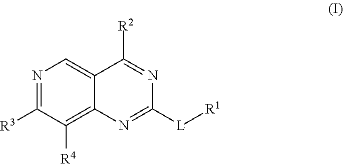

or a pharmaceutically acceptable salt thereof, wherein:
R¹ is C₃-C₁₀ cycloalkyl or 4-12 membered heterocycloalkyl comprising one, two or three heteroatoms selected from the group consisting of N, O, and S, wherein the C₃-C₁₀ cycloalkyl or the 4-12 membered heterocycloalkyl is each optionally substituted with one, two or three substituents independently selected from the group consisting of —OH, —CN, halogen, C₁-C₃ alkyl, C₁-C₃ fluoroalkyl, and C₁-C₃ alkoxy; R² is selected from the group consisting of:

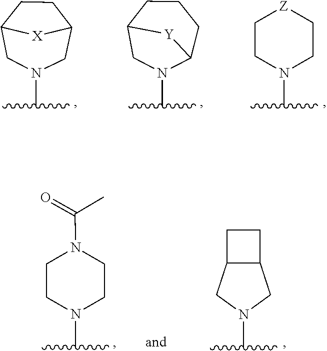

wherein R² is optionally substituted with one, two or three substituents independently selected from the group consisting of —OH, —CH₂OH, —CN, —CH₂CN, halogen, C₁-C₃ alkyl, C₁-C₃ fluoroalkyl, and C₁-C₃ alkoxy;
R³ is C₆-C₁₀ aryl or 4-12 membered heteroaryl comprising one, two, three, or four N atoms, wherein the C₆-C₁₀ aryl or the 4-12 membered heteroaryl is optionally substituted with one, two, three or four substituents independently selected from the group consisting of —OH, halogen, —CN, C₁-C₃ alkyl, C₁-C₃ fluoroalkyl, C₁-C₃ alkoxy, and C₂-C₃ alkynyl;
R⁴ is hydrogen, halogen, C₁-C₃ alkyl, or C₁-C₃ fluoroalkyl;
L is a linker comprising one, two or three members independently selected from the group consisting of —O—, —S—, —NRS—, and —CR⁶R⁷—;
R⁵, R⁶, and R⁷ are each independently H or C₁-C₃ alkyl;
X and Y are each independently selected from the group consisting of O, S, —SO₂—, and C₁-C₂ alkylene; and
Z is a bond, C₁-C₂ alkylene, O, S, or —SO₂—,
wherein when Z is O, R² is substituted with one, two or three substituents independently selected from the group consisting of —OH, —CH₂OH, —CN, —CH₂CN, halogen, C₁-C₃ alkyl, C₁-C₃ fluoroalkyl, and C₁-C₃ alkoxy,
wherein when R² is

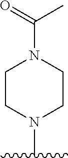 [SMILES: *N1CCN(C(C)=O)CC1]

that comprises a piperazinyl ring, the piperazinyl ring is substituted with one, two or three substituents independently selected from the group consisting of —OH, —CH₂OH, —CN, —CH₂CN, halogen, C₁-C₃ alkyl, C₁-C₃ fluoroalkyl, and C₁-C₃ alkoxy.

Described below are embodiments of the invention, where for convenience Embodiment 1 (E1) is identical to the embodiment of Formula (I) provided above.

It is to be understood that both the foregoing general description and the following detailed description are exemplary and explanatory only and are not restrictive of the invention, as claimed.

### BRIEF DESCRIPTION OF THE DRAWINGS

FIG. 1 provides mouse PK study results showing unbound plasma concentrations of Example 33 in female NSG mouse plasma after oral dosing of female NSG Mice at 100 mg/kg (n=3, mean+/−SD). The solution of Example 33 was prepared using a Pluronic-based formulation [2.5% (w/v) Pluronic F-68 (Poloxamer 188)], and the suspension arms of both Example 33 and Example 34 were prepared using a 0.5% (w/v) methylcellulose formulation.

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.
E1 A compound of Formula (I) or a pharmaceutically acceptable salt thereof, as defined above.
E2 A compound of Formula (I-a)

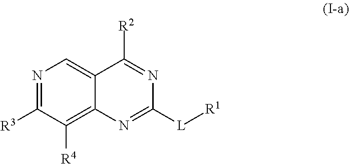

or a pharmaceutically acceptable salt thereof, wherein:
R¹ is C₃-C₁₀ cycloalkyl or 4-12 membered heterocycloalkyl comprising one, two or three heteroatoms selected from the group consisting of N, O, and S, wherein the C₃-C₁₀ cycloalkyl or the 4-12 membered heterocycloalkyl is each optionally substituted with one, two or three substituents independently selected from the group consisting of —OH, —CN, halogen, C₁-C₃ alkyl, C₁-C₃ fluoroalkyl, and C₁-C₃ alkoxy;
R² is a 4-12 membered heterocycloalkyl comprising one, two or three heteroatoms selected from the group consisting of N, O, and S, wherein the 4-12 membered heterocycloalkyl has one and only one nitrogen as a ring member that is directly attached to the pyrido[4,3-d]pyrimidine core of Formula (I-a), wherein the 4-12 membered heterocycloalkyl is optionally substituted with one, two or three substituents independently selected from the group consisting of —OH, —CH₂OH, —CN, —CH₂CN, halogen, C₁-C₃ alkyl, C₁-C₃ fluoroalkyl, and C₁-C₃ alkoxy, and when R² is a morpholinyl ring, the morpholinyl ring is substituted with one, two or three substituents independently selected from the group consisting of —OH, —CH₂OH, —CN, —CH₂CN, halogen, C₁-C₃ alkyl, C₁-C₃ fluoroalkyl, and C₁-C₃ alkoxy;
R³ is C₆-C₁₀ aryl or 4-12 membered heteroaryl comprising one, two, three, or four N atoms, wherein the C₆-C₁₀ aryl or the 4-12 membered heteroaryl is optionally substituted with one, two, three or four substituents independently selected from the group consisting of —OH, halogen, —CN, C₁-C₃ alkyl, C₁-C₃ fluoroalkyl, C₁-C₃ alkoxy, and C₂-C₃ alkynyl
R⁴ is hydrogen, halogen, or C₁-C₃ alkyl;
L is a linker comprising one, two or three members independently selected from the group consisting of —O—, —S—, —NRS—, and —CR⁶R⁷—; and
R⁵, R⁶, and R⁷ are each independently H or C₁-C₃ alkyl.
E3 A compound of embodiment E1 or embodiment E2, or a pharmaceutically acceptable salt thereof, wherein L is —O—CH₂—.
E4 A compound of any one of embodiments E1 to E3, or a pharmaceutically acceptable salt thereof, wherein R¹ is 5-8 membered heterocycloalkyl comprising one N as the sole heteroatom, and the 5-8 membered heterocycloalkyl is optionally substituted with one, two or three substituents independently selected from the group consisting of halogen and C₁-C₃ alkyl.
E5 A compound of any one of embodiments E1 to E4, or a pharmaceutically acceptable salt thereof, wherein R¹ is selected from the group consisting of:

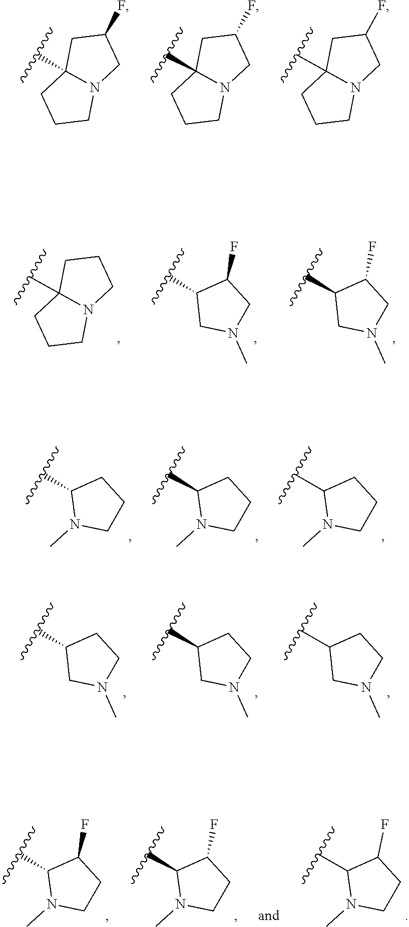

E6 A compound of embodiment E5, or a pharmaceutically acceptable salt thereof, wherein R¹ is selected from the group consisting of:

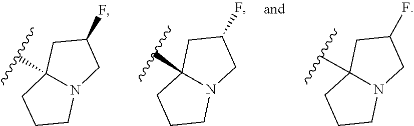

E7 A compound of any one of embodiments E1 to E6, or a pharmaceutically acceptable salt thereof, wherein R² is selected from the group consisting of:

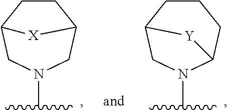

wherein X and Y are each independently O or —CH₂—, and R² is optionally substituted with one, two, or three substituents independently selected from the group consisting of —OH and —CN.
E8 A compound of embodiment E7, or a pharmaceutically acceptable salt thereof, wherein R² is selected from the group consisting of:

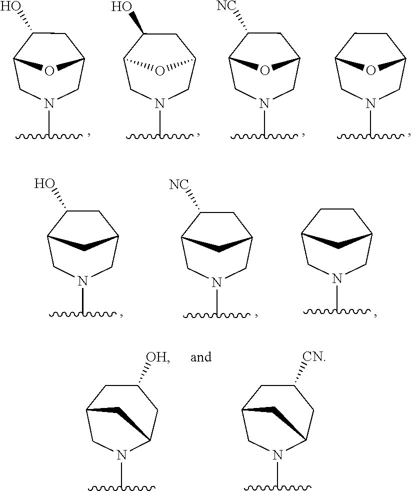

E9 A compound of embodiment E8, or a pharmaceutically acceptable salt thereof, wherein R² is selected from the group consisting of:

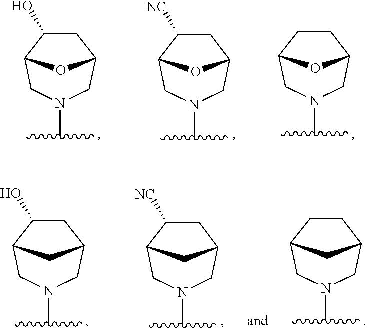 [SMILES: *N1C[C@H]2C[C@@H](O)[C@@H](C1)O2 ; *N1C[C@H]2C[C@@H](C#N)[C@@H](C1)O2 ; *N1C[C@H]2CC[C@@H](C1)O2 ; *N1C[C@@H]2C[C@H](C1)[C@H](O)C2 ; *N1C[C@@H]2C[C@H](C1)[C@H](C#N)C2 ; *N1C[C@@H]2CC[C@@H](C2)C1]

E10 A compound of any one of embodiments E1 to E6, or a pharmaceutically acceptable salt thereof, wherein R² is

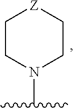

wherein Z is a bond, —CH₂—, or O, wherein R² is optionally substituted with one, two or three substituents independently selected from the group consisting of —OH, —CH₂OH, —CN, —CH₂CN, halogen, C₁-C₃ alkyl, C₁-C₃ fluoroalkyl, and C₁-C₃ alkoxy, and when Z is O, R² is substituted with one, two or three substituents independently selected from the group consisting of —OH, —CH₂OH, —CN, —CH₂CN, halogen, C₁-C₃ alkyl, C₁-C₃ fluoroalkyl, and C₁-C₃ alkoxy.
E11 A compound of embodiment E10, or a pharmaceutically acceptable salt thereof, wherein R² is selected from the group consisting of:

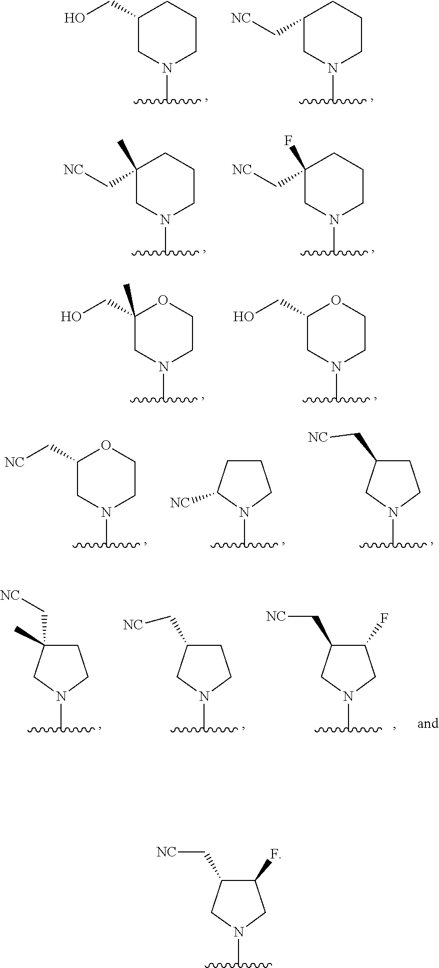

E12 A compound of any one of embodiments E1 to E6, or a pharmaceutically acceptable salt thereof, wherein R² is selected from the group consisting of:

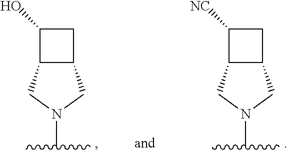 [SMILES: *N1C[C@H]2C[C@@H](O)[C@H]2C1 ; *N1C[C@H]2C[C@@H](C#N)[C@H]2C1]

E13 A compound of any one of embodiments E1 to E12, or a pharmaceutically acceptable salt thereof, wherein R³ is a C₆-C₁₀ bicyclic aryl or a 4-12 membered bicyclic heteroaryl, and wherein R³ is optionally substituted with one, two, three or four substituents independently selected from the group consisting of —OH, halogen, —CN, C₁-C₃ alkyl, C₁-C₃ fluoroalkyl, and C₂-C₃ alkynyl.
E14 A compound of embodiment E13, or a pharmaceutically acceptable salt thereof, wherein R³ is naphthyl optionally substituted with one, two, three or four substituents independently selected from the group consisting of —OH, halogen, —CN, C₁-C₃ alkyl, C₁-C₃ fluoroalkyl, and C₂-C₃ alkynyl.
E15 A compound of embodiment E14, or a pharmaceutically acceptable salt thereof, wherein R³ is selected from the group consisting of:

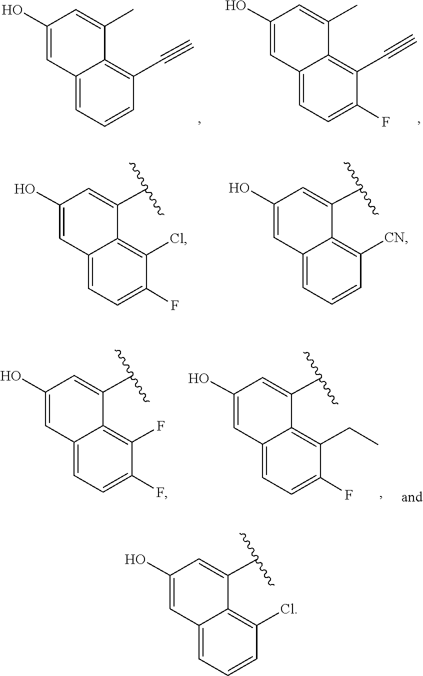

E16 A compound of any one of embodiments E1 to E15, or a pharmaceutically acceptable salt thereof, wherein R⁴ is Cl or F.
E17 A compound of any one of embodiments E1-E16, or a pharmaceutically acceptable salt thereof, wherein the compound is selected from the group consisting of:

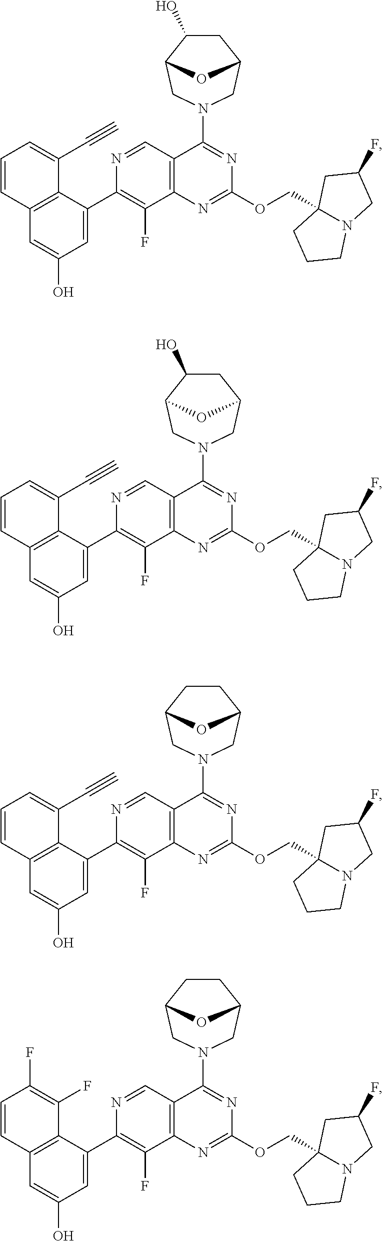 [SMILES: C#Cc1cccc2cc(O)cc(-c3ncc4c(N5C[C@H]6C[C@@H](O)[C@@H](C5)O6)nc(OC[C@@]56CCCN5C[C@H](F)C6)nc4c3F)c12 ; C#Cc1cccc2cc(O)cc(-c3ncc4c(N5C[C@@H]6C[C@H](O)[C@H](C5)O6)nc(OC[C@@]56CCCN5C[C@H](F)C6)nc4c3F)c12 ; C#Cc1cccc2cc(O)cc(-c3ncc4c(N5C[C@H]6CC[C@@H](C5)O6)nc(OC[C@@]56CCCN5C[C@H](F)C6)nc4c3F)c12 ; Oc1cc(-c2ncc3c(N4C[C@H]5CC[C@@H](C4)O5)nc(OC[C@@]45CCCN4C[C@H](F)C5)nc3c2F)c2c(F)c(F)ccc2c1]

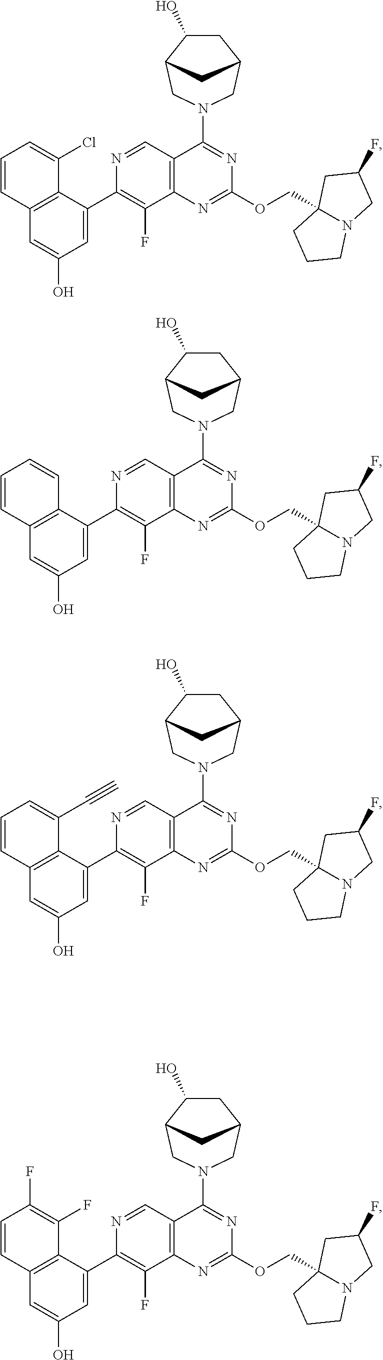 [SMILES: Oc1cc(-c2ncc3c(N4C[C@@H]5C[C@H](C4)[C@H](O)C5)nc(OC[C@@]45CCCN4C[C@H](F)C5)nc3c2F)c2c(Cl)cccc2c1 ; Oc1cc(-c2ncc3c(N4C[C@@H]5C[C@H](C4)[C@H](O)C5)nc(OC[C@@]45CCCN4C[C@H](F)C5)nc3c2F)c2ccccc2c1 ; C#Cc1cccc2cc(O)cc(-c3ncc4c(N5C[C@@H]6C[C@H](C5)[C@H](O)C6)nc(OC[C@@]56CCCN5C[C@H](F)C6)nc4c3F)c12 ; Oc1cc(-c2ncc3c(N4C[C@@H]5C[C@H](C4)[C@H](O)C5)nc(OC[C@@]45CCCN4C[C@H](F)C5)nc3c2F)c2c(F)c(F)ccc2c1]

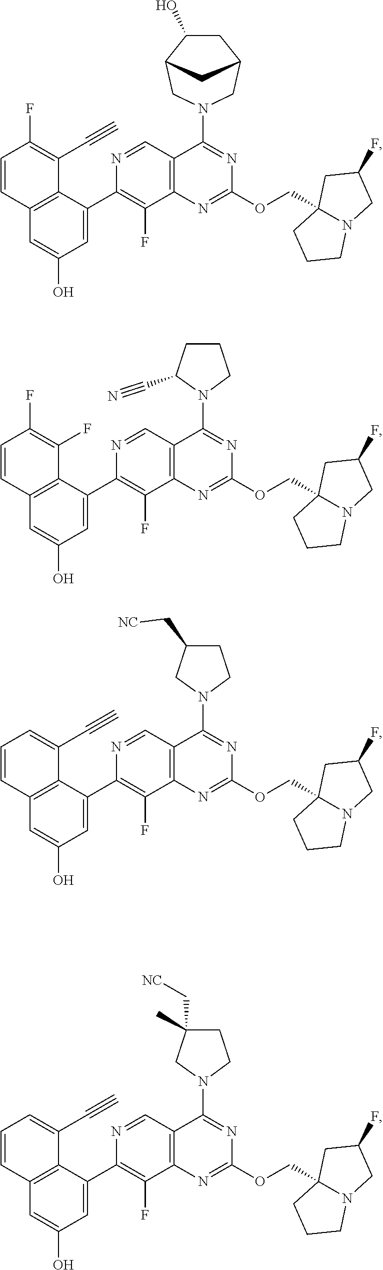 [SMILES: C#Cc1c(F)ccc2cc(O)cc(-c3ncc4c(N5C[C@@H]6C[C@H](C5)[C@H](O)C6)nc(OC[C@@]56CCCN5C[C@H](F)C6)nc4c3F)c12 ; N#C[C@@H]1CCCN1c1nc(OC[C@@]23CCCN2C[C@H](F)C3)nc2c(F)c(-c3cc(O)cc4ccc(F)c(F)c34)ncc12 ; C#Cc1cccc2cc(O)cc(-c3ncc4c(N5CC[C@H](CC#N)C5)nc(OC[C@@]56CCCN5C[C@H](F)C6)nc4c3F)c12 ; C#Cc1cccc2cc(O)cc(-c3ncc4c(N5CC[C@@](C)(CC#N)C5)nc(OC[C@@]56CCCN5C[C@H](F)C6)nc4c3F)c12]

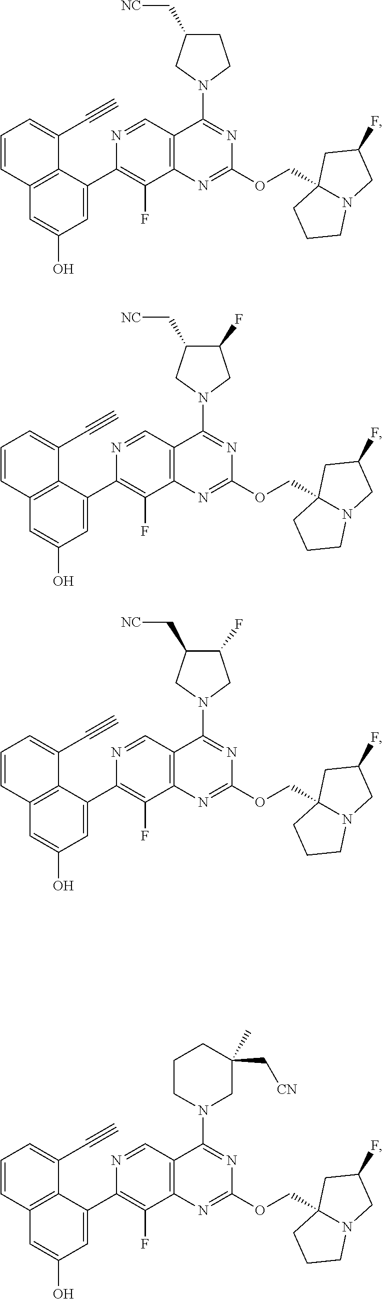 [SMILES: C#Cc1cccc2cc(O)cc(-c3ncc4c(N5CC[C@@H](CC#N)C5)nc(OC[C@@]56CCCN5C[C@H](F)C6)nc4c3F)c12 ; C#Cc1cccc2cc(O)cc(-c3ncc4c(N5C[C@H](CC#N)[C@@H](F)C5)nc(OC[C@@]56CCCN5C[C@H](F)C6)nc4c3F)c12 ; C#Cc1cccc2cc(O)cc(-c3ncc4c(N5C[C@@H](CC#N)[C@H](F)C5)nc(OC[C@@]56CCCN5C[C@H](F)C6)nc4c3F)c12 ; C#Cc1cccc2cc(O)cc(-c3ncc4c(N5CCC[C@@](C)(CC#N)C5)nc(OC[C@@]56CCCN5C[C@H](F)C6)nc4c3F)c12]

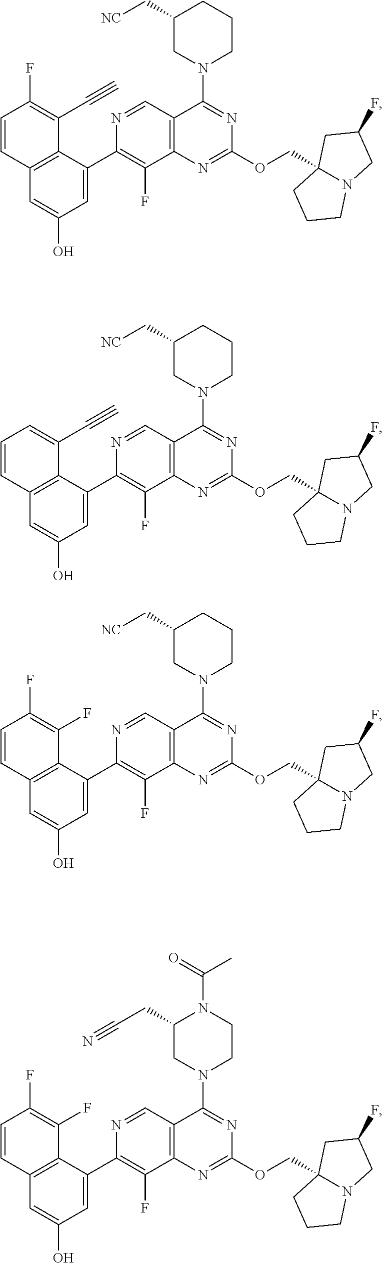 [SMILES: C#Cc1c(F)ccc2cc(O)cc(-c3ncc4c(N5CCC[C@@H](CC#N)C5)nc(OC[C@@]56CCCN5C[C@H](F)C6)nc4c3F)c12 ; C#Cc1cccc2cc(O)cc(-c3ncc4c(N5CCC[C@@H](CC#N)C5)nc(OC[C@@]56CCCN5C[C@H](F)C6)nc4c3F)c12 ; N#CC[C@@H]1CCCN(c2nc(OC[C@@]34CCCN3C[C@H](F)C4)nc3c(F)c(-c4cc(O)cc5ccc(F)c(F)c45)ncc23)C1 ; CC(=O)N1CCN(c2nc(OC[C@@]34CCCN3C[C@H](F)C4)nc3c(F)c(-c4cc(O)cc5ccc(F)c(F)c45)ncc23)C[C@@H]1CC#N]

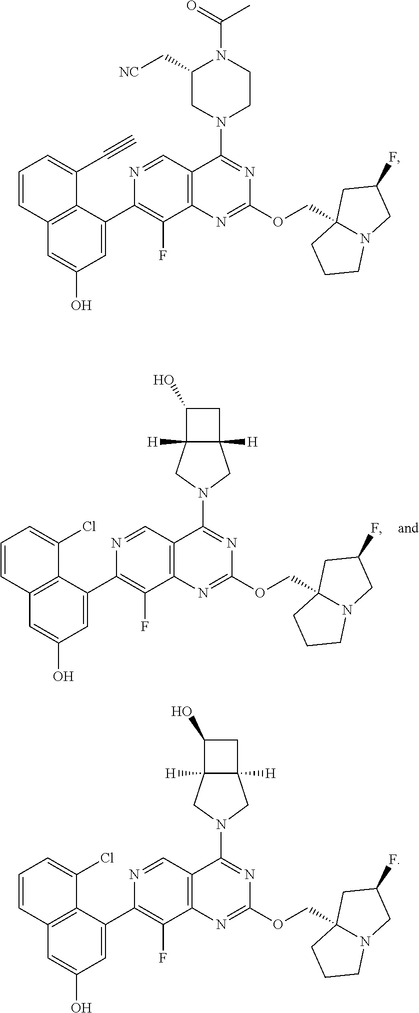 [SMILES: C#Cc1cccc2cc(O)cc(-c3ncc4c(N5CCN(C(C)=O)[C@@H](CC#N)C5)nc(OC[C@@]56CCCN5C[C@H](F)C6)nc4c3F)c12 ; Oc1cc(-c2ncc3c(N4C[C@H]5C[C@@H](O)[C@H]5C4)nc(OC[C@@]45CCCN4C[C@H](F)C5)nc3c2F)c2c(Cl)cccc2c1 ; Oc1cc(-c2ncc3c(N4C[C@@H]5C[C@H](O)[C@@H]5C4)nc(OC[C@@]45CCCN4C[C@H](F)C5)nc3c2F)c2c(Cl)cccc2c1]

E18 A compound of embodiment E17, wherein the compound is selected from the group consisting of:

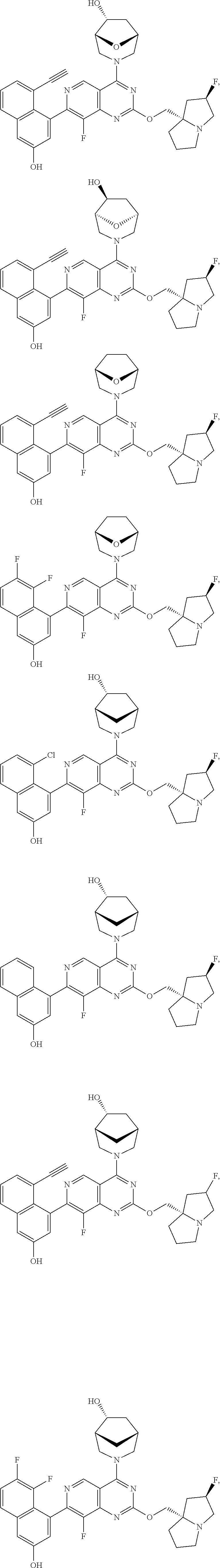

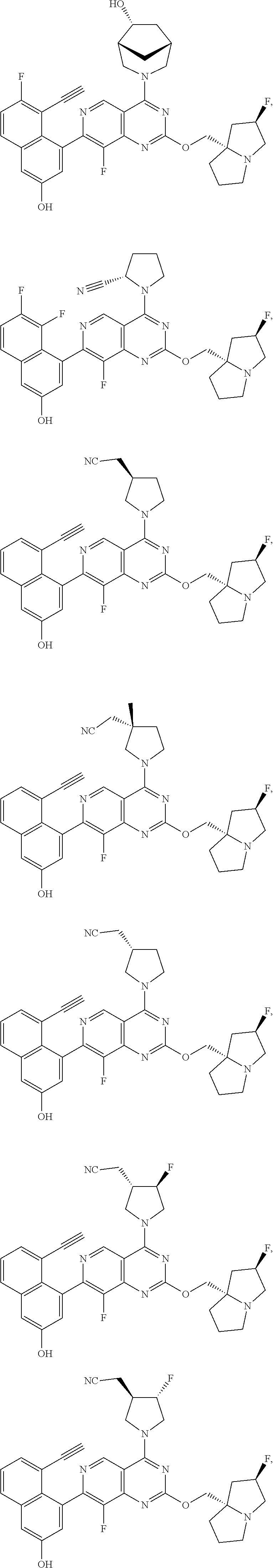

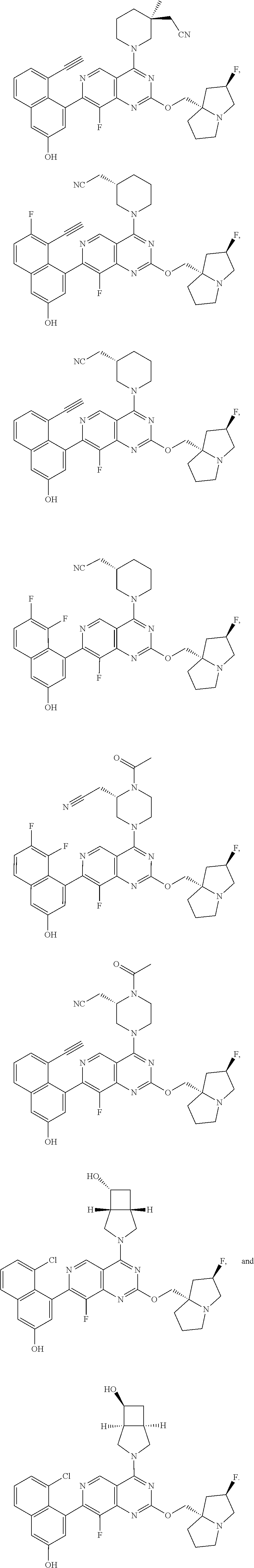

E19 A pharmaceutical composition comprising a compound of any one of embodiments E1 to E18, or a pharmaceutically acceptable salt thereof, and at least one pharmaceutically acceptable excipient.
E20 A method for treating cancer, comprising administering to a subject in need thereof a therapeutically effective amount of a compound of any one of embodiments E1 to E18, or a pharmaceutically acceptable salt thereof.
E21 A method for treating cancer, comprising administering to a subject in need thereof a therapeutically effective amount of a compound of any one of embodiments E1 to E18, or a pharmaceutically acceptable salt thereof as a single agent.
E22 A method for treating cancer, comprising administering to a subject in need thereof a therapeutically effective amount of a compound of any one of embodiments E1 to E18, or a pharmaceutically acceptable salt thereof, and further comprising administering a therapeutically effective amount of an additional anticancer therapeutic agent.
E23 A method for treating cancer of any one embodiments E20 to E22, wherein the cancer is small cell lung cancer (NSCLC), pancreatic cancer, or colorectal cancer.
E24 A compound of any one of embodiments E1 to E18, or a pharmaceutically acceptable salt thereof, for use as a medicament.
E25 A compound of any one of embodiments E1 to E18, or a pharmaceutically acceptable salt thereof, for use in the treatment of cancer.
E26 A compound for use in the treatment of cancer according to embodiment E25, wherein the cancer is small cell lung cancer (NSCLC), pancreatic cancer, or colorectal cancer.
E27 Use of a compound of any one of embodiments E1 to E18, or a pharmaceutically acceptable salt thereof, for the manufacture of a medicament for the treatment of cancer.
E28 Use of a compound, or a pharmaceutically acceptable salt thereof, for the manufacture of a medicament for the treatment of cancer according to embodiment E27, wherein the cancer is small cell lung cancer (NSCLC), pancreatic cancer, or colorectal cancer.
E29 A method for the treatment of a disorder mediated by inhibition of KRAS G12C, KRAS G12D, and KRAS G12V receptors in a subject, comprising administering to the subject in need thereof a compound of any one of embodiments E1 to E18, or a pharmaceutically acceptable salt thereof, in an amount that is effective for treating the disorder.
E30 A pharmaceutical combination comprising a compound of any one of embodiments E1 to E18, or a pharmaceutically acceptable salt thereof, and at least one additional therapeutic agent or a pharmaceutically acceptable salt thereof.
E31 A pharmaceutical composition comprising the pharmaceutical combination of embodiment E30 and at least one excipient.
E32 A compound of Formula (II):

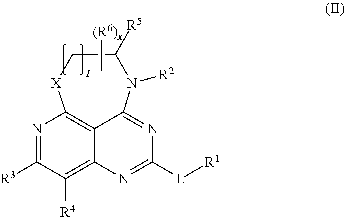

or a pharmaceutically acceptable salt thereof, wherein:
R¹ is C₃-C₁₀ cycloalkyl or 4-12 membered heterocycloalkyl comprising one, two or three heteroatoms selected from the group consisting of N, O, and S, each optionally substituted with one, two or three substituents independently selected from the group consisting of —OH, —CN, halogen, C₁-C₃ alkyl, C₁-C₃ fluoroalkyl, and C₁-C₃ alkoxy;
R² is H or is selected from the group consisting of —(C₁-C₆ alkylene)-OH, —(C₁-C₆ alkylene)-CN, —(C₁-C₆ alkylene)-SH, —(C₁-C₃ alkylene)-S—(C₁-C₃ alkyl), —(C₁-C₃ alkylene)-(S═O)—(C₁-C₃ alkyl), —(C₁-C₃ alkylene)-(SO₂)—(C₁-C₃ alkyl), C₁-C₆ alkyl, C₃-C₆ cycloalkyl, C₁-C₆ fluoroalkyl, C₃-C₆ fluorocycloalkyl, and C₁-C₆ alkoxy, each optionally substituted with one, two or three substituents independently selected from the group consisting of —OH, —CN, —NH₂, —SH, —(C₁-C₄ alkylene)-CN, —(C₁-C₄ alkylene)-OH, halogen, C₁-C₃ alkyl, C₃-C₆ cycloalkyl, C₁-C₃ fluoroalkyl, and C₁-C₃ alkoxy;
R³ is C₆-C₁₀ aryl or 4-12 membered heteroaryl comprising one, two, three or four N atoms, each optionally substituted with one, two, three or four substituents independently selected from the group consisting of —OH, halogen, —CN, C₁-C₃ alkyl, C₁-C₃ fluoroalkyl, C₁-C₃ alkoxy, and C₂-C₃ alkynyl, when R³ is substituted with two C₁-C₃ alkyl groups, the two C₁-C₃ alkyl groups can join to form a 3-6 membered ring fused to said C₆-C₁₀ aryl or 4-12 membered heteroaryl;
R⁴ is H, halogen, C₁-C₃ alkyl, or C₁-C₃ fluoroalkyl;
R⁵ is H, —OH, halogen, —NH₂, CN, or selected from the group consisting of —(C₁-C₆ alkylene)-OH, —(C₁-C₆ alkylene)-CN, —(C₁-C₆ alkylene)-SH, —(C₁-C₃ alkylene)-S—(C₁-C₃ alkyl), —(C₁-C₃ alkylene)-(S═O)—(C₁-C₃ alkyl), —(C₁-C₃ alkylene)-(SO₂)—(C₁-C₃ alkyl), C₁-C₆ alkyl, C₃-C₆ cycloalkyl, C₁-C₆ fluoroalkyl, C₃-C₆ fluorocycloalkyl, and C₁-C₆ alkoxy each is optionally substituted with one, two or three substituents independently selected from the group consisting of —OH, —CN, —NH₂, —SH, —(C₁-C₄ alkylene)-CN, halogen, C₁-C₃ alkyl, C₃-C₆ cycloalkyl, C₁-C₃ fluoroalkyl, and C₁-C₃ alkoxy; or alternatively, R⁵ and the carbon that R⁵ is attached to, and R² and the nitrogen that R² is attached to together to form a 4-8 membered heterocycloalkyl comprising one, two or three heteroatoms selected from the group consisting of N, O, and S, or heteroatom-containing groups selected from the group consisting of N(C₁-C₆ alkyl), —(S═O)—, and —(SO₂)—, wherein said 4-8 membered heterocycloalkyl is optionally substituted with one, two or three substituent groups selected from the group consisting of —OH, —CN, halogen, C₁-C₃ alkyl, —(C₁-C₆ alkylene)-CN, and —(C₁-C₆ alkylene)-OH;
R⁶ at each occurrence is independently H, —OH, halogen, CN, or selected from the group consisting of —(C₁-C₆ alkylene)-OH, —(C₁-C₆ alkylene)-CN, C₁-C₆ alkyl, C₃-C₆ cycloalkyl, C₁-C₆ fluoroalkyl, C₃-C₆ fluorocycloalkyl, and C₁-C₆ alkoxy, each optionally substituted with one, two or three substituents independently selected from the group consisting of —OH, —CN, —(C₁-C₄ alkylene)-CN, halogen, C₁-C₃ alkyl, C₃-C₆ cycloalkyl, C₁-C₃ fluoroalkyl, and C₁-C₃ alkoxy;
L is a linker comprising one, two or three members independently selected from the group consisting of —O—, —S—, —NR⁷—, and —CR⁸R⁹—;
R⁷, R⁸, and R⁹ are each independently H or C₁-C₃ alkyl;
X is O, N, or S;
l is 1 or 2; and
x is 1 or 2.
E33 A compound of embodiment E32, or a pharmaceutically acceptable salt thereof, wherein the linker L is —(O—CH₂)—.
E34 A compound of embodiment E32 or embodiment E33, or a pharmaceutically acceptable salt thereof, wherein R¹ is 5-8 membered heterocycloalkyl comprising one N as the sole heteroatom, and said 5-8 membered heterocycloalkyl is optionally substituted with one, two or three substituents independently selected from the group consisting of halogen, and C₁-C₃ alkyl.
E35 A compound of embodiment E34, or a pharmaceutically acceptable salt thereof, wherein R¹ is selected from the group consisting of:

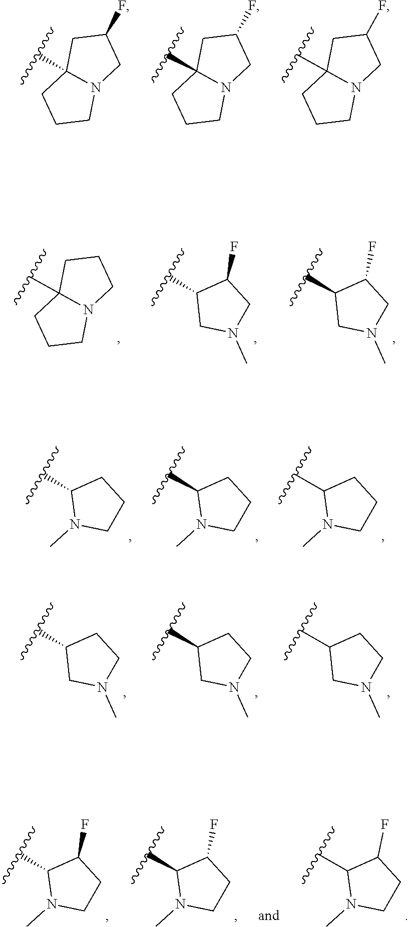

E36 A compound of embodiment E35, or a pharmaceutically acceptable salt thereof, wherein R¹ is selected from the group consisting of:

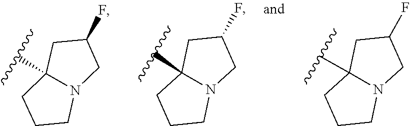

E37 A compound of any one of embodiments E32 to E36, or a pharmaceutically acceptable salt thereof, wherein R² is H or is selected from the group consisting of —(C₁-C₅ alkylene)-OH and C₁-C₅ alkyl, each optionally substituted with one, two or three substituents independently selected from the group consisting of —OH, —CN, and halogen.
E38 A compound of any one of embodiments E32 to E37, or a pharmaceutically acceptable salt thereof, wherein R⁵ and R⁶ are each H, —OH, —CN, halogen, or independently selected from the group consisting —(C₁-C₅ alkylene)-OH,] and C₁-C₅ alkyl, each optionally substituted with one, two or three substituents independently selected from the group consisting of —OH, —CN, and halogen.
E39 A compound of any one of embodiments E32 to E38, wherein the compound has Formula (III):

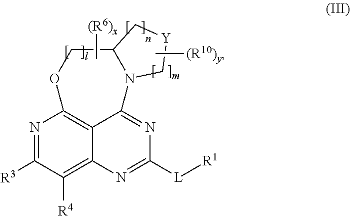

wherein Y is selected from the group consisting of CH₂, O, N(C₁-C₆ alkyl), S, (S═O), and (SO₂);
R¹⁰ at each occurrence is independently selected from the group consisting of —OH, —CN, halogen, C₁-C₃ alkyl, —(C₁-C₆ alkylene)-CN, and —(C₁-C₆ alkylene)-OH; and
m and n are each independently 0, 1, 2 or 3, y is 1, 2, or 3, and m plus n is 1, 2, 3, 4, or 5.
E40 A compound of embodiment E39, wherein Y is —CH₂— or O.
E41 A compound of any one of embodiments E32 to E40, or a pharmaceutically acceptable salt thereof, wherein R³ is a bicyclic aryl or a bicyclic heteroaryl, each optionally substituted with one to four substituents independently selected from the group consisting of —OH, halogen, —CN, C₁-C₃ alkyl, C₁-C₃ fluoroalkyl, and C₂-C₃ alkynyl, when R³ is substituted with two C₁-C₃ alkyl groups, the two C₁-C₃ alkyl groups can join to form a 3-6 membered ring.
E42 A compound of embodiment E41, or a pharmaceutically acceptable salt thereof, wherein R³ is a naphthyl optionally substituted with one to four substituents independently selected from the group consisting of —OH, halogen, —CN, C₁-C₃ alkyl, C₁-C₃ fluoroalkyl, and C₂-C₃ alkynyl.
E43 A compound of embodiment E42, or a pharmaceutically acceptable salt thereof, wherein R³ is selected from the group consisting of:

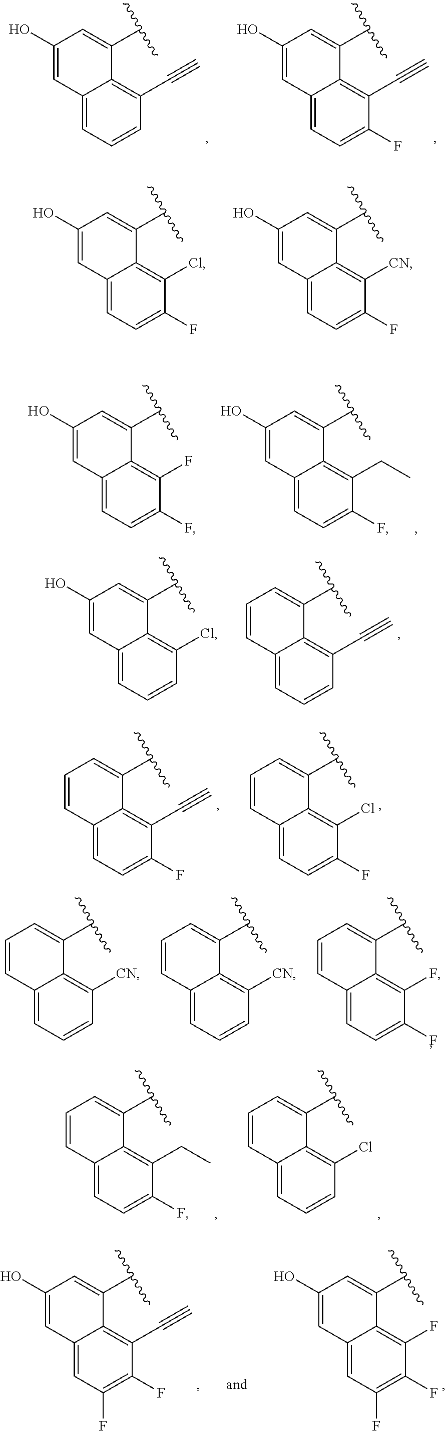

E44 A compound of any one of embodiments E32 to E43, or a pharmaceutically acceptable salt thereof, wherein R⁴ is Cl or F.
E45 A compound of embodiment E32, or a pharmaceutically acceptable salt thereof, wherein the compound has Formula (IV):

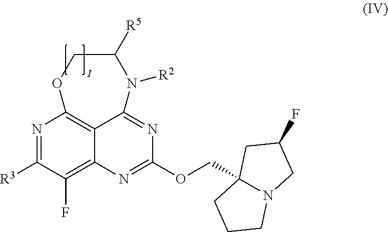

wherein R³ is a naphthyl optionally substituted with one to four substituents independently selected from the group consisting of —OH, halogen, —CN, C₁-C₃ alkyl, C₁-C₃ fluoroalkyl, and C₂-C₃ alkynyl; l is 1 or 2.
E46 A compound of embodiment E32, or a pharmaceutically acceptable salt thereof, wherein the compound is selected from the group consisting of:

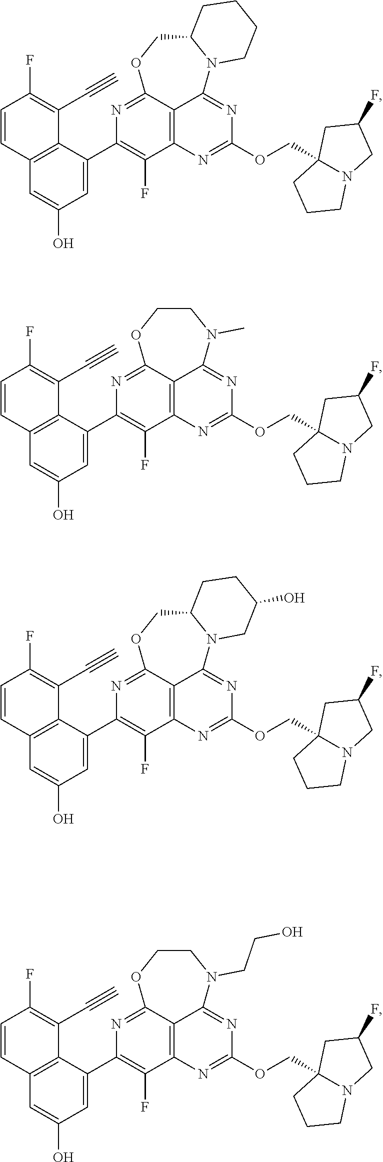 [SMILES: C#Cc1c(F)ccc2cc(O)cc(-c3nc4c5c(nc(OC[C@@]67CCCN6C[C@H](F)C7)nc5c3F)N3CCCC[C@H]3CO4)c12 ; C#Cc1c(F)ccc2cc(O)cc(-c3nc4c5c(nc(OC[C@@]67CCCN6C[C@H](F)C7)nc5c3F)N(C)CCO4)c12 ; C#Cc1c(F)ccc2cc(O)cc(-c3nc4c5c(nc(OC[C@@]67CCCN6C[C@H](F)C7)nc5c3F)N3C[C@@H](O)CC[C@H]3CO4)c12 ; C#Cc1c(F)ccc2cc(O)cc(-c3nc4c5c(nc(OC[C@@]67CCCN6C[C@H](F)C7)nc5c3F)N(CCO)CCO4)c12]

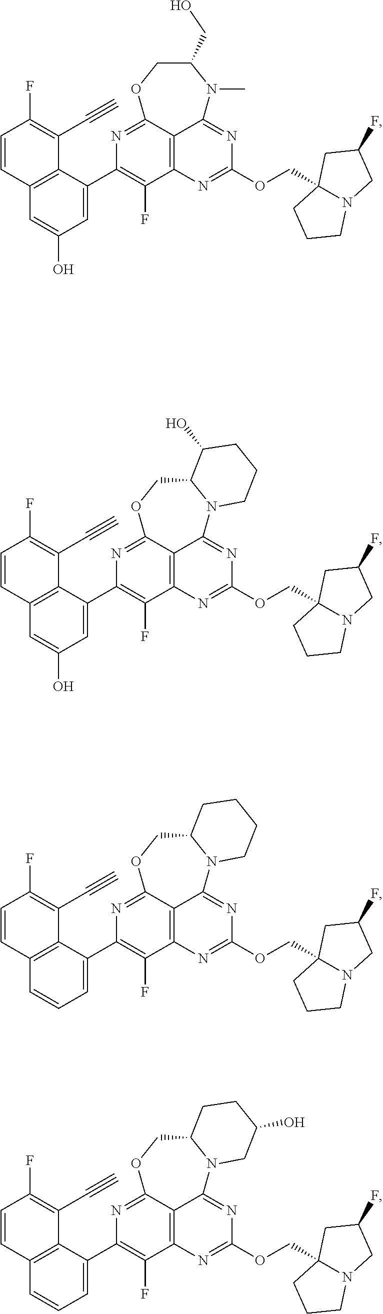 [SMILES: C#Cc1c(F)ccc2cc(O)cc(-c3nc4c5c(nc(OC[C@@]67CCCN6C[C@H](F)C7)nc5c3F)N(C)[C@@H](CO)CO4)c12 ; C#Cc1c(F)ccc2cc(O)cc(-c3nc4c5c(nc(OC[C@@]67CCCN6C[C@H](F)C7)nc5c3F)N3CCC[C@@H](O)[C@H]3CO4)c12 ; C#Cc1c(F)ccc2cccc(-c3nc4c5c(nc(OC[C@@]67CCCN6C[C@H](F)C7)nc5c3F)N3CCCC[C@H]3CO4)c12 ; C#Cc1c(F)ccc2cccc(-c3nc4c5c(nc(OC[C@@]67CCCN6C[C@H](F)C7)nc5c3F)N3C[C@@H](O)CC[C@H]3CO4)c12]

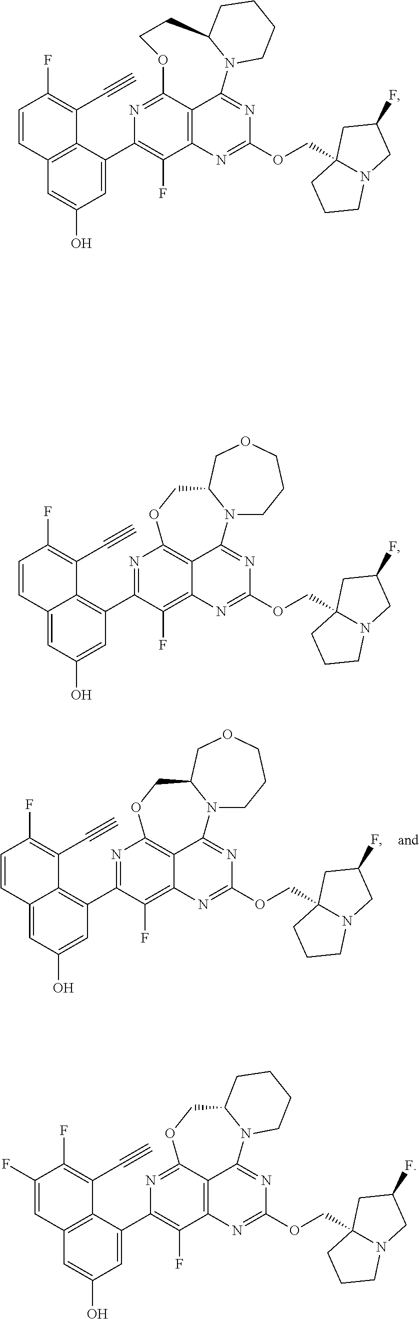

E47 A compound that is:

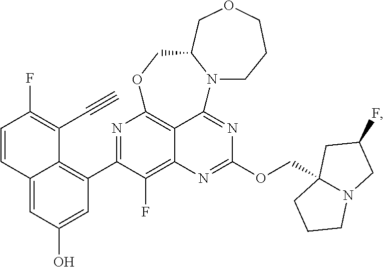 [SMILES: C#Cc1c(F)ccc2cc(O)cc(-c3nc4c5c(nc(OC[C@@]67CCCN6C[C@H](F)C7)nc5c3F)N3CCCOC[C@H]3CO4)c12]

or a pharmaceutically acceptable salt thereof.
E48 A compound that is:

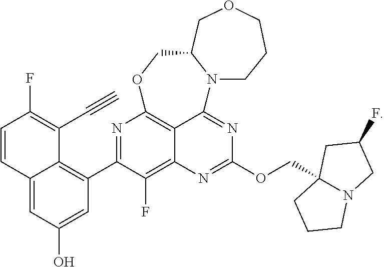 [SMILES: C#Cc1c(F)ccc2cc(O)cc(-c3nc4c5c(nc(OC[C@@]67CCCN6C[C@H](F)C7)nc5c3F)N3CCCOC[C@H]3CO4)c12]

E49 A pharmaceutically acceptable salt of a compound, wherein the compound is:

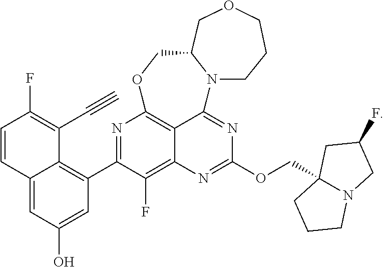 [SMILES: C#Cc1c(F)ccc2cc(O)cc(-c3nc4c5c(nc(OC[C@@]67CCCN6C[C@H](F)C7)nc5c3F)N3CCCOC[C@H]3CO4)c12]

E50 A pharmaceutical composition comprising a compound of any one of embodiments E32 to E47, or a pharmaceutically acceptable salt thereof, and at least one pharmaceutically acceptable excipient.
E51 A method for treating cancer, comprising administering to a subject in need thereof a therapeutically effective amount of a compound of any one of embodiments E32 to E47, or a pharmaceutically acceptable salt thereof.
E52 A method for treating cancer, comprising administering to a subject in need thereof a therapeutically effective amount of a compound of any one of embodiments E32 to E47, or a pharmaceutically acceptable salt thereof, as a single agent.
E53 A method for treating cancer, comprising administering to a subject in need thereof a therapeutically effective amount of a compound of any one of embodiments E32 to E47, or a pharmaceutically acceptable salt thereof, and further comprising administering a therapeutically effective amount of an additional anticancer therapeutic agent.
E54 A method for treating cancer of any one of embodiments E51 to E53, wherein the cancer is small cell lung cancer (NSCLC), pancreatic cancer, or colorectal cancer.
E55 A compound of any one of embodiments E32 to E47, or a pharmaceutically acceptable salt thereof, for use as a medicament.
E56 A compound of any one of embodiments E32 to E47, or a pharmaceutically acceptable salt thereof, for use in the treatment of cancer.
E57 A compound for use in the treatment of cancer according to embodiment E56, wherein said cancer is small cell lung cancer (NSCLC), pancreatic cancer, or colorectal cancer.
E58 Use of a compound of any one of embodiments E32 to E47, or a pharmaceutically acceptable salt thereof, for the manufacture of a medicament for the treatment of cancer.
E59 Use of a compound, or a pharmaceutically acceptable salt thereof, for the manufacture of a medicament for the treatment of cancer according to embodiment E58, wherein the cancer is small cell lung cancer (NSCLC), pancreatic cancer, or colorectal cancer.
E60 A method for the treatment of a disorder mediated by inhibition of KRAS G12C, KRAS G12D, and KRAS G12V receptors in a subject, comprising administering to the subject in need thereof a compound of any one of embodiments E32 to E47, or a pharmaceutically acceptable salt thereof, in an amount that is effective for treating the disorder.
E61 A pharmaceutical combination comprising a compound of any one of embodiments E32 to E47, or a pharmaceutically acceptable salt thereof, and at least one additional therapeutic agent or a pharmaceutically acceptable salt thereof, wherein said pharmaceutical combination is a fixed or non-fixed combination.
E62 A pharmaceutical composition comprising the pharmaceutical combination of embodiment E61 and at least one excipient.
E63 A compound of Formula (V):

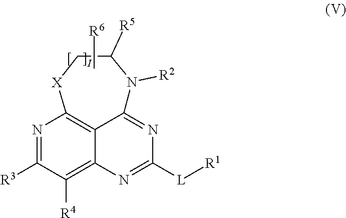

or a pharmaceutically acceptable salt thereof, wherein:
R¹ is selected from the group consisting of:

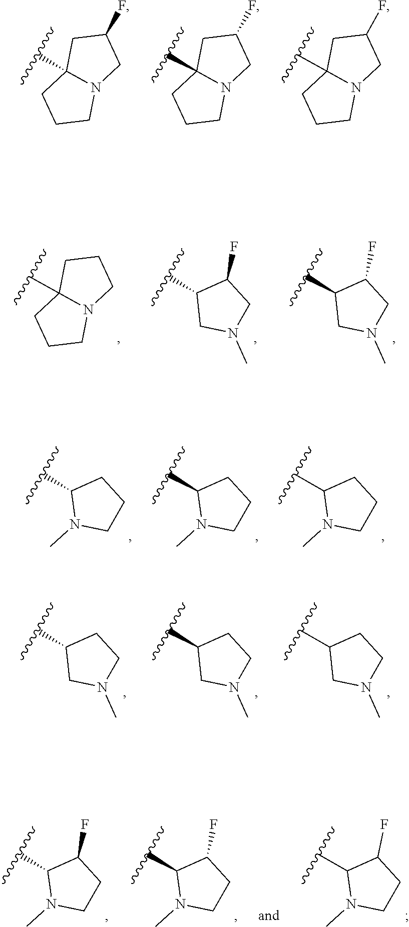

R² is C₁ alkyl, C₃ alkyl, —(C₁ alkylene)-OH, or —(C₃ alkylene)-OH;
R³ is selected from the group consisting of:

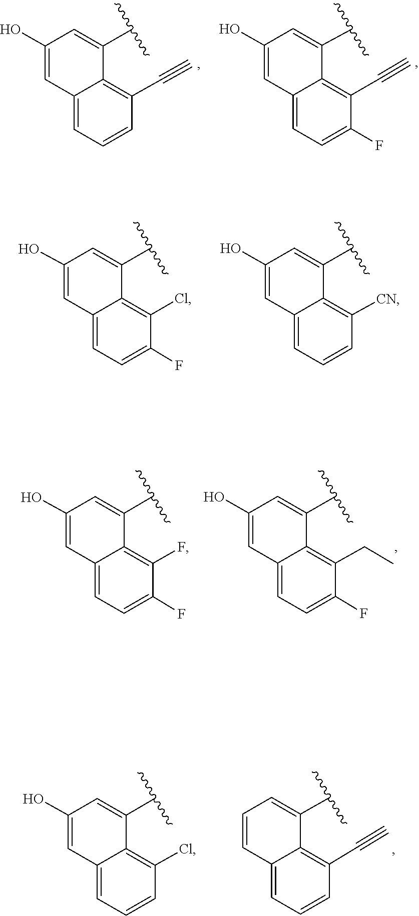

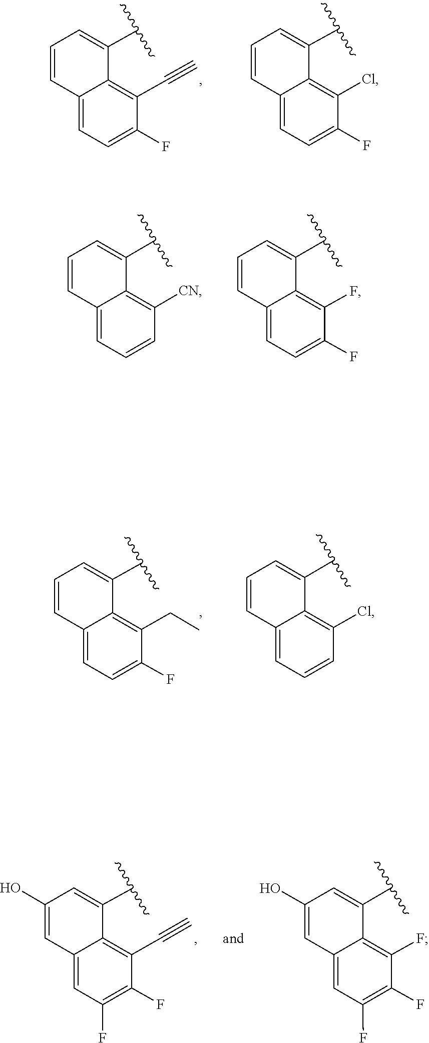

R⁵ is —(C₁ alkylene)-OH, or C₁ alkyl, wherein R² and R⁵ are optionally taken together to form a 7-membered heterocycloalkyl comprising one heteroatom O;
R⁶ represents one or two substituents selected from the group consisting of H, —OH, halogen, —(C₁-C₆ alkylene)-OH, —CN, —(C₁-C₆ alkylene)-CN, C₁-C₆ alkyl, C₃-C₆ cycloalkyl, C₁-C₆ fluoroalkyl, C₃-C₆ fluorocycloalkyl, and C₁-C₆ alkoxy;
L is a linker comprising one, two or three members independently selected from the group consisting of —O—, —S—, —NR⁷—, and —CR⁸R⁹—;
R⁷, R⁸, and R⁹ are each independently H or C₁-C₃ alkyl;
X is O, N, or S; and
l is 1 or 2.
E64 The compound of embodiment E63, or a pharmaceutically acceptable salt thereof, wherein R¹ is selected from the group consisting of:

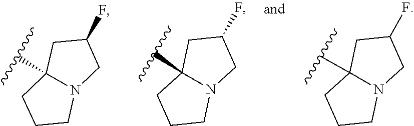

E65 The compound of embodiment E64, or a pharmaceutically acceptable salt thereof, wherein R¹ is:

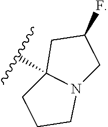 [SMILES: *[C@@]12CCCN1C[C@H](F)C2]

E66 The compound of any one of embodiments E63 to E 65, or a pharmaceutically acceptable salt thereof, wherein R² is C₃ alkyl, and R⁵ is —(C₁ alkylene)-OH.
E67 The compound of any one of embodiments E63 to E 65, or a pharmaceutically acceptable salt thereof, wherein R² is —(C₃ alkylene)-OH, and R⁵ is C₁ alkyl.
E68 The compound of embodiment E66, or a pharmaceutically acceptable salt thereof, wherein R² and R⁵ are taken together to form a 7-membered heterocycloalkyl comprising one heteroatom O.
E69 The compound of embodiment E67, or a pharmaceutically acceptable salt thereof, wherein R² and R⁵ are taken together to form a 7-membered heterocycloalkyl comprising one heteroatom O.
E70 The compound of any one of embodiments E63 to E 69, or a pharmaceutically acceptable salt thereof, wherein R³ is:

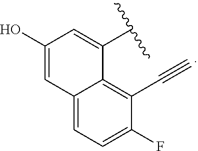 [SMILES: *c1cc(O)cc2ccc(F)c(C#C)c12]

E71 The compound of any one of embodiments E63 to E70, or a pharmaceutically acceptable salt thereof, wherein R⁴ is F.
E72 The compound of any one of embodiments E63 to E 71, or a pharmaceutically acceptable salt thereof, wherein X is O, l is 1.
E73 The compound of embodiment E63, or a pharmaceutically acceptable salt thereof, wherein the compound has Formula (VI):

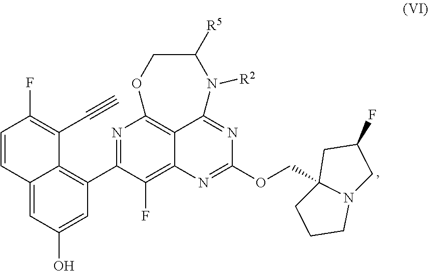

E74 The compound of embodiment E73, or a pharmaceutically acceptable salt thereof, wherein R² is C₃ alkyl, and R⁵ is —(C₁ alkylene)-OH.
E75 The compound of embodiment E73, or a pharmaceutically acceptable salt thereof, wherein R² is —(C₃ alkylene)-OH, and R⁵ is C₁ alkyl.
E76 The compound of embodiment E74, or a pharmaceutically acceptable salt thereof, wherein R² and R⁵ are taken together to form a 7-membered heterocycloalkyl comprising one heteroatom O.
E77 The compound of embodiment E75, or a pharmaceutically acceptable salt thereof, wherein R² and R⁵ are taken together to form a 7-membered heterocycloalkyl comprising one heteroatom O.
E78 A compound of Formula (VII)

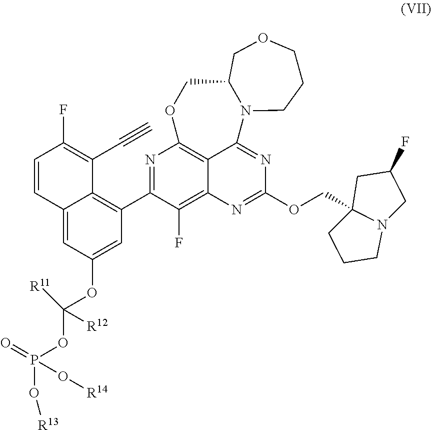

or a pharmaceutically acceptable salt thereof, wherein R¹¹, R¹², R¹³, and R¹⁴ are each independently H or C₁-C₃ alkyl.
E79 The compound of embodiment E78, or a pharmaceutically acceptable salt thereof, wherein R¹¹, R¹², R¹³, and R¹⁴ are each independently H or methyl.
E80 The compound of embodiment E78 or E79, or a pharmaceutically acceptable salt thereof, wherein the compound is

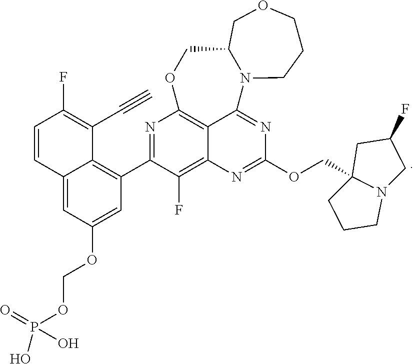 [SMILES: C#Cc1c(F)ccc2cc(OCOP(=O)(O)O)cc(-c3nc4c5c(nc(OC[C@@]67CCCN6C[C@H](F)C7)nc5c3F)N3CCCOC[C@H]3CO4)c12]

E81 A compound or a pharmaceutically acceptable salt thereof, selected from the group consisting of:

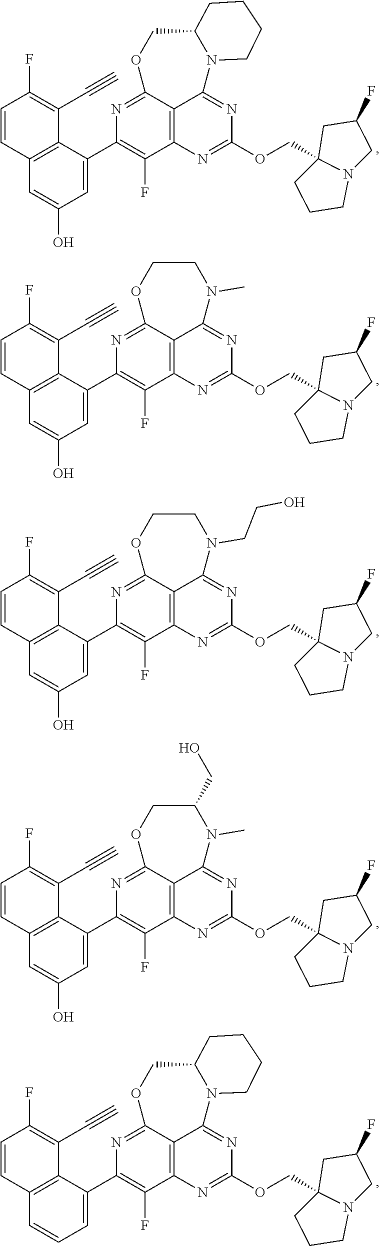 [SMILES: C#Cc1c(F)ccc2cc(O)cc(-c3nc4c5c(nc(OC[C@@]67CCCN6C[C@H](F)C7)nc5c3F)N3CCCC[C@H]3CO4)c12 ; C#Cc1c(F)ccc2cc(O)cc(-c3nc4c5c(nc(OC[C@@]67CCCN6C[C@H](F)C7)nc5c3F)N(C)CCO4)c12 ; C#Cc1c(F)ccc2cc(O)cc(-c3nc4c5c(nc(OC[C@@]67CCCN6C[C@H](F)C7)nc5c3F)N(CCO)CCO4)c12 ; C#Cc1c(F)ccc2cc(O)cc(-c3nc4c5c(nc(OC[C@@]67CCCN6C[C@H](F)C7)nc5c3F)N(C)[C@@H](CO)CO4)c12 ; C#Cc1c(F)ccc2cccc(-c3nc4c5c(nc(OC[C@@]67CCCN6C[C@H](F)C7)nc5c3F)N3CCCC[C@H]3CO4)c12]

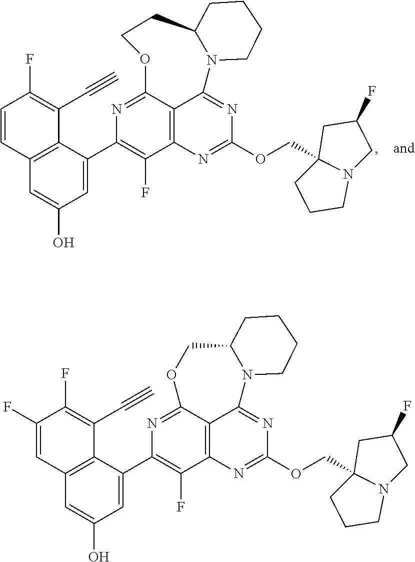

E82 A pharmaceutical composition comprising a compound of any one of embodiments E63 to E81, or a pharmaceutically acceptable salt thereof, and at least one pharmaceutically acceptable excipient.
E83 A method for treating cancer, comprising administering to a subject in need thereof a therapeutically effective amount of a compound of any one of embodiments E63 to E81, or a pharmaceutically acceptable salt thereof.
E84 A method for treating cancer, comprising administering to a subject in need thereof a therapeutically effective amount of a compound of any one of embodiments E63 to E81, or a pharmaceutically acceptable salt thereof, as a single agent.
E85 A method for treating cancer, comprising administering to a subject in need thereof a therapeutically effective amount of a compound of any one of embodiments E63 to E81, or a pharmaceutically acceptable salt thereof, and further comprising administering a therapeutically effective amount of an additional anticancer therapeutic agent.
E86 A method for treating cancer of any one of embodiments E83 to E85, wherein the cancer is small cell lung cancer (NSCLC), pancreatic cancer, or colorectal cancer.
E87 A compound of any one of embodiments E63 to E81, or a pharmaceutically acceptable salt thereof, for use as a medicament.
E88 A compound of any one of embodiments E63 to E81, or a pharmaceutically acceptable salt thereof, for use in the treatment of cancer.
E89 A compound for use in the treatment of cancer according to embodiment E88, wherein said cancer is small cell lung cancer (NSCLC), pancreatic cancer, or colorectal cancer.
E90 Use of a compound of any one of embodiments E63 to E81, or a pharmaceutically acceptable salt thereof, for the manufacture of a medicament for the treatment of cancer.
E91 Use of a compound, or a pharmaceutically acceptable salt thereof, for the manufacture of a medicament for the treatment of cancer according to embodiment E90, wherein the cancer is small cell lung cancer (NSCLC), pancreatic cancer, or colorectal cancer.
E92 A method for the treatment of a disorder mediated by inhibition of KRAS G12C, KRAS G12D, and KRAS G12V receptors in a subject, comprising administering to the subject in need thereof a compound of any one of embodiments E63 to E81, or a pharmaceutically acceptable salt thereof, in an amount that is effective for treating the disorder.
E93 A pharmaceutical combination comprising a compound of any one of embodiments E63 to E81, or a pharmaceutically acceptable salt thereof, and at least one additional therapeutic agent or a pharmaceutically acceptable salt thereof, wherein said pharmaceutical combination is a fixed or non-fixed combination.
E94 A pharmaceutical composition comprising the pharmaceutical combination of embodiment E93 and at least one excipient.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or a pharmaceutically acceptable salt thereof.

Furthermore, each of the embodiments described herein envisions within its scope pharmaceutically acceptable salts of the compounds, stereoisomers of the compounds, and pharmaceutically acceptable salts of the stereoisomers described herein.

### Definitions

Unless otherwise defined herein, scientific, and technical terms used in connection with the present invention have the meanings that are commonly understood by those of ordinary skill in the art.

The invention described herein suitably may be practiced in the absence of any element(s) not specifically disclosed herein.

“Compounds of the invention” include compounds of Formula (I) and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, and isotopically labelled versions thereof, where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of 5 mg) means that the parameter may vary by as much as 10% below or above the stated numerical value for that parameter. For example, a dose of about 5 mg means 5%±10%, i.e., it may vary between 4.5 mg and 5.5 mg.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” refers to fluoro, chloro, bromo and iodo (F, Cl, Br, I).

“Cyano” refers to a substituent having a carbon atom joined to a nitrogen atom by a triple bond, i.e., —C≡N (also depicted herein as “—CN”).

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 6 carbon atoms (“C₁-C₆ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, and the like.

“Fluoroalkyl” refers to an alkyl group, as defined herein, wherein from one to all of the hydrogen atoms of the alkyl group are replaced by fluoro atoms. Examples include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Alkylene” refers to a bivalent aliphatic hydrocarbon radical that has a specified number of carbon atoms. Alkylene groups may contain, but are not limited to, 1 to 6 carbon atoms (“C₁-C₆ alkylene”), or 1 to 2 carbon atoms (“C₁-C₂ alkylene”). Examples include —(CH₂)-(methylene) and —(CH₂—CH₂)— (ethylene).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 6 carbon atoms (“C₁-C₆ alkoxy”), or 1 to 3 carbon atoms (“C₁-C₃ alkoxy”). Alkoxy groups include, but are not limited to, methoxy, ethoxy, n-propoxy, and the like.

“Alkynyl” refers to an alkyl group, as defined herein, consisting of at least two carbon atoms and at least one carbon-carbon triple bond. Alkynyl may contain 2-3 carbon atoms (“C₂-C₃ alkynyl”). Examples include, but are not limited to, ethynyl, 1-propynyl, 2-propynyl, and the like.

“Cycloalkyl” refers to a fully saturated hydrocarbon ring system that has the specified number of carbon atoms, which may be a monocyclic, bridged or fused bicyclic or polycyclic ring system that is connected to the base molecule through a carbon atom of the cycloalkyl ring. Cycloalkyl groups may contain, but are not limited to, 3 to 10 carbon atoms (“C₃-C₁₀cycloalkyl”), 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein.

“Fluorocycloalkyl” refers to a cycloalkyl group, as defined herein, wherein from one to all of the hydrogen atoms of the alkyl group are replaced by fluoro atoms. Examples include, but are not limited to, fluorocyclcopropyl, fluorocyclcobutyl, fluorocyclcopentyl and fluorocyclcohexyl,

“Heterocycloalkyl” refers to a fully saturated ring system containing the specified number of ring atoms and containing at least one heteroatom selected from N, O and S as a ring member, where ring S atoms are optionally substituted by one or two oxo groups (i.e., S(O)_{q}, where q is 0, 1 or 2) and where the heterocycloalkyl ring is connected to the base molecule via a ring atom, which may be C or N. Heterocycloalkyl rings include monocyclic or polycyclic such as bicyclic rings. Heterocycloalkyl rings also include rings which are spirocyclic, bridged, or fused to one or more other heterocycloalkyl or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(O)_{q} as ring members, or 1 to 3 ring heteroatoms, or 1 to 2 ring heteroatoms, provided that such heterocycloalkyl rings do not contain two contiguous oxygen or sulfur atoms.

Heterocycloalkyl rings may be optionally substituted, unsubstituted or substituted, as further defined herein. Such substituents may be present on the heterocyclic ring attached to the base molecule, or on a monocyclic, bicyclic, tricyclic, spirocyclic, bridged or fused ring attached thereto.

Heterocycloalkyl rings may include, but are not limited to, 4-12 membered heterocyclyl groups, for example 5-8 or 4-6 membered heterocycloalkyl groups, in accordance with the definition herein. Examples of heterocycloalkyl ring group of the present invention may include, but are not limited to aziridinyl, azetidinyl, pyrrolidinyl, piperidinyl, piperazinyl, morpholinyl, thiomorpholinyl, azepanyl, oxaazepanyl, thieazepanyl, a radical of hexahydro-1H-pyrrolizine ring, a radical of 8-oxa-3-azabicyclo[3.2.1]octane ring, a radical of 3-azabicyclo[3.2.1]octane ring, a radical of 6-azabicyclo[3.2.1]octane ring, or a radical of 3-azabicyclo[3.2.0]heptane ring.

“Aryl” or “aromatic” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 10 carbon atoms (“C₈-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, quinolinyl, isoquinolinyl, purinyl, triazinyl, naphthyridinyl, cinnolinyl, quinazolinyl, quinoxalinyl, carbazolyl, imidazo[4,5-b]pyridinyl, imidazo[4,5-c]pyridinyl, pyrazolo[4,3-d]pyidinyl, pyrazolo[4,3-c]pyidinyl, pyrazolo[3,4-c]pyidinyl, pyrazolo[3,4-b]pyidinyl, isoindolyl, purinyl, indolininyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, azaquinazolinyl, phthalazinyl, (pyrido[3,2-d]pyrimidinyl, (pyrido[4,3-d]pyrimidinyl, (pyrido[3,4-d]pyrimidinyl, (pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

“Amino” refers to a group —NH₂, which is unsubstituted. Where the amino is described as substituted or optionally substituted, the term includes groups of the form —NRxRy, where each of Rx and Ry is defined as further described herein. For example, “alkylamino” refers to a group —NRxRy, wherein one of Rx and Ry is an alkyl moiety and the other is H, and “dialkylamino” refers to —NRxRy wherein both of Rx and Ry are alkyl moieties, where the alkyl moieties have the specified number of carbon atoms (e.g., —NH(C₁-C₄ alkyl) or —N(C₁-C₄ alkyl)₂).

A wavy line “ [symbol] ” used in a chemical structure in the present disclosure refers to the [symbol] point of the attachment of a substituent.

The term “pharmaceutically acceptable” means the substance (e.g., the compounds described herein) and any salt thereof, or composition containing the substance or salt of the invention is suitable for administration to a subject or patient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds of Formula (I) may also include other salts of such compounds which are not necessarily pharmaceutically acceptable salts, which may be useful as intermediates for one or more of the following: 1) preparing compounds of Formula (I); 2) purifying compounds of Formula (I); 3) separating enantiomers of compounds of Formula (I); or 4) separating diastereomers of compounds of Formula (I).

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt thereof, and one or more pharmaceutically acceptable solvent molecules, for example, ethanol. The term ‘hydrate’ is employed when said solvent is water.

In addition, the compounds of Formula (I) may also include other solvates of such compounds which are not necessarily pharmaceutically acceptable solvates, which may be useful as intermediates for one or more of the following: 1) preparing compounds of Formula (I); 2) purifying compounds of Formula (I); 3) separating enantiomers of compounds of Formula (I); or 4) separating diastereomers of compounds of Formula (I).

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid Form

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃—Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers.

The pharmaceutically acceptable salts of compounds of the invention may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H and ³H, carbon, such as ¹¹C ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example those incorporating a radioactive isotope, are useful in one or both of drug or substrate tissue distribution studies. The radioactive isotopes tritium, i.e., ³H, and carbon-14, i.e., ¹⁴C, are particularly useful for this purpose in view of their ease of incorporation and ready means of detection.

Substitution with deuterium, i.e., ²H, may afford certain therapeutic advantages resulting from greater metabolic stability.

Substitution with positron emitting isotopes, such as ¹¹C, ¹⁸F, ¹⁵O and ¹³N, may be useful in Positron Emission Topography (PET) studies for examining substrate receptor occupancy.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, the deuterium compound is selected from any one of the compounds set forth in Table 2 shown in the Examples section.

In some embodiments, one or more hydrogen atoms on certain metabolic sites on the compounds of the invention are deuterated.

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

### Prodrugs

A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or (e) an oxime or imine derivative of a carbonyl group when present in a compound of the invention.

Some specific examples of prodrugs in accordance with the invention include:
(i) when a compound of the invention contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., ^{t}BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the invention contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.
(vii) when a compound of the invention contains an alcohol functionality (—OH), replacement of the hydrogen of the alcohol functionality with a group selected the set below:

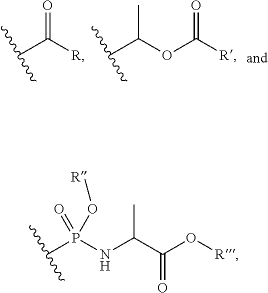

wherein R, R′, R″, R′″ are (C₁-C₈)alkyl or (C₁-C₈)alkoxy and can be linear, branched or cyclic. Some preferred prodrugs can be prepared through —OH on a C₆-C₁₀ bicyclic aryl or a 4-12 membered bicyclic heteroaryl. Some more preferred prodrugs can be prepared through —OH on a naphthyl.

Certain compounds of the invention may themselves act as prodrugs of other compounds the invention It is also possible for two compounds of the invention to be joined together in the form of a prodrug. In certain circumstances, a prodrug of a compound of the invention may be created by internally linking two functional groups in a compound of the invention, for instance by forming a lactone.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to:
(i) where the compound of the invention contains an alkyl group, a hydroxyalkyl derivative thereof (—CH>—COH):
(ii) where the compound of the invention contains an alkoxy group, a hydroxy derivative thereof (—OR->—OH);
(iii) where the compound of the invention contains a tertiary amino group, a secondary amino derivative thereof (—NRR->—NHR or —NHR);
(iv) where the compound of the invention contains a secondary amino group, a primary derivative thereof (—NHR->—NH₂);
(v) where the compound of the invention contains a phenyl moiety, a phenol derivative thereof (-Ph->-PhOH);
(vi) where the compound of the invention contains an amide group, a carboxylic acid derivative thereof (—CONH₂->COOH); and
(vii) where the compound contains a hydroxy or carboxylic acid group, the compound may be metabolized by conjugation, for example with glucuronic acid to form a glucuronide.

Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the invention comprises pharmaceutical compositions. For pharmaceutical composition purposes, the compound per se or pharmaceutically acceptable salt thereof will simply be referred to as the compounds of the invention.

A “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

The term “excipient” is used herein to describe any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250, 500 or 1000 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

Liposome containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example, “Amorphous Solid Dispersions Theory and Practice” by Navnit Shah et al.

### Administration and Dosing

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

Typically, a compound of the invention is administered in an amount effective to treat a condition as described herein. The compounds of the invention may be administered as compound per se, or alternatively, as a pharmaceutically acceptable salt. For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired.

### Therapeutic Methods and Uses

The compounds of the invention may inhibit the activities of all KRAS G12C, KRAS G12D, and KRAS G12V receptors, and may be useful in the treatment, prevention, suppression, and amelioration of diseases such as cancers, disorders and conditions mediated by any of KRAS G12C, KRAS G12D, and KRAS G12V receptors, or a combination thereof.

Cancers to be treated include squamous cell carcinoma, basal cell carcinomas, myeloma, small-cell lung cancer, non-small cell lung cancer, glioma, Hodgkin's lymphoma, non-Hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, uterine cancer, bladder cancer, including non-muscular invasive bladder cancer, hepatoma, breast cancer, and head and neck cancer.

Preferably, the compounds of the present invention may be useful for the treatment of lung cancers such as non-small cell lung cancer (NSCLC), pancreatic cancer, colorectal cancer, breast cancer, blood cancers, gynecological cancers, prostate cancer, or skin cancer. See Mustachio, L., Targeting KRAS in Cancer: Promising Therapeutic Strategies, Cancers, 2021, 13, 1204.

More preferably, the compounds of the present invention may be useful for the treatment of non-small cell lung cancer (NSCLC), pancreatic cancer, and colorectal cancer.

### Co-Administration

The compounds of the invention may be used alone, or in combination with one or more other therapeutic agents. The invention provides any of the uses, methods or compositions as defined herein wherein the compound of the invention, or pharmaceutically acceptable salt thereof, is used in combination with one or more other therapeutic anticancer agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the invention and the one or more other therapeutic agents may be administered as a fixed or non-fixed combination of the active ingredients. The term “fixed combination” means a compound of the invention, or a pharmaceutically acceptable salt thereof, and the one or more therapeutic agents, are both administered to a subject simultaneously in a single composition or dosage. The term “non-fixed combination” means that a compound of the invention, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

Classes of additional chemotherapeutic agents, which can be administered in combination with a compound of this invention, include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists; IL-2 receptor agonist (recombinant cytokines or agonists for cytokine receptors); and anti-sense oligonucleotides or oligonucleotides derivatives that inhibit expression of genes implicated in abnormal cell proliferation or tumor growth.

Other additional chemotherapy agents include not only taxanes or platinum agents but also HER2 targeted agents, e.g., trastuzumab.

In another embodiment, such additional anti-cancer therapeutic agents include compounds derived from the following classes: mitotic inhibitors, alkylating agents, antimetabolites, antitumor antibiotics, anti-angiogenesis agents, topoisomerase I and II inhibitors, plant alkaloids, spindle poison plant alkaloids, MCT4 inhibitors; MAT2a inhibitors; alk/c-Met/ROS inhibitors (including crizotinib or lorlatinib); mTOR inhibitors (including temsirolimus or gedatolisib); src/abl inhibitors (including bosutinib); cyclin-dependent kinase (CDK) inhibitors (including palbociclib, PF-06873600); erb inhibitors (including dacomitinib); PARP inhibitors (including talazoparib); SMO inhibitors (including glasdegib); EGFR T790M inhibitors; PRMT5 inhibitors; TGFβR1 inhibitors; growth factor inhibitors; cell cycle inhibitors, biological response modifiers; enzyme inhibitors; and cytotoxics.

In another embodiment, such additional anti-cancer therapeutic agents include compounds derived from an anti-angiogenesis agent, including for example tyrosine kinase/vascular endothelial growth factor (VEGF) receptor (VEGFR) inhibitors (including sunitinib, axitinib, sorafenib, and tivozanib), TIE-2 inhibitors, PDGFR inhibitors, angiopoetin inhibitors, PKCP inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent™), bevacizumab (Avastin™) axitinib (Inlyta™), SU 14813 (Pfizer), and AG 13958 (Pfizer). Additional anti-angiogenesis agents include vatalanib (CGP 79787), pegaptanib octasodium (Macugen™), vandetanib (Zactima™), PF-0337210 (Pfizer), SU 14843 (Pfizer), AZD 2171 (AstraZeneca), ranibizumab (Lucentis™), Neovastat™ (AE 941), tetrathiomolybdata (Coprexa™), AMG 706 (Amgen), VEGF Trap (AVE 0005), CEP 7055 (Sanofi-Aventis), XL 880 (Exelixis), telatinib (BAY 57-9352), and CP-868,596 (Pfizer). Other anti-angiogenesis agents include enzastaurin (LY 317615), midostaurin (CGP 41251), perifosine (KRX 0401), teprenone (Selbex™) and UCN 01 (Kyowa Hakko). Other examples of anti-angiogenesis agents include celecoxib (Celebrex™), parecoxib (Dynastat™), deracoxib (SC 59046), lumiracoxib (Preige™), valdecoxib (Bextra™), rofecoxib (Vioxx™), iguratimod (Careram™), IP 751 (Invedus), SC-58125 (Pharmacia) and etoricoxib (Arcoxia™). Yet further anti-angiogenesis agents include exisulind (Aptosyn™), salsalate (Amigesic™), diflunisal (Dolobid™), ibuprofen (Motrin™), ketoprofen (Orudis™), nabumetone (Relafen™), piroxicam (Feldene™), naproxen (Aleve™, Naprosyn™), diclofenac (Voltaren™) indomethacin (Indocin™), sulindac (Clinoril™), tolmetin (Tolectin™), etodolac (Lodine™) ketorolac (Toradol™), and oxaprozin (Daypro™). Yet further anti-angiogenesis agents include ABT 510 (Abbott), apratastat (TMI 005), AZD 8955 (AstraZeneca), incyclinide (Metastat™), and PCK 3145 (Procyon). Yet further anti-angiogenesis agents include acitretin (Neotigason™) plitidepsin (Aplidine™), cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™).

In another embodiment, such additional anti-cancer therapeutic agents include compounds derived from hormonal agents and antagonists. Examples include where anti-hormonal agents act to regulate or inhibit hormone action on tumors such as anti-estrogens and selective estrogen receptor modulators (SERMs), and a selective estrogen receptor degrader (SERD) including tamoxifen, raloxifene, droloxifene, 4-hydroxytamoxifen, trioxifene, keoxifene, LY117018, onapristone, toremifene (Fareston), and fulvestrant. Examples also include aromatase inhibitors that inhibit the enzyme aromatase, which regulates estrogen production in the adrenal glands, and include compounds like 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, fluridil, apalutamide, enzalutamide, cimetidine and goserelin.

In another embodiment, such additional anti-cancer therapeutic agents include compounds derived from signal transduction inhibitors, such as inhibitors of protein tyrosine kinases and/or serine/threonine kinases: a signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1R inhibitors, MEK (including binimetinib (Mektovi™)), c-Kit inhibitors, FLT-3 inhibitors, K-Ras inhibitors, PI3 kinase inhibitors, JAK inhibitors, STAT inhibitors, Raf kinase inhibitors, BRAF (including encorafenib (Braftovi™)), Akt inhibitors, mTOR inhibitor, P70S6 kinase inhibitors, inhibitors of the WNT pathway and multi-targeted kinase inhibitors.

In another embodiment, such additional anti-cancer therapeutic agents include docetaxel, paclitaxel, paclitaxel protein-bound particles, cisplatin, carboplatin, oxaliplatin, capecitabine, gemcitabine or vinorelbine.

In another embodiment, such additional anti-cancer therapeutic agents include compounds derived from an epigenetic modulator, where examples include an inhibitor of EZH2 (including PF-06821497), SMARCA4, PBRM1, ARID1A, ARID2, ARID1B, DNMT3A, TET2, MLL1/2/3, NSD1/2, SETD2, BRD4, DOT1L, HKMTsanti, PRMT1-9, LSD1, UTX, IDH1/2 or BCL6.

In another embodiment, such additional anti-cancer therapeutic agents include compounds that are immuno-oncology agents, including immunomodulatory agents.

In another embodiment, combinations with pattern recognition receptors (PRRs) are contemplated. PRRs are receptors that are expressed by cells of the immune system and that recognize a variety of molecules associated with pathogens and/or cell damage or death. PRRs are involved in both the innate immune response and the adaptive immune response. PRR agonists may be used to stimulate the immune response in a subject. There are multiple classes of PRR molecules, including toll-like receptors (TLRs), RIG-1-like receptors (RLRs), nucleotide-binding oligomerization domain (NOD)-like receptors (NLRs), C-type lectin receptors (CLRs), and Stimulator of Interferon Genes (STING) protein.

The STING protein functions as both a cytosolic DNA sensor and an adaptor protein in Type 1 interferon signaling. The terms “STING” and “stimulator of interferon genes” refer to any form of the STING protein, as well as variants, isoforms, and species homologs that retain at least a part of the activity of STING. Unless indicated differently, such as by specific reference to human STING, STING includes all mammalian species of native sequence STING, e.g. human, monkey, and mouse STING is also known as—TMEM173.

“STING agonist” as used herein means, any molecule, which upon binding to STING, (1) stimulates or activates STING, (2) enhances, increases, promotes, induces, or prolongs an activity, function, or presence of STING, or (3) enhances, increases, promotes, or induces the expression of STING. STING agonists useful in the any of the treatment method, medicaments and uses of the present invention include, for example, nucleic acid ligands which bind STING.

Examples of STING agonists that are useful in the treatment methods, medicaments, and uses of the present invention include various immunostimulatory nucleic acids, such as synthetic double stranded DNA, cyclic di-GMP, cyclic-GMP-AMP (cGAMP), synthetic cyclic dinucleotides (CDN) such as MK-1454 and ADU-S100 (MIW815), and small molecules such as WO2019027858, WO20180093964, WO2017175156, WO2017175147.

Therapeutic antibodies may have specificity against a variety of different antigens. For example, therapeutic antibodies may be directed to a tumor associated-antigen, such that binding of the antibody to the antigen promotes death of the cell expressing the antigen. In other example, therapeutic antibodies may be directed to an antigen on an immune cell, such that binding of the antibody prevents downregulation of the activity of the cell expressing the antigen (and thereby promotes activity of the cell expressing the antigen). In some situations, a therapeutic antibody may function through multiple different mechanisms (for example, it may both i) promote death of the cell expressing the antigen, and ii) prevent the antigen from causing down-regulation of the activity of immune cells in contact with the cell expressing the antigen).

In another embodiment, such additional anti-cancer therapeutic agents include antibodies that would be blocking or inhibitory at the target: CTLA-4 (including ipilimumab or tremelimumab), PD-1 or PD-L1 (including atezolizumab, avelumab, cemiplimab, durvalumab, nivolumab, sasanlimab, or pembrolizumab), LAG-3, TIM-3, or TIGIT.

In another embodiment, such additional anti-cancer therapeutic agents include antibodies that are agonists of 4-1 BB, OX40, GITR, ICOS, or CD40.

In another embodiment the anti-cancer therapy may be a CAR-T-cell therapy.

Examples of a therapeutic antibody include: an anti-OX40 antibody, an anti-4-1 BB antibody, an anti-HER2 antibody (including an anti-HER2 antibody-drug conjugate (ADC)), a bispecific anti-CD47/anti-PD-L1 antibody, and a bispecific anti-P-cadherin/anti-CD3 antibody. Examples of cytotoxic agents that may be incorporated in an ADC include an anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, a pladienolide, and stereoisomers, isosteres, analogs, or derivatives thereof. Exemplary immunomodulating agents that may be incorporated in an ADC include gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, cytokines, stem cell growth factors, lymphotoxins, tumor necrosis factor (TNF), hematopoietic factors, interleukins (e.g., interleukin-1 (IL-1), IL-2, IL-3, IL-6, IL-10, IL-12, IL-15, IL-18, and IL-21), colony stimulating factors (e.g., granulocyte-colony stimulating factor (G-CSF) and granulocyte macrophage-colony stimulating factor (GM-CSF)), interferons (e.g., interferons-.alpha., -.beta. and -.gamma), the stem cell growth factor designated “S 1 factor,” erythropoietin and thrombopoietin, or a combination thereof.

Additional examples of therapeutic antibodies may include the following antigens where exemplary antibodies directed to the antigen are also included below (in brackets/parenthesis after the antigen). The antigens as follow may also be referred to as “target antigens” or the like herein. Target antigens for therapeutic antibodies herein include, for example: 4-1 BB (e.g. utomilumab); 5T4; A33; alpha-folate receptor 1 (e.g. mirvetuximab soravtansine); Alk-1; BCMA [e.g. see U.S. Pat. No. 9,969,809]; BTN1A1 (e.g. see WO2018222689); CA-125 (e.g. abagovomab); Carboanhydrase IX; CCR2; CCR4 (e.g. mogamulizumab); CCR5 (e.g. leronlimab); CCR8; CD3 [e.g. blinatumomab (CD3/CD19 bispecific), CD3/P-cadherin bispecific, CD3/BCMA bispecific] CD19 (e.g. blinatumomab, MOR208); CD20 (e.g. ibritumomab tiuxetan, obinutuzumab, ofatumumab, rituximab, ublituximab); CD22 (inotuzumab ozogamicin, moxetumomab pasudotox); CD25; CD28; CD30 (e.g. brentuximab vedotin); CD33 (e.g. gemtuzumab ozogamicin); CD38 (e.g. daratumumab, isatuximab), CD40; CD-40L; CD44v6; CD47 (e.g. Hu5F9-G4, CC-90002, SRF231, B6H12); CD52 (e.g. alemtuzumab); CD56; CD63; CD79 (e.g. polatuzumab vedotin); CD80; CD123; CD276/B7-H3 (e.g. omburtamab); CDH17; CEA; CIhCG; CTLA-4 (e.g. ipilimumab, tremelimumab), CXCR4; desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvIII; Endosialin; EpCAM (e.g. oportuzumab monatox); FAP; Fetal Acetylcholine Receptor; FLT3 (e.g. see WO2018/220584); GD2 (e.g. dinutuximab, 3F8); GD3; GITR; GloboH; GM1; GM2; HER2/neu [e.g. margetuximab, pertuzumab, trastuzumab; ado-trastuzumab emtansine, trastuzumab duocarmazine, [see U.S. Pat. No. 8,828,401]; HER3; HER4; ICOS; IL-10; ITG-AvB6; LAG-3 (e.g. relatlimab); Lewis-Y; LG; Ly-6; M-CSF [see U.S. Pat. No. 7,326,414]; MCSP; mesothelin; MUC1; MUC2; MUC3; MUC4; MUCSAC; MUC5B; MUC7; MUC16; Notch1; Notch3; Nectin-4 (e.g. enfortumab vedotin); OX40 [see U.S. Pat. No. 7,960,515]; P-Cadherein [see WO2016/001810]; PCDHB2; PDGFRA (e.g. olaratumab); Plasma Cell Antigen; PolySA; PSCA; PSMA; PTK7 [see U.S. Pat. No. 9,409,995]; Ror1; SAS; SCRx6; SLAMF7 (e.g. elotuzumab); SHH; SIRPa (e.g. ED9, Effi-DEM); STEAP; TGF-beta; TIGIT; TIM-3; TMPRSS3; TNF-alpha precursor; TROP-2 (e.g sacituzumab govitecan); TSPAN8; VEGF (e.g. bevacizumab, brolucizumab); VEGFR1 (e.g. ranibizumab); VEGFR2 (e.g. ramucirumab, ranibizumab); Wue-1.

Exemplary imaging agents that may be included in an ADC include fluorescein, rhodamine, lanthanide phosphors, and their derivatives thereof, or a radioisotope bound to a chelator. Examples of fluorophores include, but are not limited to, fluorescein isothiocyanate (FITC) (e.g., 5-FITC), fluorescein amidite (FAM) (e.g., 5-FAM), eosin, carboxyfluorescein, erythrosine, Alexa Fluor® (e.g., Alexa 350, 405, 430, 488, 500, 514, 532, 546, 555, 568, 594, 610, 633, 647, 660, 680, 700, or 750), carboxytetramethylrhodamine (TAMRA) (e.g., 5,-TAMRA), tetramethylrhodamine (TMR), and sulforhodamine (SR) (e.g., SR101). Examples of chelators include, but are not limited to, 1,4,7,10-tetraazacyclododecane-N,N′,N″,N′″-tetraacetic acid (DOTA), 1,4,7-triazacyclononane-1,4,7-triacetic acid (NOTA), 1,4,7-triazacyclononane, 1-glutaric acid-4,7-acetic acid (deferoxamine), diethylenetriaminepentaacetic acid (DTPA), and 1,2-bis(o-aminophenoxy)ethane-N,N,N′,N′-tetraacetic acid) (BAPTA).

Exemplary therapeutic proteins that may be included in an ADC include a toxin, a hormone, an enzyme, and a growth factor.

Exemplary biocompatible polymers that may be incorporated in an ADC include water-soluble polymers, such as polyethylene glycol (PEG) or its derivatives thereof and zwitterion-containing biocompatible polymers (e.g., a phosphorylcholine containing polymer).

Exemplary biocompatible polymers that may be incorporated in an ADC include anti-sense oligonucleotides.

The invention also concerns the use of radiation in combination with any anti-cancer therapeutic agent administered herein. More specifically, compounds of the invention can be administered in combination with additional therapies, such as radiation therapy and/or chemotherapy.

These agents and compounds of the invention may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage and a container for the dosage.

### Synthetic Methods

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains an amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-tert-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

### General Experimental Details

¹H and ¹⁹F Nuclear Magnetic Resonance (NMR) spectra were recorded on Bruker XWIN-NMR (400 or 700 MHz) spectrometer. ¹H and ¹⁹F resonances are reported in parts per million (ppm) downfield from tetramethylsilane. ¹H NMR data are reported as multiplicity (e.g. s, singlet; d, doublet; t, triplet; q, quartet; quint, quintuplet; dd, doublet of doublets; dt, doublet of triplets; br s, broad singlet). For spectra obtained in CDCl₃, DMSO-d₆, and CD₃OD, the residual protons (7.27, 2.50, and 3.31 ppm, respectively) were used as the internal reference. All observed coupling constants, J, are reported in Hertz (Hz). Exchangeable protons are not always observed.

Optical rotations were determined on a Jasco P-2000 or a Rudolph Autopol IV polarimeter. All final compounds were purified to ≥95% purity, unless otherwise specified. When absolute stereochemistry is known, (R,S) labels are used. When absolute stereochemistry is not known, the software-generated names are modified to include (+)- and (−)-prefixes according to the optical rotations, and (R*/S*) labels are used to show relative configuration.

Mass spectra, MS (m/z), were recorded using either electrospray ionization (ESI) or atmospheric pressure chemical ionization (APCI). Where relevant and unless otherwise stated, the m/z data provided are for isotopes ¹⁹F, 35Cl, ⁷⁹Br and ¹²⁷I.

The nomenclature is written as described by IUPAC (International Union of Pure and Applied Chemistry generated within Perkin Elmers Chemdraw 18.0.0.231. The naming convention provided with Perkin Elmers Chemdraw 18.0.0.231 is well known by those skilled in the art and it is believed that the naming convention provided with Perkin Elmers Chemdraw 18.0.0.231 generally comports with the IUPAC (International Union for Pure and Applied Chemistry) recommendations on Nomenclature of Organic Chemistry and the CAS Index rules.

### Abbreviations

aq is aqueous;
Bn is benzyl;
Boc is tert-butoxycarbonyl;
Boc₂O is di-tert-butyl dicarbonate;
br is broad;
tBu is tert-butyl;
° C. is degrees celcius;
CDCl₃ is deutero-chloroform;
δ is chemical shift;
d is doublet;
dd is doublet of doublets;
ddd is doublet of doublet of doublets;
dt is doublet of triplets;
DCM is dichloromethane; methylene chloride;
DIPEA is N-ethyldiisopropylamine, also known as N,N-diisopropylethylamine;
DMAP is 4-dimethylaminopyridine;
DMF is N,N-dimethylformamide;
DMSO is dimethyl sulfoxide;
DMSO-d₆ is deuterodimethylsulfoxide;
ee is enantiomeric excess;
ESI is electrospray ionization;
Et₂O is diethyl ether;
EtOAc is ethyl acetate;
EtOH is ethanol;
Et₃N is triethylamine;
g is gram;
HPLC is high pressure liquid chromatography;
hr(s) is hour(s);
L is liter;
LCMS is liquid chromatography mass spectrometry;
m is multiplet;
M is molar;
m-CPBA is 3-chloroperbenzoic acid;
MeOD_d₄ is deuterated methanol;
MeOH is methanol;
2-MeTHF is 2-methyl tetrahydrofuran;
mg is milligram;
MHz is mega Hertz;
min(s) is minute(s);
mL is milliliter;
mmol is millimole;
mol is mole;
MOM is methoxymethyl ether group;
MS (m/z) is mass spectrum peak;
NMR is nuclear magnetic resonance;
Pd/C is palladium on carbon;
Pd(dppf)Cl₂ is [1,1′-bis(diphenylphophino)ferrocene]dichloropalladium(II);
pH is power of hydrogen;
ppm is parts per million;
psi is pounds per square inch;
q is quartet;
rpm is revolutions per minute;
rt is room temperature;
RT is retention time;
RuPhos Pd G3 is (2-dicyclohexylphosphino-2′,6′-diisopropoxy-1,1′-biphenyl)[2-(2′-amino-1,1′-biphenyl)]palladium(II) methanesulfonate (CAS Number: 1445085-77-7);
s is singlet;
SEMCl is 2-(trimethylsilyl)ethoxymethyl chloride;
SEM is 2-(trimethylsilyl)ethoxymethyl;
SFC is supercritical fluid chromatography;
t is triplet;
TBAF is tert-butyl ammonium fluoride;
TFA is trifluoroacetic acid;
THE is tetrahydrofuran;
TLC is thin layer chromatography;
TMSCN is trimethylsilyl cyanide;
TsCl is p-toluenesulfonyl chloride;
μL is microliter; and
μmol is micromole.

The schemes described below are intended to provide a general description of the methodology employed in the preparation of the compounds of the present invention. Some of the compounds of the present invention contain a single chiral center. In the following schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

### General Methods

Unless stated otherwise, the variables in Schemes I-III have the same meanings as defined herein.

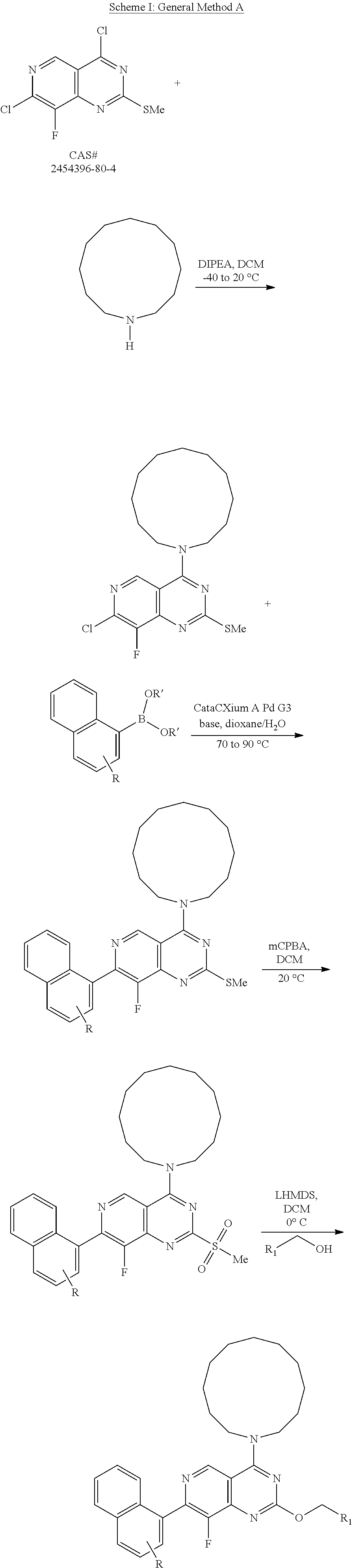

As exemplified in Scheme I, 4,7-dichloro-8-fluoro-2-(methylthio)pyrido[4,3-d]pyrimidine (CAS #: 2454396-80-4) may be treated with an amine in the presence of an effective base (such as DIPEA) in an appropriate solvent (such as DCM) to provide an adduct via a SnAr reaction at the 4-chloro position. A Suzuki reaction at the 7-chloro position brings in the naphthol group using a palladium catalyst (such as CataCXium A Pd G3) and a base (such as K₂CO₃ or K₃PO₄) in a suitable solvent such as dioxane/water. Oxidation of the 2-thiomethyl group to the sulfone can be done using an oxidant (such as mCPBA) in a solvent (such as DCM). The resulting sulfone group can be displaced by an alcohol nucleophile (such as ((2R,7aS)-2-fluorotetrahydro-1H-pyrrolizin-7a(5H)-yl)methanol, CAS #2097518-76-6) using a suitable base (such as LHMDS) in a suitable solvent (such as DCM). In some cases, the penultimate intermediate may contain protecting groups, which may be removed by additional steps in the synthetic sequence using conditions known in the art (March's Advanced Organic Chemistry: Reactions, Mechanisms, and Structure 8th Edition or Protecting Groups, 10 Georg Thieme Verlag, 1994). Compounds at every step may be purified by standard techniques, such as column chromatography, crystallization, reverse phase HPLC or SFC.

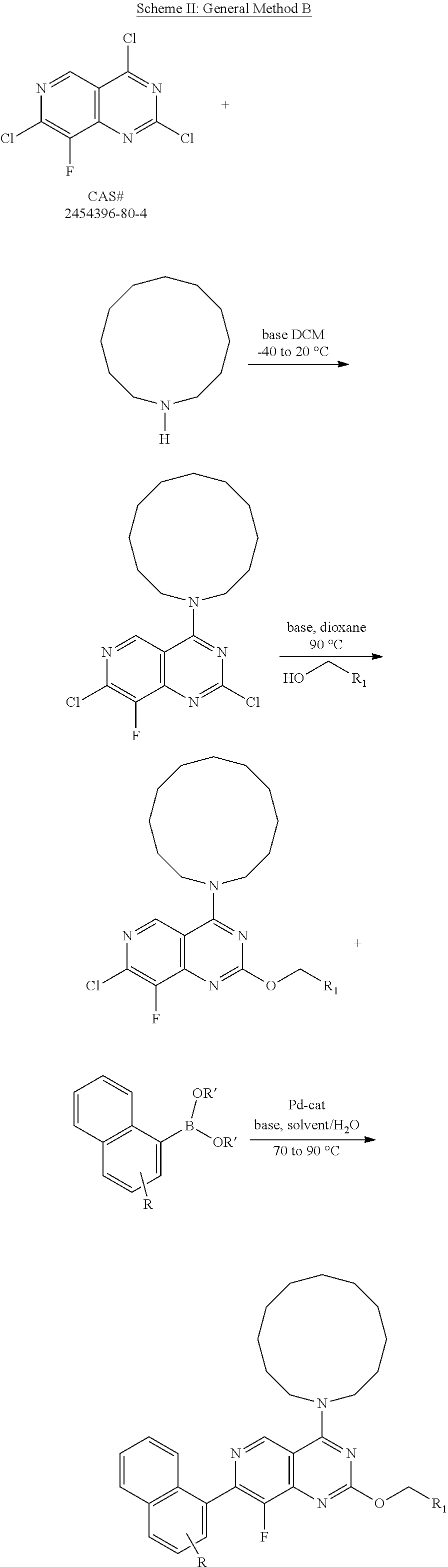

As exemplified in Scheme II, 2,4,7-trichloro-8-fluoropyrido[4,3-d]pyrimidine (CAS #2454396-80-4) may be treated with an amine in the presence of an effective base (such as DIPEA) in an appropriate solvent (such as DCM) to provide an adduct via a SnAr reaction at the 4-chloro position. A second SnAr reaction at the 2-chloro position brings in an alcohol nucleophile (such as ((2R,7aS)-2-fluorotetrahydro-1H-pyrrolizin-7a(5H)-yl)methanol, CAS #2097518-76-6) in the presence of a base (such as DIPEA) in an appropriate solvent (such as 1,4-dioxane) at elevated temperature (such as 90° C.). A Suzuki reaction at the 7-chloro position brings in the naphthol group using a palladium catalyst (such as Pd(OAc)₂/dppf) and a base (such as NaOH) in a suitable solvent such as CH₃CN/water.

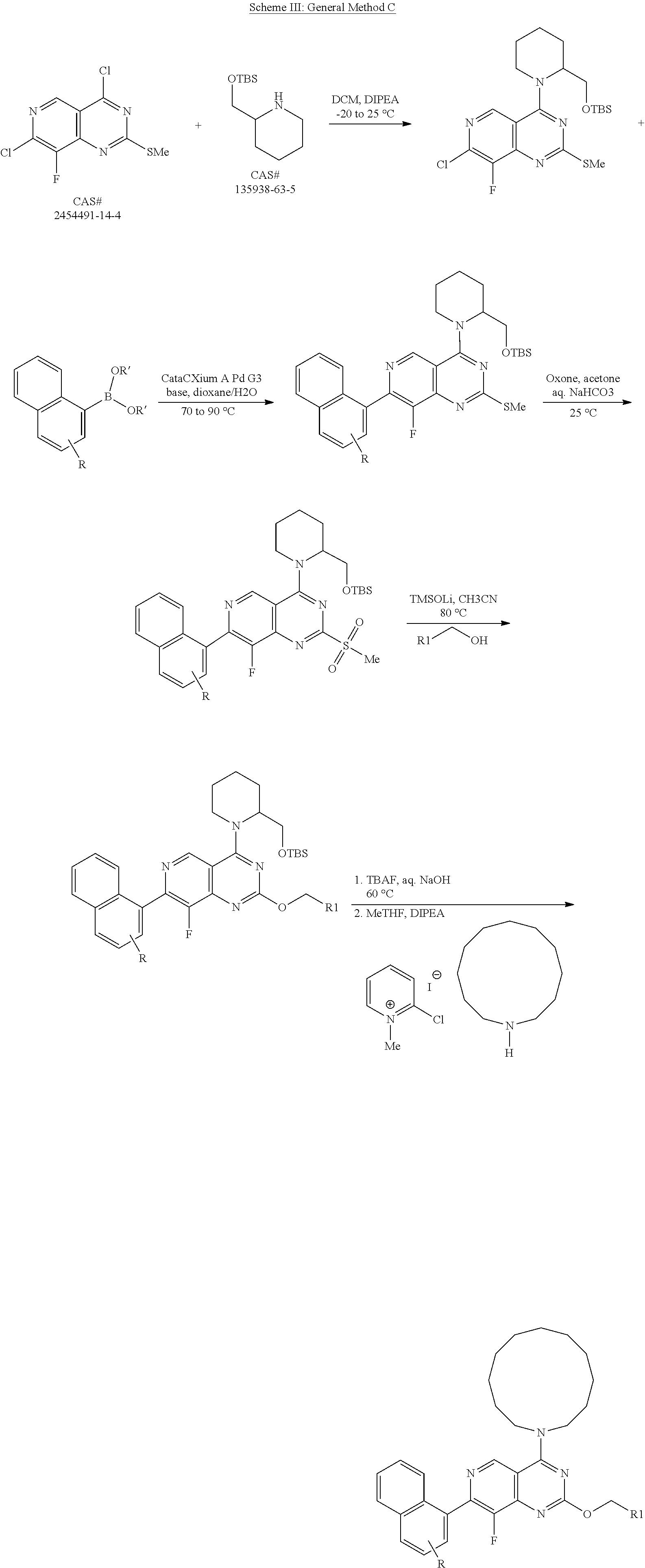

As exemplified in Scheme III, 4,7-dichloro-8-fluoro-2-(methylthio)pyrido[4,3-d]pyrimidine (CAS #2454491-14-4) may be treated with a placeholder amine (such as 2-(((tert-butyltyldimethylsilyl)oxy)methyl)piperidine) in the presence of an effective base (such as DIPEA) in an appropriate solvent (such as DCM) to provide an adduct via a SnAr reaction at the 4-chloro position. A Suzuki reaction at the 7-chloro position brings in the naphthol group using a palladium catalyst (such as CataCXium Pd G3) and a base (such as K₃PO₄) in a suitable solvent (such as THE/water). Oxidation of the thiomethyl group to the sulfone can be accomplished using an oxidant (such as buffered Oxone) in a solvent (such as acetone/water). A second SnAr reaction at the 2-sulfonyl position brings in an alcohol nucleophile (such as ((2R,7aS)-2-fluorotetrahydro-1H-pyrrolizin-7a(5H)-yl)methanol) in the presence of a base (such as LiOTMS) in an appropriate solvent (such as CH₃CN) at elevated temperature (such as 80° C.). The placeholder amine can be removed using a base (such as NaOH) in the presence of a fluoride source (such as TBAF) at elevated temperature (such as 60° C.). The desired C4 amine is then added in the presence of 2-chloro-1-methylpyridinium iodide and an effective base (such as DIPEA) in an appropriate solvent (such as 2-MeTHF) to provide an adduct via a SnAr reaction at the 4-oxo position.

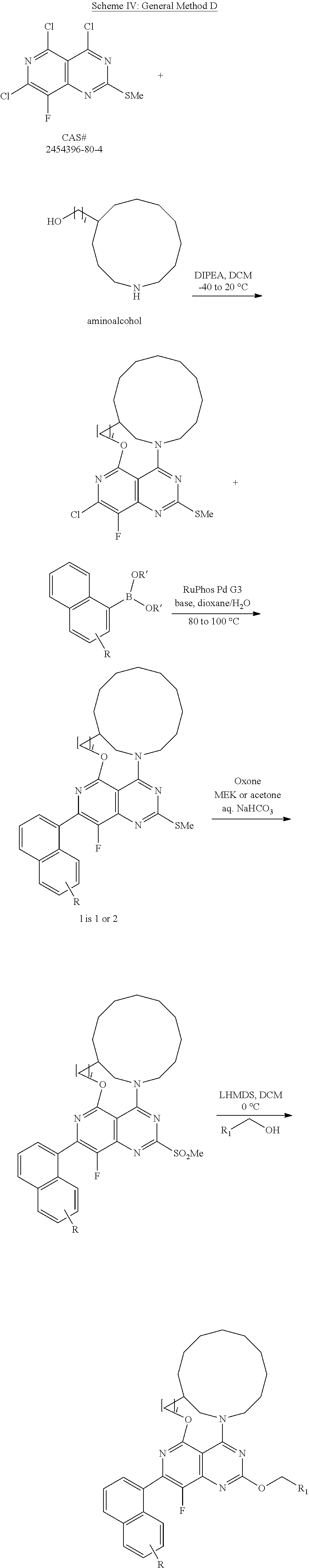

As exemplified in Scheme IV, 4,5,7-trichloro-8-fluoro-2-(methylthio)pyrido[4,3-d]pyrimidine may be treated with an aminoalcohol (cyclic or acyclic) in the presence of an effective base (such as DIPEA) in an appropriate solvent (such as DCM) to provide an adduct via a SnAr reaction at the 4-chloro position. A Suzuki reaction at the 7-chloro position brings in the naphthol or naphthyl group using a palladium catalyst (such as RuPhos Pd G3) and a base (such as K₂CO₃ or K₃PO₄) in a suitable solvent such as dioxane/water. Oxidation of the 2-thiomethyl group to the sulfone can be done using an oxidant (such as Oxone) in a buffered aqueous solvent containing NaHCO₃ and either acetone or methylethylketone. The resulting sulfone group can be displaced by an alcohol nucleophile (such as ((2R,7aS)-2-fluorotetrahydro-1H-pyrrolizin-7a(5H)-yl)methanol, CAS #2097518-76-6) using a suitable base (such as LHMDS or LiOTMS) in a suitable solvent (such as CH₃CN). In some cases, the penultimate intermediate may contain protecting groups, which may be removed by additional steps in the synthetic sequence using conditions known in the art (March's Advanced Organic Chemistry: Reactions, Mechanisms, and Structure 8th Edition or Protecting Groups, 10 Georg Thieme Verlag, 1994). Compounds at every step may be purified by standard techniques, such as column chromatography, crystallization, reverse phase HPLC or SFC.

Variable R¹ in Schemes I-III is the same as defined in the embodiments E1-E31 herein.

Variable R in Schemes I-III represents one to four substituents selected from the group consisting of —OH, halogen, CN, C₁-C₃ alkyl, C₁-C₃ fluoroalkyl, C₁-C₃ alkoxy, and C₂-C₃ alkynyl, as defined in the embodiments and claims herein.

Variable R′ in Schemes I-III can be but is not limited to H, or C₁-C₅ alkyl, wherein the two R′ may join to form a ring, R′ is not part of definitions in claims herein.

The amine

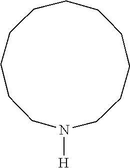 [SMILES: C1CCCCCNCCCCC1]

as provided in Schemes I-III is a generic structure corresponding to the definition of R² as defined in the embodiments E1-E31, wherein R² is:

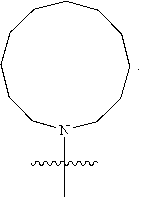 [SMILES: *N1CCCCCCCCCCC1]

Variable R¹ in Scheme IV is the same as defined in the embodiments E32-E60 herein.

Variable R in Scheme IV represents one to four substituents selected from the group consisting of —OH, halogen, CN, C₁-C₃ alkyl, C₁-C₃ fluoroalkyl, C₁-C₃ alkoxy, and C₂-C₃ alkynyl, as defined in the embodiments and claims herein.

Variable R′ in Scheme IV can be but is not limited to H, or C₁-C₅ alkyl, wherein the two R′ may join to form a ring, R′ is not part of definitions in claims herein.

The aminoalcohol in Scheme IV is a representative generic moiety that can form desired tetracyclic ring structure as defined in embodiments such as E32 (wherein R² and R⁵ are optionally taken together to form a 4-8 membered heterocycloalkyl) and E39. The aminoalcohol can be an acyclic moiety that can from desired tricyclic ring structure as defined in embodiments such as E32 when R² and R⁵ are not taken together to form a 4-8 membered heterocycloalkyl.

The synthetic intermediates as generally defined in the above schemes are useful for preparing compounds of the invention and synthesis of such non-commercial intermediates are provided as further aspects of this invention.

### Preparations

### Preparation 1: (1R,5R,6R)-3-azabicyclo[3.2.1]octan-6-ol

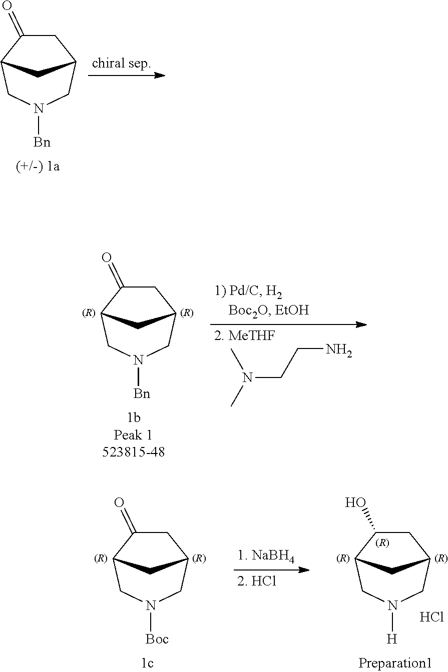

Chiral separation of 1a (700 g, 3.2 mol), prepared as described in J. Med. Chem. 2012, 55(10), 4605, was conducted using chiral SFC (AS-H column, mobile phase of 95/5 CO₂/EtOH, 2 mL inj. volume, 2.5 mL/min flow rate, 35° C.). Retention times using the prep. AS-H column were 3.01 min. for peak 1 and 3.70 min. for peak 2 with baseline resolution. The peak 1 material had an er ratio of 100/0. The peak 1 material (1 b, 294 g, 1.37 mol) from the chiral separation was dissolved in EtOH (3.2 L) and Boc₂O (486 g, 2.23 mol) was added followed by 10% Pd/C (10 g). The reaction was stirred at 25° C. under an atmosphere of H₂ (60 psi) for 18 h. Then, fresh Pd/C (5 g) was added and stirring under H₂ (60 psi) was continued for 30 min. HPLC showed that all the benzyl group had been removed. The catalyst was filtered off and most of the EtOH was removed in vacuo to afford a yellow oil. Heptane (500 mL) was added and the mixture was coevaporated to remove as much EtOH as possible. The yellow residue was dissolved in 2-MeTHF (1.8 L) and cooled in an ice water bath. When the internal temperature was below 5° C., N,N-dimethylethylenediamine (105 mL, 0.956 mol) was added to quench the excess Boc₂O. After the addition, the reaction was allowed to warm to room temperature and stir for 45 min. The reaction was re-cooled in an ice bath and 1 N HCl (800 mL) was added to pH 2 while holding the internal temperature below 20° C. The layers were separated and the aq. layer was extracted with 2-MeTHF (1×300 mL). The combined organic extract was washed with sat. NaHCO₃, brine and dried over MgSO₄. The solvent was removed to give a yellow oil. Coevaporation with heptane (250 mL) gave 286 g of a crude oil with 89% purity by HPLC. Heptane (1.1 L) was added to the oil and the mixture was cooled in an ice water bath. When the internal temperature reached 9° C., the mixture turned cloudy. A few seed crystals were added and the mixture was allowed to slowly warm to room temperature overnight. The solids that formed were collected by filtration washing with a small amount of cold heptane. After suction drying tert-butyl (1R,5R)-6-oxo-3-azabicyclo[3.2.1]octane-3-carboxylate (1c, 237 g, 77%, 97.4% pure by HPLC) was obtained as a white solid. A solution of 1c (5.0 g, 22 mmol) in MeOH (100 mL) was cooled in an ice bath. NaBH₄ (924 mg, 24.4 mmol) was added and the reaction was stirred at for 0° C. 10 min. The ice bath was removed and the reaction was stirred at rt for 1 h. The methanol was removed under vacuum, and the resulting residue was partition between sat. aq. NaHCO₃ and EtOAc. The aqueous layer was further extracted with EtOAc and the combine organic extract was dried over Na₂SO₄ and concentrated to give 5.5 g of the Boc-protected alcohol a white solid. The yield was slightly over 100% due to the presence of residual EtOAc in the sample. ¹H NMR (400 MHz, CHLOROFORM-d) δ=4.30-4.18 (m, 2H), 3.81 (br d, J=12.2 Hz, 1H), 2.93 (br d, J=12.5 Hz, 1H), 2.87 (dd, J=1.2, 13.2 Hz, 1H), 2.29 (ddd, J=7.0, 10.6, 13.9 Hz, 1H), 2.14 (br s, 2H), 1.69-1.62 (m, 1H), 1.59-1.53 (m, 1H), 1.48 (s, 9H), 1.46-1.41 (m, 1H), 1.15 (td, J=2.8, 14.0 Hz, 1H). To a solution the Boc-protected alcohol (5.0 g, 22 mmol) in DCM (5 mL) was added HCl in dioxane (25 mL of 4 N, 100 mmol). Gas evolution was immediate. The reaction was stirred at rt for 5 min and a white precipitate formed. After an additional 1 h, the reaction was diluted with heptane, and the white solid was collected by filtration. The solid was dried overnight under high vacuum to give (1R,5R,6R)-3-azabicyclo[3.2.1]octan-6-ol HCl salt, Preparation 1 (3.5 g, 97%) as a white powder. ¹H NMR (400 MHz, D₂O) δ=4.63-4.54 (m, 1H), 3.48 (dd, J=2.6, 12.6 Hz, 1H), 3.24 (s, 2H), 3.16 (br d, J=12.7 Hz, 1H), 2.50-2.38 (m, 3H), 1.77 (d, J=2.2 Hz, 2H), 1.53-1.45 (m, 1H); MS: [M+H]⁺ 128.1.

### Preparation 2-(+) and Preparation 2-(−): tert-butyltyl-(1R*,5R*,6R*)-6-hydroxy-8-oxa-3-azabicyclo[3.2.1]octane-3-carboxylate and tert-butyltyl-(1S*,5S*,6S*)-6-hydroxy-8-oxa-3-azabicyclo[3.2.1]octane-3-carboxylate

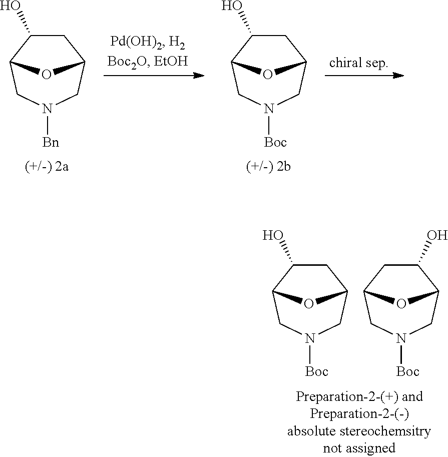

A racemic mixture of (+/−) 2a was prepared as described in US patent 2013/0079321. Treatment of the optical mixture of (+/−) 2a (3.80 g, 16.6 mmol) with Boc₂O (5.7 g, 26 mmol) and Pd(OH)₂ on carbon (4 g) in EtOH (40 mL) at 50° C. under 30 psi H₂ overnight. The reaction mixture was filtered through a pad of celite and the filtrate was concentrated. The residue was suspended and stirred in petroleum ether (30 mL) for 1 hr and filtered. The filtered cake was dried in vacuum to afford a racemic mixture of (+/−) 2b (3.4 g, 87%) as white solid. ¹H NMR (400 MHz, CDCl₃) δ=4.32 (ddd, J=4.1, 6.0, 10.6 Hz, 1H), 4.14 (br s, 1H), 4.05-3.87 (m, 2H), 3.56 (br s, 1H), 3.08 (br s, 2H), 2.96 (br d, J=12.9 Hz, 1H), 2.38 (ddd, J=7.9, 10.8, 12.4 Hz, 1H), 1.36 (s, 9H), 1.21-1.13 (m, 1H), MS: 130 [M+H-Boc]⁺.

The racemic mixture of (+/−) 2b was resolved using chiral SFC as follows: 850 mg of (+/−) 2b was separated into its enantiomers using chiral SFC (Chiralpak IG SFC Sum 21×250 mm column, mobile phase of 90/10 CO₂/MeOH isocratic, 120 bar, 70 mL/min flow rate).
Peak 1=Preparation 2-(+): [α]_{D}²² +10.7 (c 0.3, MeOH), 392 mg, >99.0% ee.
Peak 2=Preparation 2-(−): [α]_{D}²² −27.1 (c 0.1, MeOH), 294 mg, 98% ee.

### Preparation 3: (S)-2-(1-acetylpiperazin-2-yl)acetonitrile

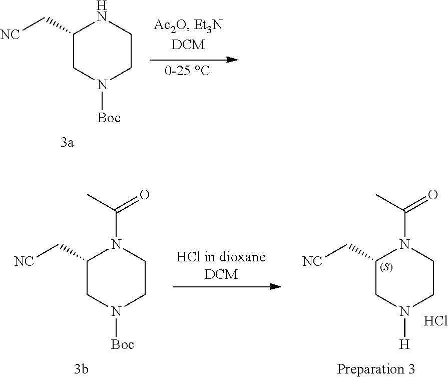

Compound 3a was prepared as described in J. Med. Chem. 2020, 63(13), 6679. To compound 3a (4.9 g, 22 mmol) and Et₃N (3.3 g, 33 mmol) in DCM (50 mL) was added acetic anhydride (2.44 g, 23.9 mmol) at 0° C. After stirring for 1 h at 20° C., LCMS showed product formation. The mixture was washed with sat. aq. NaHCO₃ (100 mL×2) and the organic phase was washed with sat. aq. citric acid (100 mL×2), brine (100 mL), and dried over Na₂SO₄. After filtration and concentration, 3b (6.2 g) was obtained as yellow oil. ¹H NMR (400 MHz, DMSO-d₆) δ 4.66 (s, 1H), 3.87 (d, J=13.8 Hz, 2H), 3.47 (d, J=195.2 Hz, 1H), 3.11-3.02 (m, 2H), 2.75 (s, 3H), 2.05 (s, 3H), 1.43 (s, 9H); MS: [M+H+Na]⁺ 290. To a solution of 3b (5.80 g, 21.7 mmol) in DCM (40 mL) was added HCl in dioxane (15 mL, 4 M) and the mixture was stirred for 5 h at 25° C. LCMS showed product formation and a white solid precipitated from solution. The solid was filtered and dried to afford (S)-2-(1-acetylpiperazin-2-yl)acetonitrile HCl salt, Preparation 3 (5 g, crude yield). ¹H NMR (400 MHz, DMSO-d₆) δ 9.72 (d, J=67.0 Hz, 2H), 4.83 (d, J=149.1 Hz, 1H), 4.20 (dd, J=226.7, 14.2 Hz, 1H), 3.66-3.45 (m, 1H), 3.40-2.74 (m, 6H), 2.11 (d, J=26.4 Hz, 3H), MS: 168 [M+H]⁺.

### Preparation 4: (S)-2-(3-methylpyrrolidin-3-yl)acetonitrile

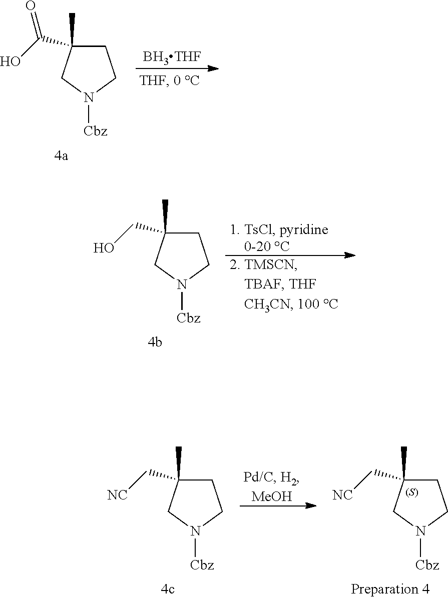

(R)-1-((benzyloxy)carbonyl)-3-methylpyrrolidine-3-carboxylic acid (4a, 0.700 g, 2.66 mmol) was dissolved in THE (10 mL). BH₃-THF (7.98 mL of 1 M, 7.98 mmol) was added dropwise at 0° C. Then, the reaction was warmed to 25° C. and stirred at 25° C. for 2 h. LCMS analysis showed 4a was consumed and the desired alcohol 4b was observed. The reaction was quenched via dropwise addition of MeOH (10 mL). The resulting solution was concentrated and purified using flash chromatography eluting with a gradient of 0-40% EtOAc in petroleum ether to provide 4b (600 mg, 90%) as a colorless oil. ¹H NMR (DMSO-d₆, 400 MHz) δ 7.41-7.21 (m, 5H), 5.05 (d, 2H, J=3.5 Hz), 3.59 (br d, 1H, J=5.5 Hz), 3.2-3.4 (m, 4H), 3.12-2.93 (m, 1H), 1.94-1.73 (m, 1H), 1.65-1.44 (m, 2H), 0.98 (s, 3H), MS: 250.1 [M+H]⁺. To a solution of 4b (600 mg, 2.41 mmol) in pyridine (20 mL) was added TsCl (551 mg, 2.89 mmol) portionwise at 0° C. Then, the solution was warmed to 25° C. and stirred for 24 h. LCMS analysis showed that 4b was consumed, and the desired product was observed. The pyridine was removed in vacuo and the resulting residue was diluted with EtOAc (50 mL). The organic layer was washed with 1 N HCl (50 mL). The organic layer was dried over anhydrous Na₂SO₄, filtered and concentrated to afford (R)-3-methyl-3-((tosyloxy)methyl)pyrrolidine-1-carboxylate (1.9 g) as a crude yellow solid, which was used directly in next step. MS: 404.1 [M+H-56]⁺. Crude (R)-3-methyl-3-((tosyloxy)methyl)pyrrolidine-1-carboxylate (2.41 mmol) from above was dissolved in CH₃CN (30 mL). TMSCN (836 mg, 8.43 mmol) and TBAF (8.43 mL of 1.0 M in THF, 8.43 mmol) were added. The resulting solution was heated at 100° C. for 16 h. LCMS analysis showed that the tosylate was consumed and the desired product was observed. The reaction mixture was diluted with H₂O (100 mL) and extracted with EtOAc (3×100 mL). The combined organic extract was dried over anhydrous Na₂SO₄, filtered and concentrated. The residue was purified using flash chromatography eluting with a gradient of 0-60% EtOAc in petroleum ether to afford 4c (620 mg, 100%) as a colorless oil. ¹H NMR (DMSO-d₆, 400 MHz) δ 7.46-7.25 (m, 5H), 5.06 (d, 2H, J=2.9 Hz), 3.55-3.34 (m, 2H), 3.37-3.12 (m, 2H), 2.76-2.64 (m, 2H), 1.98-1.67 (m, 2H), 1.14 (d, 3H, J=2.2 Hz). The NMR shows evidence of restricted rotation around the N-Cbz bond, MS: 281.1 [M+H+Na]⁺. To a solution of 4c (200 mg, 0.774 mmol) in MeOH (10 mL) was added 10% Pd/C (41 mg). The reaction was stirred at 25° C. under H₂ (1 atm) for 4 h. LCMS analysis showed that 4c was consumed and the product was observed. The reaction mixture was filtered and the filtrate was concentrated to afford (S)-2-(3-methylpyrrolidin-3-yl)acetonitrile, Preparation 4 (120 mg) as a colorless oil. ¹H NMR (CDCl₃, 400 MHz) δ 3.83 (br s, 1H), 3.07 (t, 1H, J=7.2 Hz), 2.86 (d, 1H, J=11.0 Hz), 2.75 (d, 1H, J=11.0 Hz), 2.43 (s, 2H), 1.6-1.8 (m, 2H), 1.26 (s, 3H), MS: 125.2 [M+H]⁺.

### Preparation 5: (S)-2-(3-methylpiperidin-3-yl)acetonitrile

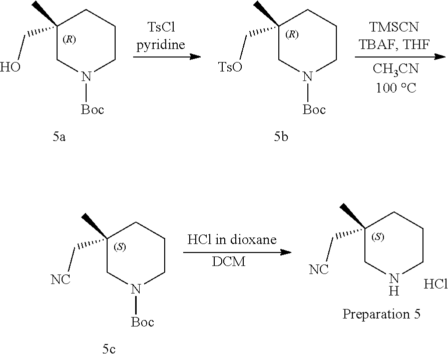

Compound 5a tert-butyltyl (R)-3-(hydroxymethyl)-3-methylpiperidine-1-carboxylate (900 mg, 3.92 mmol) was dissolved in pyridine (30 mL), cooled to 0° C. and TsCl (1.5 g, 7.8 mmol) was added. The reaction mixture was warmed to 25° C. and stirred for 24 h. LCMS analysis showed that 5a was consumed and 5b was observed. The pyridine was removed in vacuo and the residue dissolved in EtOAc (50 mL). The organic layer was washed with aq. 1 N HCl (2×30 mL). The organic phase was dried over anhydrous Na₂SO₄, filtered and concentrated to afford 5b (550 mg, 37%) as a gum. ¹H NMR (CDCl₃, 400 MHz) δ 7.78 (d, 2H, J=8.2 Hz), 7.35 (d, 2H, J=8.1 Hz), 3.92-3.65 (m, 2H), 3.36 (br s, 1H), 3.31-3.18 (m, 2H), 3.04 (br d, 1H, J=13.2 Hz, 2H), 2.46 (s, 3H), 1.70-1.54 (m, 1H), 1.52-1.46 (m, 1H), 1.44 (s, 9H), 1.33-1.23 (m, 2H), 0.86 (s, 3H), MS: 328.1 [M+H-56 tert-butyl]⁺. To a solution of 5b (450 mg, 1.17 mmol) in CH₃CN (20 mL) was added TMSCN (233 mg, 2.35 mmol) and TBAF (1.17 mL of 1 M in THF, 1.17 mmol). The solution was heated at 100° C. for 24 h. LCMS analysis showed that 5b was consumed and 5c was observed. The CH₃CN was removed in vacuo and the residue purified using flash chromatography using a gradient of 0-15% EtOAc in petroleum ether, to give tert-butyltyl (S)-3-(cyanomethyl)-3-methylpiperidine-1-carboxylate, 5c (240 mg, 70%) as colorless oil. ¹H NMR (400 MHz, CDCl₃) δ 3.64 (s, 1H), 3.45 (s, 1H), 3.11 (s, 1H), 2.99 (d, J=13.4 Hz, 1H), 2.31 (s, 2H), 1.76-1.51 (m, 4H), 1.46 (s, 9H), 1.10 (s, 3H). To a solution of 5c (200 mg, 0.839 mmol) in DCM (5 mL) was added HCl in dioxane (1.0 mL of 4 N, 4.0 mmol). Then the reaction was stirred at 25° C. for 2 h. LCMS analysis showed that 5c was consumed and Preparation 5 was observed. The reaction was concentrated to afford (S)-2-(3-methylpiperidin-3-yl)acetonitrile HCl salt, Preparation 5 (116 mg, 79%) as colorless oil which was used for next step without further purification. ¹H NMR (400 MHz, DMSO) δ=9.11 (br s, 1H), 8.81 (br s, 1H), 3.57 (s, 3H), 3.38 (s, 2H), 2.91 (s, 2H), 2.75 (d, J=4.7 Hz, 1H), 1.73-1.64 (m, 1H), 1.58-1.47 (m, 1H), 1.11 (s, 3H), MS: 139 [M+H]⁺.

### Preparation 6: rac-(4-fluoropyrrolidin-3-yl)acetonitrile

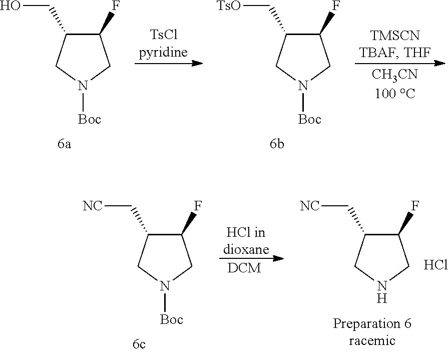

To a solution of 6a (750 mg, 3.42 mmol) in pyridine (30 mL) was added TsCl (1.30 g, 6.84 mmol) at 0° C. Then the reaction mixture was warmed to 25° C. and stirred for 24 h. LCMS analysis showed that 6a was consumed and 6b was observed. The pyridine was removed in vacuo and the residue diluted with EtOAc (50 mL). The organic layer was washed with aq. 1 N HCl (2×30 mL). The combined organic phase was dried over anhydrous Na₂SO₄, filtered and concentrated to get 6b (670 mg, 52%) as colorless gum. ¹H NMR (400 MHz, DMSO) δ 7.81 (d, J=8.2 Hz, 2H), 7.50 (d, J=8.1 Hz, 2H), 5.05 (d, J=51.8 Hz, 1H), 4.03 (d, J=7.1 Hz, 2H), 3.50-3.38 (m, 3H), 3.10 (dd, J=11.4, 2.6 Hz, 1H), 2.71-3.38 (m, 1H), 2.43 (s, 3H), 1.38 (s, 9H). MS: 318.1 [M+H-56 tert-butyl]⁺. To a solution of 6b (670 mg, 1.79 mmol) in CH₃CN (10 mL) was added TMSCN (356 mg, 3.59 mmol) and TBAF (1.79 mL of 1 M in THF, 1.79 mmol). The solution was heated at 100° C. for 3 h. LCMS analysis showed that 6b was consumed and 6c was observed. The solvents were removed in vacuo and the residue was purified using flash chromatography eluting with a gradient of 0-30% EtOAc in petroleum ether. After concentration of the pure fractions, 6c (330 mg, 69%) was obtained as a colorless gum. ¹H NMR (400 MHz, CDCl₃) δ 4.95 (dd, J=52.0, 17.4 Hz, 1H), 3.70-3.51 (m, 3H), 3.29 (s, 1H), 2.68 (d, J=7.7 Hz, 1H), 2.38 (dd, J=26.2, 11.9 Hz, 2H), 1.40 (s, 9H), MS: 173.1 [M+H-56 tert-butyl]⁺. To a solution of 6c (300 mg, 1.13 mmol) in DCM (25 mL) was added HCl in dioxane (6.57 mL of 4 N, 26.3 mmol). The mixture was stirred at 25° C. for 24 h. LCMS analysis showed that 6c had been consumed and Preparation 6 was detected. The solvents were removed to afford rac-(4-fluoropyrrolidin-3-yl)acetonitrile hydrochloride, Preparation 6 (185 mg, 100%) as a colorless oil. ¹H NMR (400 MHz, DMSO) δ 10.27-9.66 (m, 2H), 5.31-5.10 (m, 1H), 3.60-3.36 (m, 3H), 3.08 (d, J=5.7 Hz, 1H), 2.96-2.70 (m, 2H), 2.34-2.16 (m, 1H). MS: 129.1 [M+H]⁺.

### Preparation 7: trimethyl[2-({[4-(4,4,5,5-tetramethyl-1,3,2-dioxaborolan-2-yl)-5-{[tri(propan-2-yl)silyl]ethynyl}naphthalen-2-yl]oxy}methoxy)ethyl]silane

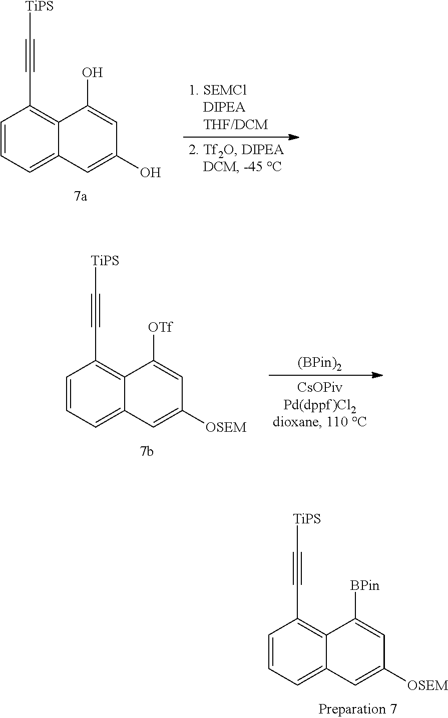

Compound 7a was prepared as described in WO2021041671. To a mixture of 7a (30.2 g, 88.7 mmol) and DIPEA (17.2 g, 133 mmol) in DCM/THF (500 mL/50 mL) was added SEMCl (14.8 mg, 88.7 mmol). The mixture was stirred at 20° C. for 16 h. The reaction was concentrated and the residue was purified by flash chromatography eluting with 5% EtOAc in petroleum ether to afford the SEM protected intermediate as yellow oil (21.5 g, 51%). ¹H NMR (400 MHz, CDCl₃) δ 9.24 (s, 1H), 7.71-7.66 (m, 1H), 7.48 (dd, J=7.1, 1.1 Hz, 1H), 7.30 (dd, J=8.2, 7.3 Hz, 1H), 6.98 (d, J=2.4 Hz, 1H), 6.76 (d, J=2.4 Hz, 1H), 5.31 (s, 2H), 3.84-3.71 (m, 2H), 1.21-1.15 (m, 21H), 1.00-0.94 (m, 2H), −0.00 (s, 9H), MS: 471 [M+H]⁺. The SEM protected intermediate (21.5 g, 45.7 mmol) was dissolved in DCM (300 mL). DIPEA (11.8 g, 91.3 mmol) was added and the reaction was cooled to −45° C. Tf₂O (19.3 g, 68.5 mmol) was added dropwise and the reaction was stirred at −45° C. for 1 h. Analysis by TLC (5% EtOAc in petroleum ether) indicated a complete reaction. The reaction mixture was poured into H₂O (200 mL) and the aqueous layer was extracted with DCM (2×200 mL). The combined organic extract was washed with brine (100 mL), dried over anhydrous sodium sulfate, and concentrated. The residue was purified by flash chromatography eluting with 5% EtOAc in petroleum ether to afford 7b (26.8 g, 97%) as an orange oil. ¹H NMR (400 MHz, CDCl₃) δ 7.73 (t, J=6.9 Hz, 2H), 7.45-7.40 (m, 2H), 7.30 (d, J=2.2 Hz, 1H), 5.33 (s, 2H), 3.86-3.74 (m, 2H), 1.16 (d, J=5.9 Hz, 21H), 1.01-0.93 (m, 2H), 0.00 (s, 9H). To a mixture of 7b (6.00 g, 9.95 mmol), was added bis(pinacolato)diboron (5.05 g, 19.9 mmol) and cesium pivalate (4.66 g, 19.9 mmol) in dioxane (100 mL). Pd(dppf)Cl₂ (728 mg, 0.995 mmol) was added and the mixture was stirred at 110° C. for 48 h under N₂. LCMS analysis showed product formation. The mixture was cooled and concentrated. The residue was purified by flash chromatography eluting with a gradient of 10-30% DCM in petroleum ether to afford trimethyl[2-({[4-(4,4,5,5-tetramethyl-1,3,2-dioxaborolan-2-yl)-5-{[tri(propan-2-yl)silyl]ethynyl}naphthalen-2-yl]oxy}methoxy)ethyl]silane, Preparation 7 (2.58 g, 45%) as an orange oil. ¹H NMR (400 MHz, CDCl₃) δ 7.71-7.66 (m, 2H), 7.46 (d, J=2.5 Hz, 1H), 7.39 (d, J=2.5 Hz, 1H), 7.35 (d, J=7.5 Hz, 1H), 5.32 (s, 2H), 3.87-3.69 (m, 2H), 1.43 (s, 12H), 1.15 (s, 21H), 1.01-0.95 (m, 2H), 0.00 (s, 9H), MS: 581 [M+H]⁺.

### Preparation 8: (7,8-difluoro-3-hydroxynaphthalen-1-yl)boronic acid

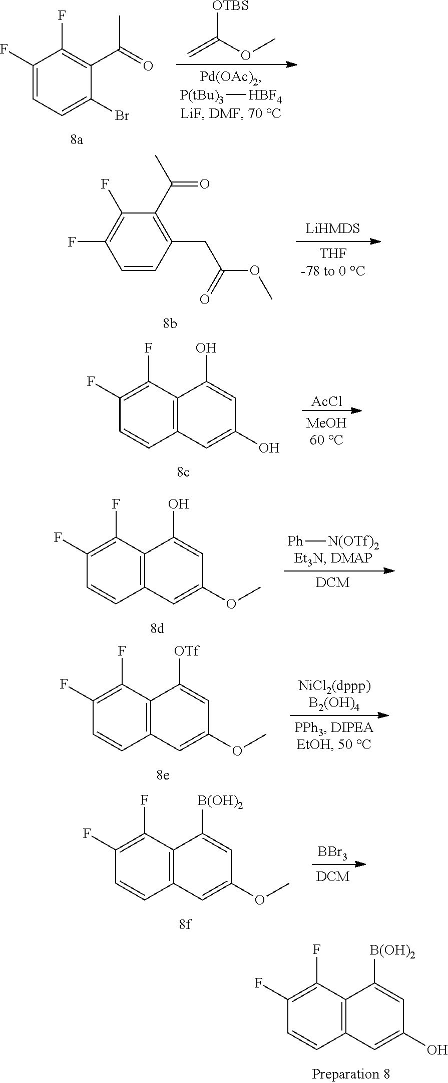

Compound 1-(6-bromo-2,3-difluorophenyl)ethan-1-one (8a), (7.39 g, 31.4 mmol) was dissolved in DMF (79 mL). Pd(OAc)₂ (706 mg, 3.14 mmol), (tert-Bu)₃P-HBF₄ (1.82 g, 6.29 mmol), and LiF (4.89 mg, 189 mmol) were added followed by tert-butyltyl((1-methoxyvinyl)oxy)dimethylsilane (18.4 g, 97.5 mmol). N₂ was bubbled through the mixture for 10 min and the reaction was heated at 70° C. for 30 min. The reaction was cooled to rt and H₂O (50 mL) and EtOAc (100 mL) were added. The layers were separated, and the aqueous layer was extracted with another portion of EtOAc (100 mL). the combined organic extract was washed with brine (5×50 mL) and concentrated. The residue was dissolved in Et₂O (100 mL) and heptane (200 mL) added followed by removal of the heptane to remove the residual DMF via azeotrope. The resulting residue was purified using flash chromatography eluting with a gradient of 0-100% EtOAc in heptane to afford 8b (6.24 g, 87%) as an oil. ¹H NMR (400 MHz, CDCl₃) δ 7.18 (dt, J=9.5, 8.3 Hz, 1H), 6.97 (ddd, J=8.4, 4.4, 1.7 Hz, 1H), 3.77 (s, 2H), 3.68 (s, 3H), 2.62 (d, J=3.8 Hz, 3H). To a solution of 8b (6.24 g, 27.3 mmol) in THE (273 mL) at −78° C. was added LHMDS (49.2 mL of 1 M in THF, 49.2 mmol) dropwise. The reaction turned red and was stirred for 5 min after which the −78° C. bath was replaced with an ice bath. After 15 min, the reaction was complete based on LCMS analysis and it was quenched by the addition of 2M HCl (100 mL). EtOAc (100 mL) was added and stirring was continued for several min. The layers were separated and the aqueous layer was extracted with EtOAc (3×50 mL). The combined organic extract was washed with brine (50 mL), and dried over sodium sulfate to afford 8c (4.91 g, 91%) as a red solid which was used in the next step without further purification. A solution of MeOH (125 mL) was cooled to 0° C. and acetyl chloride (37.3 g, 476 mmol) was added dropwise. After a slight exotherm, the reaction was allowed to reach 0° C. again. Then, a solution of 8c (4.91 g, 25.0 mmol) in MeOH (50 mL) was added. The reaction was fitted with a reflux condenser and heated at 60° C. for 6.5 h. The reaction was concentrated and purified by flash chromatography using a gradient of 0-50% EtOAc in heptane to afford 8d (3.78 g, 72%) as a beige solid. ¹H NMR (400 MHz, CDCl₃) δ 7.43 (ddd, J=9.2, 4.7, 1.8 Hz, 1H), 7.29-7.21 (m, 1H), 6.74-6.69 (m, 2H), 6.63 (d, J=22.8 Hz, 1H), 3.88 (s, 3H). To a solution of 8d (3.78 g, 40.2 mmol) in DCM (90 mL) was added Et₃N (3.64 g, 36.0 mmol) N,N-Bis(trifluoromethylsulfonyl)aniline (7.71 g, 21.6 mmol) and DMAP (110 mg, 0.899 mmol). The reaction was stirred at rt for 8 h. Additional N,N-bis(trifluoromethylsulfonyl)aniline (1.29 g, 3.60 mmol) was added and the reaction was allowed to stir for another 11 h. The mixture was transferred to a separatory funnel and washed with 1 N NaOH (50 mL). The DCM layer was dried over Na₂SO₄, filtered, and purified by flash chromatography eluting with a gradient of 0-30% EtOAc in heptane, to give 8e (6.7 g) as a solid that exceeds theoretical yield. Since the NMR showed low purity, the material was re-purified using a 220 g Gold Isco column and eluting with a gradient of 0-30% EtOAc in heptane to afford 8e (5.18 g, 84%) as a beige solid. ¹H NMR (400 MHz, CDCl₃) δ 7.53 (ddd, J=9.2, 4.6, 1.9 Hz, 1H), 7.39 (td, J=9.4, 7.3 Hz, 1H), 7.20 (d, J=2.3 Hz, 1H), 7.14 (t, J=2.1 Hz, 1H), 3.94 (s, 3H), MS: 210 [M+H-SO₂CF₃]⁺. Ethanol (56 mL) and DIPEA (7.91 mL, 45.4 mmol) were added to a flask containing 8e (5.18 g, 15.1 mmol). Tetrahydroxydiborane (2.04 g, 22.7 mmol), 1,3-bis(diphenylphosphino)propane nickel (II) chloride (410 mg, 0.757 mmol), and triphenylphosphine (397 mg, 1.51 mmol) were added. Nitrogen was bubbled through the mixture for 5 min. The reaction was heated to 50° C. for 17 h. LCMS shows one new peak that is more polar but does not ionize. The mixture was diluted with EtOAc and the product was extracted with 1 N NaOH (2×75 mL). The aqueous layer was acidified with 6N HCl to pH=1 and extracted with EtOAc (2×200 mL). The EtOAc was dried over Na₂SO₄ and concentrated to afford (7,8-difluoro-3-hydroxynaphthalen-1-yl)boronic acid, Preparation 8 (3.6 g, 99%) as a white solid. ¹H NMR (400 MHz, CDCl₃) δ=7.50 (ddd, J=1.7, 4.7, 9.0 Hz, 1H), 7.42 (d, J=2.3 Hz, 1H), 7.32 (dt, J=7.9, 9.4 Hz, 1H), 7.14 (t, J=2.1 Hz, 1H), 3.93 (s, 3H), with the B(OH)₂ protons appearing as a very broad peak between 2-3 ppm, depending on concentration, ¹⁹F NMR (376 MHz, CDCl₃) δ=−141.77 (d, J=18 Hz, 1F), −144.32 (d, J=20 Hz, 1F).

### Preparation 9: 4,5,7-trichloro-8-fluoro-2-(methylthio)pyrido[4,3-d]pyrimidine

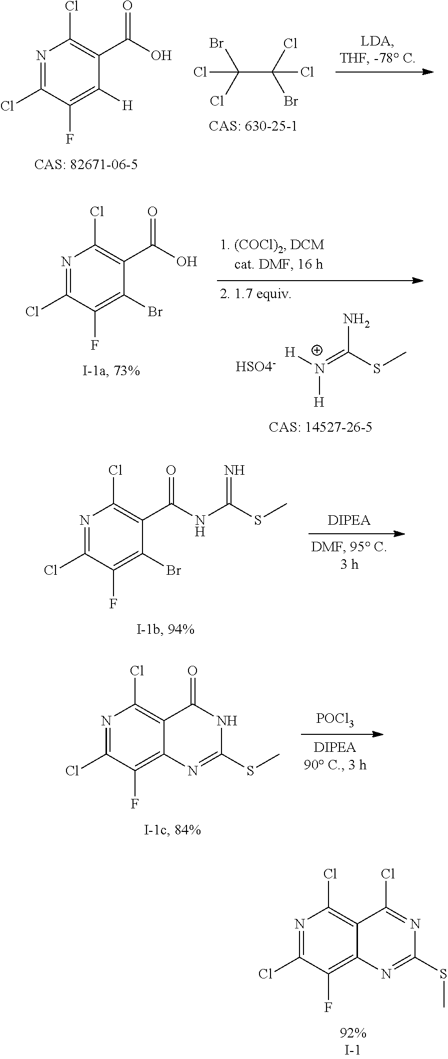

Diisopropyl amine (44.1 mL, 314 mmol) was dissolved in THE (300 mL) and the solution cooled to −78° C. n-BuLi (114 mL of 2.5 M in hexanes, 286 mmol) was added over 15 min. The mixture was stirred for 45 min. and CAS: 82671-06-5 (30 g, 143 mmol) was added as a solution in THE (75 mL) over 6 min. The mixture was stirred for 30 min at −78° C. CAS: 630-25-1 (69.8 g, 214 mmol) was added as a solution in THE (120 mL) over 10 min. The reaction was held at −78° C. for 2 h and the reaction was checked by LCMS. A new peak with M−H=242 (product —CO₂H) was observed (negative ion mode). The mixture was quenched by adding water (120 mL). After stirring for 10 min, at −78° C., the cold bath was removed and 6 N HCl (90 mL) was added. The pH=1 aqueous layer was extracted with EtOAc (×3). The combined organic extract was washed with brine (×2) and dried over MgSO₄. Removal of the solvent afforded a solid that was stirred in heptane (250 mL) for 1 h to remove tetrachloroethylene byproduct. After filtration, the solid was washed with heptane (3×100 mL) and dried to afford 9a, 30.3 g (73%) as a cream colored solid. ¹⁹F NMR (376 MHz, DMSO) d −114.17. A solution of 9a (30.2 g, 104 mmol) was suspended in DCM (420 mL). Oxalyl chloride (25.0 mL, 300 mmol) was added followed by DMF (40 mg). After 2 h of stirring, solids were still present and bubbles could still be seen forming. So, the mixture was allowed to stir overnight (16 h). After stirring for 16 h at RT, the solids had dissolved and the mixture became a yellow solution. The solvents were removed in vacuo to afford 33.3 g of the acid chloride as a tan solid. In a separate 500 mL r.b. flask methylimidothiocarbamate sulfate (33.2 g, 177 mmol) was stirred with half-satd. Na₂CO₃ (80 mL) affording a clear solution. Et₂O (60 mL) was added to this solution which was cooled to 10° C. Then, the acid chloride of 9a added slowly as a solution in EtOAc (120 mL), monitoring the temperature with an internal thermometer. A very slight exotherm was observed and the ice bath was removed after the addition was complete. After warming to RT, the mixture was stirred for an 30 min, while monitoring the consumption of the acid chloride using neg. mode ionization and looking for no more 9a present (hydrolysis occurs during LCMS giving the acid). After the reaction was complete, clean product formation was observed and a new peak with M+H=360 with multi-halogen pattern was observed. The mixture was partitioned between water (100 mL) and EtOAc (150 mL) and the aq. layer was extracted with EtOAc (×2). The combined organic extracts were washed with satd. NaHCO₃ (×1), dried over MgSO₄ and concentrated to afford 35 g of 9b (93%) as a tan solid which was used in the next reaction without further purification. ¹H NMR (400 MHz, DMSO-d6) δ 9.03 (br s, 1H), 9.53 (br s, 1H), 2.40 (s, 3H). 9b (14.7 g, 40.7 mmol) was dissolved in DMF (45 mL) and DIEA (14.2 mL, 81.4 mmol) was added. The reaction was heated to 95° C. under N₂ for 3 h at which time LCMS analysis showed clean conversion to the cyclized product (9c) with M+H=282, 284. After cooling to RT, the mixture solution was poured into an aqueous pH 5 buffer and 100 g ice and the resulting solution was adjusted to pH 3 using 6 N HCl. After addition to the cold aqueous solution, a pale-yellow solid precipitated from solution. This precipitate was collected in a Buchner funnel and washed with water (×3) to afford, after drying, 9.6 g of 9c (84%). ¹⁹F NMR (376 MHz, DMSO) δ 135.4. To a flask containing 9c (5.6 g, 20 mmol) was added DIEA (7.1 mL, 28.6 mmol) and the suspension was cooled to 0° C. under N₂. POCl₃ (30 mL, 320 mmol) was added in one portion and the ice bath was removed. The mixture was then heated to 90° C. for 4 h. LCMS analysis (sample dissolved in MeOH) showed two mono-methanol adducts with M+H=294 with Cl₂ isotope pattern. The POCl₃ was removed in vacuo chasing the excess POCl₃ with a mixture of toluene and DCM (×2). After removing all the volatiles, the resulting orange solid was dry loaded on an 80 g ISCO silica column and purified using a gradient of 0-100% EtOAc in heptane, maintaining 100% EtOAc for 7 column volumes as the product bleeds off the column slowly. Concentration of the fractions afforded 5.7 g (95%) of Preparation 9 as an orange solid. ¹³C NMR (101 MHz, DMSO-d6) δ ppm 165.5, 157.2, 148.6, 146.7, 146.0, 143.2, 137.4, 137.3, 114.8, 12.9; ¹⁹F NMR (376 MHz, DMSO) d −135.5.

### Preparation 10: (8aS)-5-chloro-4-fluoro-2-(methylsulfanyl)-8,8a,9,10,11,12-hexahydro-7-oxa-1,3,6,12a-tetraazabenzo[4,5]cyclohepta[1,2,3-de]naphthalene

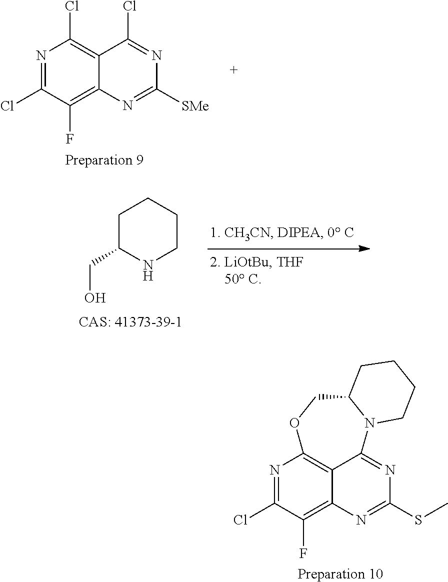

Preparation 9 (1.25 g, 3.70 mmol) was suspended in CH₃CN (24 mL) and DIEA (0.668 mL, 3.83 mmol) was added. The mixture was cooled to 0° C. and (S)-piperidin-2-yl methanol (421 mg, 3.65 mmol) was added as a solution in THE (18 mL). After 8 minutes, the first nitrogen-carbon bond was formed as observed by LCMS. LiOtBu (877 mg, 11.0 mmol) was added as a solution in THE (22 mL) and the mixture was warmed to 50° C. After 4 h at 50° C., LCMS analysis showed conversion to Preparation 9. The reaction mixture was then diluted with 200 mL water and the product was extracted with DCM (50 mL×4). The combined organic extract was dried over Na2SO4, filtered, and evaporated to afford Preparation 10 as a crude solid. Purification was accomplished via flash chromatography eluting with a gradient of 0-10% MeOH in DCM to afford 1.13 g of Preparation 10 (91%). ¹H NMR (CHLOROFORM-d, 400 MHz) d 4.8-4.9 (m, 1H), 4.4-4.5 (m, 2H), 3.7-3.8 (m, 1H), 2.97 (dt, 1H, J=2.5, 12.8 Hz), 2.7-2.7 (m, 1H), 2.6-2.7 (m, 2H), 2.0-2.1 (m, 1H), 1.7-1.8 (m, 3H), 1.5-1.7 (m, 2H), MS: 341.1 [M+H]⁺.

### Preparation 11: (8aS)-4-fluoro-5-[7-fluoro-3-(methoxymethoxy)-8-{[tri(propan-2-yl)silyl]ethynyl}naphthalen-1-yl]-2-(methylsulfanyl)-8,8a,9,10,11,12-hexahydro-7-oxa-1,3,6,12a-tetraazabenzo[4,5]cyclohepta[1,2,3-de]naphthalene

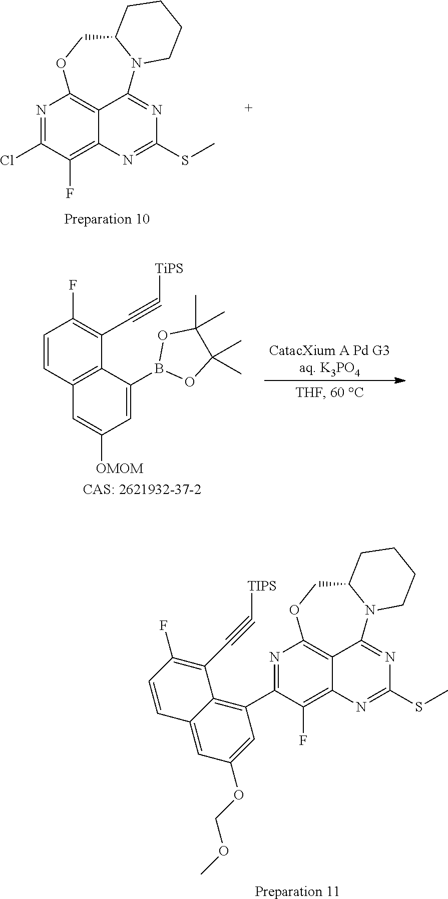

Preparation 10 (1.13 g, 3.32 mmol) and CAS: 2621932-37-2 were dissolved in THE (33 mL). Aqueous K3PO4 (7.3 mL of 1.5 M, 11 mmol) was added and the mixture was purged with N₂ for 5 min. cataCXiumA Pd G3 (241 mg, 0.332 mmol) was added and the and the mixture was purged with N₂ for another 5 min. The reaction was heated at 60° C. for 16 h. LCMS analysis of the mixture showed that CAS: 2621932-37-2 was consumed and some of Preparation 10 remained. The mixture was cooled to RT and diluted with water (40 mL). The mixture was extracted into EtOAc (20 mL×3). The combined organic extract was dried over Na₂SO₄, filtered, and concentrated. The residue was purified by flash chromatography eluting with a gradient of 0-55% EtOAc in heptane. Preparation 11 elutes at 55% EtOAc to afford 1.09 g (48%) as a solid. ¹H NMR (400 MHz, DMSO) δ 8.12-8.03 (m, 1H), 7.72 (t, J=2.1 Hz, 1H), 7.55 (td, J=8.9, 2.6 Hz, 1H), 7.36 (dd, J=6.0, 2.6 Hz, 1H), 5.36 (s, 2H), 5.09 (dd, J=88.0, 13.1 Hz, 1H), 4.54-4.31 (m, 2H), 4.04-3.79 (m, 1H), 3.43 (s, 3H), 3.01 (t, J=12.8 Hz, 1H), 2.52 (d, J=3.7 Hz, 3H), 1.85 (ddd, J=36.1, 23.2, 11.5 Hz, 4H), 1.72-1.47 (m, 1H), 1.45-1.33 (m, 1H), 0.86 (dd, J=7.5, 3.9 Hz, 18H), 0.57 (dq, J=15.0, 7.4 Hz, 3H), ¹⁹F NMR (377 MHz, DMSO) δ−107.27, −143.30, MS: 691.3 [M+H]⁺.

### Preparation 12: (8aS)-4-fluoro-5-[7-fluoro-3-(methoxymethoxy)-8-{[tri(propan-2-yl)silyl]ethynyl}naphthalen-1-yl]-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene

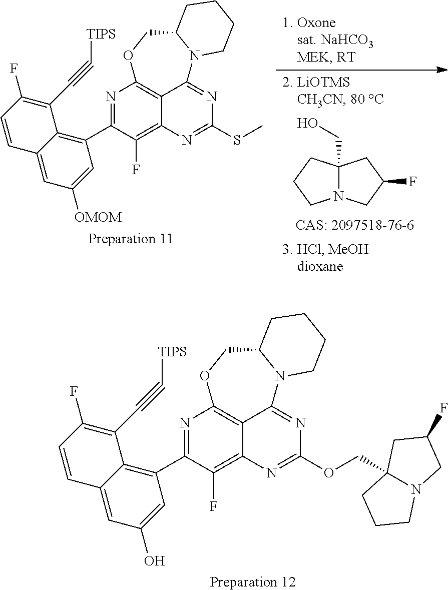

Preparation 11 (944 mg, 1.37 mmol) was dissolved in methylethyl ketone (47 mL) and satd. NaHCO₃ (24 mL) was added followed by Oxone (1.89 g, 3.07 mmol). The reaction was allowed to stir for 40 min. at which time a mixture of sulfone and sulfoxide was observed. The excess Oxone was quenched with satd. Na₂SO₃ and the product was extracted into EtOAc (15 mL×3). The organic extract was washed with brine (×1), dried over Na₂SO₄, filtered, and evaporated to afford 966 mg (100%) of the sulfone/sulfoxide mixture as a solid. MS: 707, 723 [M+H]⁺. This solid was dissolved in CH3CN (10 mL) and ((2R,7AS)-2-fluorohexahydro-1H-pyrrolizin-7A-yl)methanol (288 mg, 1.81 mmol) was added followed by LiOTMS (435 mg, 4.52 mmol). The reaction was heated and stirred at 80° C. for 20 min. LCMS analysis showed the consumption of both sulfone and sulfoxide starting materials. The mixture was cooled to RT and diluted with MeOH (5 mL). HCl (4.8 mL of 4 M in dioxane, 19 mmol) was added. The mixture was stirred at RT for 90 min. at which time complete MOM deprotection was observed. The mixture was evaporated to dryness and EtOAc (80 mL) was added. The EtOAc layer was washed with satd. aq. NaHCO₃ (×3) and the organic layer was collected, dried over Na₂SO₄, filtered, and evaporated onto celite. Purified The Celite supported product was dry loaded onto a 40 g Gold ISCO column and purified by flash chromatography eluting with a gradient of 0-100% EtOAc in heptane followed by 50% MeOH in DCM. Both the desired product and byproduct came out during the 50% MeOH in DCM gradient and the fractions were concentrated and purified a second time using a 24 g Gold ISCO column and eluting with a gradient of 0-20% MeOH in DCM. The pure fractions were pooled and concentrated to afford 910 mg (80%) of Preparation 12 as a solid. ¹H NMR (400 MHz, DMSO) δ 10.07 (d, J=3.2 Hz, 1H), 8.00-7.92 (m, 1H), 7.46 (td, J=9.0, 1.9 Hz, 1H), 7.36 (s, 1H), 7.19 (dd, J=16.8, 2.5 Hz, 1H), 5.29 (d, J=54.1 Hz, 1H), 5.07 (dd, J=81.2, 12.8 Hz, 1H), 4.51-4.31 (m, 2H), 4.13 (dd, J=16.6, 10.4 Hz, 1H), 4.08-3.97 (m, 2H), 3.89 (dd, J=43.2, 9.7 Hz, 1H), 3.17 (d, J=4.7 Hz, 4H), 3.12 (d, J=7.5 Hz, 1H), 3.07-2.79 (m, 3H), 2.18-1.97 (m, 3H), 1.89-1.77 (m, 5H), 0.85 (td, J=6.8, 6.3, 1.8 Hz, 18H), 0.57 (dq, J=14.8, 7.3 Hz, 3H), ¹⁹F NMR (377 MHz, DMSO) δ−109.05, −143.64, −172.29, MS: 758.4 [M+H]⁺.

### Preparation 13: {[2,3-difluoro-8-(4,4,5,5-tetramethyl-1,3,2-dioxaborolan-2-yl)-6-{[2-(trimethylsilyl)ethoxy]methoxy}naphthalen-1-yl]ethynyl}tri(propan-2-yl)silane

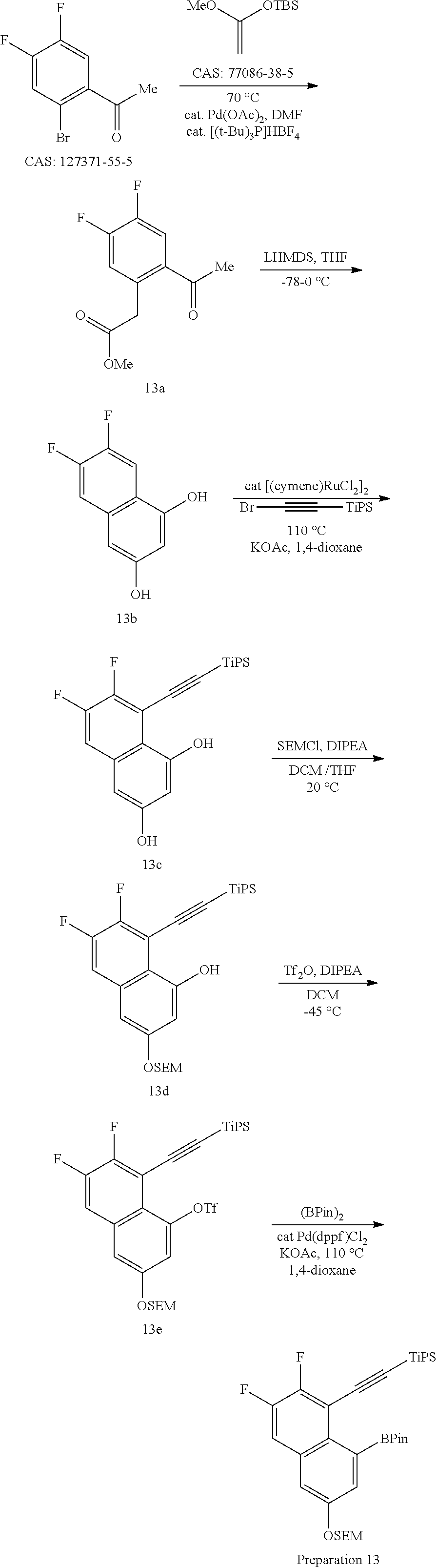

To a solution of CAS 127371-55-5 (10.0 g, 42.5 mmol) and CAS 77086-38-5 (32.1 g, 170 mmol) in dry DMF (100 mL) was added [(t-Bu)₃P]HBF₄ (4.94 g, 17.0 mmol), LiF (15.5 g, 596 mmol) and Pd(OAc)₂ (1.91 g, 8.51 mmol) successively under N₂. Then the mixture was stirred at 70° C. for 1 h at which time LCMS analysis showed that the starting methyl ketone had been consumed. After cooling to rt, the mixture was filtered through a pad of Celite, and the filtrate was diluted with water (500 mL). The product was extracted using EtOAc (4×250 mL) and the combined organic phase was washed with brine (3×200 mL), dried over Na₂SO₄ and concentrated. The residue was purified by flash chromatography using a gradient of 0-35% EtOAc in petroleum ether to afford 8.08 g of 13a (83%) as a white solid. ¹H NMR (400 MHz, DMSO) δ δ 8.10 (dd, J=11.5, 8.3 Hz, 1H), 7.52 (dd, J=11.6, 8.0 Hz, 1H), 3.88 (s, 2H), 3.58 (s, 3H), 2.54 (s, 3H), MS: 229 [M+H]⁺. 13a (6.98 mg, 30.6 mmol) was then dissolved in dry THE (306 mL) and cooled to −78° C. under N₂. LHMDS (55 mL of 1.0 M in THF, 55 mmol) was added and the mixture was stirred at 0° C. for 2 h. Analysis by LCMS showed that the starting ketoester had been consumed and the mixture was quenched with 1 M HCl (55 mL) and diluted with water (200 mL). The aqueous layer was extracted with EtOAc (4×200 mL) and the combined organic extract was washed with brine and dried over Na₂SO₄. After concentration of the extract, crude 13b (6.42 g) was obtained as a red solid. ¹H NMR (400 MHz, MeOD) δ 7.81-7.71 (m, 1H), 7.39-7.29 (m, 1H), 6.59 (d, J=2.1 Hz, 1H), 6.47 (d, J=2.0 Hz, 1H), MS: 197 [M+H]⁺. This crude material was taken to the next step without further purification. To a solution of 13b (6.42 g, 32.7 mmol), and CAS 111409-79-1 (10.3 g, 39.3 mmol) in dry 1,4-dioxane (113 mL) was added dichloro(p-cymene)ruthenium(II) dimer (2.01 g, 3.27 mmol) and KOAc (6.43 g, 65.5 mmol) under N₂. Then the mixture was stirred at 110° C. for 16 h. Analysis by LCMS showed that the starting material was consumed. After cooling to rt, the mixture was filtered through a pad of Celite and the filtrate was diluted with water (200 mL). The organic product was extracted into EtOAc (4×200 mL) and the combined organic extract was washed with brine (3×20 mL), dried over Na₂SO₄ and concentrated. The crude product was purified by flash chromatography using a gradient of 0-30% EtOAc in petroleum ether to afford 7.19 g of 13c (58%) as a brown solid. MS: 377 [M+H]⁺. To a mixture of 13c (7.19 g, 19.1 mmol) and DIPEA (3.7 g, 28.7 mmol) in DCM (100 mL) and THE (10 mL) was added SEMCl (3.19 g, 19.1 mmol) and the reaction was stirred at 20° C. for 24 h. Analysis by LCMS showed that the starting material was consumed, and the desired product was detected. The mixture was concentrated then the residue was purified by silica gel column chromatography using a gradient of 0-5% EtOAc in petroleum ether) to afford 7.64 g of 13d (79%) as yellow oil. ¹H NMR (400 MHz, CDCl₃) δ 8.99 (s, 1H), 7.40 (dd, J=10.8, 7.9 Hz, 1H), 6.89 (d, J=2.4 Hz, 1H), 6.75 (d, J=2.3 Hz, 1H), 5.28 (d, J=3.3 Hz, 2H), 3.80-3.75 (m, 2H), 1.27 (d, J=7.9 Hz, 2H), 1.18 (d, J=5.4 Hz, 18H), 1.00-0.94 (m, 3H), 0.00 (s, 9H). To a mixture of 13d (7.64 g, 15.1 mmol) in DCM (84 mL) was added DIPEA (3.9 g, 30.2 mmol) and the solution was cooled to −30° C. before adding Tf₂O (6.38 g, 22.6 mmol) dropwise. The resulting mixture was stirred at −30° C. for 1 h at which time TLC analysis showed a new spot. The resulting mixture was then poured into water (200 mL) and extracted with DCM (2×100 mL). The combined organic extract was washed with brine (50 mL) and dried over anhydrous Na₂SO₄. The mixture was concentrated, and the residue was purified by flash chromatography eluting with a gradient of 0-2% EtOAc in petroleum ether to afford 5.69 g of 13e (59%) as a yellow solid. ¹H NMR (400 MHz, CDCl₃) δ 7.46 (dd, J=10.1, 7.7 Hz, 1H), 7.35 (d, J=2.3 Hz, 1H), 7.30 (d, J=2.2 Hz, 1H), 5.31 (s, 2H), 3.78 (dd, J=9.7, 4.3 Hz, 2H), 1.24 (dd, J=5.0, 3.3 Hz, 2H), 1.17 (t, J=5.0 Hz, 18H), 1.02-0.90 (m, 3H), 0.00 (s, 9H). To a solution of 13e (5.69 g, 8.90 mmol) and B₂Pin₂ (4.52 g, 17.8 mmol) in 1,4-dioxane (45 mL) was added Pd(dppf)Cl₂ (651 mg, 0.890 mmol) and KOAc (2.62 g, 26.7 mmol) under N₂. Then the reaction was stirred at 110° C. for 16 h. LCMS analysis showed the starting material was consumed and the mixture was filtered through a pad of Celite. The filtrate was concentrated to give a crude which was purified by flash chromatography eluting with a gradient of 0-5% EtOAc in petroleum ether. Fractions containing the desired product were concentrated to afford 2.24 g of Preparation 13 (41%) as yellow solid. ¹H NMR (400 MHz, CDCl₃) δ 7.45-7.44 (m, 1H), 7.43-7.38 (m, 1H), 7.31 (d, J=2.6 Hz, 1H), 5.30 (s, 2H), 3.80-3.74 (m, 2H), 1.42 (s, 12H), 1.17 (d, J=3.3 Hz, 18H), 0.98-0.94 (m, 2H), 0.87 (dd, J=6.0, 2.7 Hz, 2H), 0.00 (s, 9H), MS: 617 [M+H]⁺. Using General Method D, boronic ester Preparation 13 was coupled to Preparation 10 to afford Example 32 in 5 steps.

### EXAMPLES

In order that this invention may be better understood, the following examples are set forth. These examples are for purposes of illustration only and are not to be construed as limiting the scope of the invention in any manner.

### Example 1: {(3S)-1-[7-(8-ethynyl-3-hydroxynaphthalen-1-yl)-8-fluoro-2-{[(2R,7aS)-2-fluorotetrahydro-1H-pyrrolizin-7a(5H)-yl]methoxy}pyrido[4,3-d]pyrimidin-4-yl]piperidin-3-yl}acetonitrile

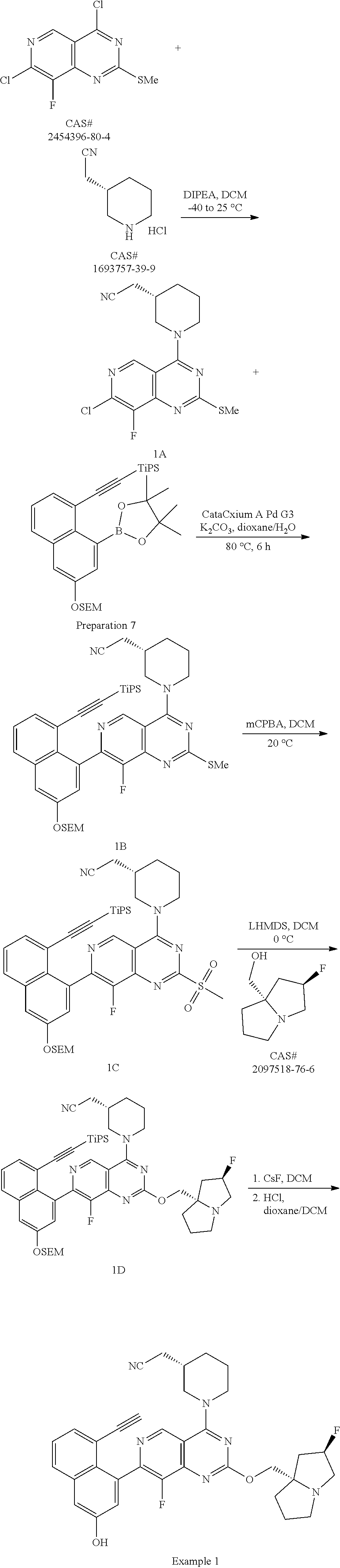

Example 1 was prepared according to the above scheme, which is representative of General Method A.

To a solution of CAS #2454396-80-4 (215 mg, 0.814 mmol) and CAS #1693757-39-9 (131 mg, 0.819 mmol) in DCM (10 mL) was added DIPEA (316 mg, 2.44 mmol) at −40° C. under argon. The mixture was stirred at 25° C. for 2 h. The mixture was warmed to rt and diluted with water (30 mL). The aqueous layer was extracted with DCM (3×30 mL). The combined organic layer was washed with brine (30 mL) and dried over Na₂SO₄, filtered and concentrated. The residue was purified by flash chromatography using a gradient of 0-60% EtOAc in petroleum ether to afford 1A (160 mg, 56%) as yellow solid. ¹H NMR (400 MHz, DMSO-d₆) δ 8.88 (s, 1H), 4.48 (d, J=11.6 Hz, 1H), 4.38 (d, J=13.4 Hz, 1H), 3.38 (t, J=10.9 Hz, 1H), 3.27-3.20 (m, 1H), 2.63 (dd, J=6.7, 4.0 Hz, 2H), 2.59-2.52 (m, 3H), 2.14-2.05 (m, 1H), 1.94 (d, J=9.8 Hz, 1H), 1.87-1.79 (m, 1H), 1.71-1.62 (m, 1H), 1.49-1.40 (m, 1H), MS: 352 [M+H]⁺. To a solution of 1A (160 mg, 0.455 mmol) and Preparation 7 (343 mg, 0.591 mmol) in dioxane (10 mL) and H₂O (1 mL) was added CataCXium A Pd G3 (33.1 mg, 0.0455 mmol) and K₂CO₃ (189 mg, 1.36 mmol) under N₂ in a tube. The reaction was stirred at 80° C. for 6 h. The mixture was concentrated under reduced pressure and the residue was purified by flash chromatography using a gradient of 0-50% EtOAc in petroleum ether to afford 1B (207 mg, 59%) as yellow solid. ¹H NMR (400 MHz, DMSO) δ 9.10 (d, J=4.6 Hz, 1H), 8.04 (d, J=8.3 Hz, 1H), 7.72 (s, 1H), 7.69-7.63 (m, 1H), 7.58 (t, J=7.7 Hz, 1H), 7.33 (d, J=2.3 Hz, 1H), 5.46 (s, 2H), 4.57 (d, J=11.8 Hz, 1H), 4.48 (s, 1H), 3.88-3.76 (m, 2H), 3.20 (d, J=10.8 Hz, 1H), 2.71 (d, J=6.8 Hz, 2H), 2.60 (d, J=2.2 Hz, 3H), 2.10-1.92 (m, 3H), 1.51 (d, J=11.7 Hz, 1H), 1.28 (s, 1H), 1.00-0.94 (m, 2H), 0.93-0.80 (m, 18H), 0.53 (dq, J=14.4, 7.1 Hz, 3H), 0.05 (s, 1H), −0.00 (s, 9H), MS: 770 [M+H]⁺. To a solution of 1B (207 mg, 0.269 mmol) in DCM (10 mL) was added mCBPA (69.6 mg, 0.403 mmol). The reaction was stirred at 20° C. for 1 h. The reaction mixture was diluted with satd. aq. Na₂SO₃ (20 mL) and extracted with DCM (3×20 mL). The combined organic layer was washed with saturated NaHCO₃ (100 mL), brine (100 mL) and dried over anhydrous Na₂SO₄. After concentration, crude 1C (211 mg, 99%) was obtained as a yellow solid. ¹H NMR (400 MHz, DMSO) δ 9.31 (s, 1H), 8.07 (d, J=8.2 Hz, 1H), 7.76 (s, 1H), 7.67 (s, 1H), 7.60 (t, J=8.0 Hz, 1H), 7.36 (s, 1H), 5.47 (s, 2H), 4.67 (s, 2H), 3.81 (t, J=7.8 Hz, 2H), 2.98 (dd, J=11.1, 6.6 Hz, 3H), 2.76 (s, 2H), 2.04 (d, J=7.8 Hz, 2H), 1.28 (s, 3H), 0.96 (t, J=8.2 Hz, 2H), 0.86 (dd, J=12.3, 5.1 Hz, 18H), 0.54 (dt, J=14.9, 7.4 Hz, 3H), 0.05 (s, 3H), −0.00 (s, 9H), MS: 786 [M+H]⁺. To a solution of 1C (211 mg, 0.263 mmol) and CAS #2097518-76-6 (50.3 mg, 0.316 mmol) in dry DCM (10 mL) was dropwise added LHMDS (0.289 mL of 1 M in THF, 0.289 mmol) under N₂ at 0° C. The reaction was stirred at 0° C. for 1 h. The reaction mixture was diluted with H₂O (15 mL) and extracted with DCM (3×10 mL). The combined organic layer was washed with brine (100 mL) and dried over anhydrous Na₂SO₄. After concentrating under reduced pressure, the resulting residue was purified by flash chromatography eluting with a gradient of 0-10% MeOH in DCM to afford 1D (161 mg, 69%) as a yellow solid. ¹H NMR (400 MHz, DMSO) δ 9.09 (d, J=3.8 Hz, 1H), 8.04 (d, J=8.2 Hz, 1H), 7.72 (d, J=2.5 Hz, 1H), 7.69-7.63 (m, 1H), 7.62-7.54 (m, 1H), 7.32 (d, J=1.9 Hz, 1H), 5.46 (s, 2H), 5.27 (s, 1H), 4.48 (dd, J=42.3, 11.5 Hz, 3H), 4.18 (dd, J=23.5, 10.5 Hz, 1H), 4.09-3.97 (m, 1H), 3.90-3.67 (m, 2H), 3.19 (dd, J=25.0, 11.6 Hz, 3H), 3.07 (s, 1H), 2.89 (s, 1H), 2.77-2.67 (m, 2H), 2.11-2.01 (m, 2H), 1.96 (d, J=13.2 Hz, 2H), 1.80 (d, J=11.7 Hz, 2H), 1.52 (d, J=13.4 Hz, 2H), 0.97 (dd, J=18.9, 10.0 Hz, 3H), 0.91-0.82 (m, 18H), 0.54 (tt, J=15.0, 6.0 Hz, 3H), 0.11-0.02 (m, 3H), 0.02-−0.04 (m, 9H), MS: 881 [M+H]⁺. To a solution of 1D (160 mg, 0.182 mmol) in DMF (5 mL) was added CsF (276 mg, 1.82 mmol). Then the reaction was stirred at 25° C. for 1 h. The reaction mixture was diluted with H₂O (30 mL) and extracted with EtOAc (3×20 mL). The combined organic layer was washed with brine (100 mL) and dried over anhydrous Na₂SO₄. After concentrating under reduced pressure, the crude terminal alkyne (132 mg, 99%) was obtained as yellow solid and was used for next step without further purification. MS: 725 [M+H]⁺. To a solution of the crude terminal alkyne (120 mg, 0.166 mmol) in DCM (10 mL) was added HCl (0.20 mL of 4 M in dioxane, 0.80 mmol). The reaction was stirred at 25° C. for 0.5 h. The reaction was concentrated and the crude product was purified by prep-HPLC (column: Xbridge 5m C18 150×19 mm; mobile phase: CH₃CN-Water (0.1% formic acid); gradient: 23%-100%; flow rate: 20 mL/min) to give {(3S)-1-[7-(8-ethynyl-3-hydroxynaphthalen-1-yl)-8-fluoro-2-{[(2R,7aS)-2-fluorotetrahydro-1H-pyrrolizin-7a(5H)-yl]methoxy}pyrido[4,3-d]pyrimidin-4-yl]piperidin-3-yl}acetonitrile, Example 1 (25.2 mg, 24%) as a yellow solid. ¹H NMR (400 MHz, DMSO-d₆) δ 9.00 (d, J=10.0 Hz, 1H), 7.88 (d, J=8.0 Hz, 1H), 7.50-7.39 (m, 2H), 7.34 (d, J=2.4 Hz, 1H), 7.14 (d, J=2.3 Hz, 1H), 5.48-5.15 (m, 1H), 4.45 (dd, J=25.6, 13.0 Hz, 3H), 4.25-4.10 (m, 1H), 4.10-4.00 (m, 1H), 3.62 (d, J=3.7 Hz, 1H), 3.20-2.97 (m, 5H), 2.83 (d, J=6.6 Hz, 1H), 2.66 (d, J=7.5 Hz, 2H), 2.13 (d, J=4.7 Hz, 2H), 2.05 (s, 1H), 2.01 (s, 2H), 1.92-1.71 (m, 4H), 1.47 (s, 1H), MS: 595 [M+H]⁺.

Examples 2-9 reported in Table 1 were prepared according to Scheme I/General Method A with non-critical modifications that one skilled in the art would appreciate.

### Example 10: (1R,5R,6R)-3-[7-(8-ethynyl-7-fluoro-3-hydroxynaphthalen-1-yl)-8-fluoro-2-{[(2R,7aS)-2-fluorotetrahydro-1H-pyrrolizin-7a(5H)-yl]methoxy}pyrido[4,3-d]pyrimidin-4-yl]-3-azabicyclo[3.2.1]octan-6-ol

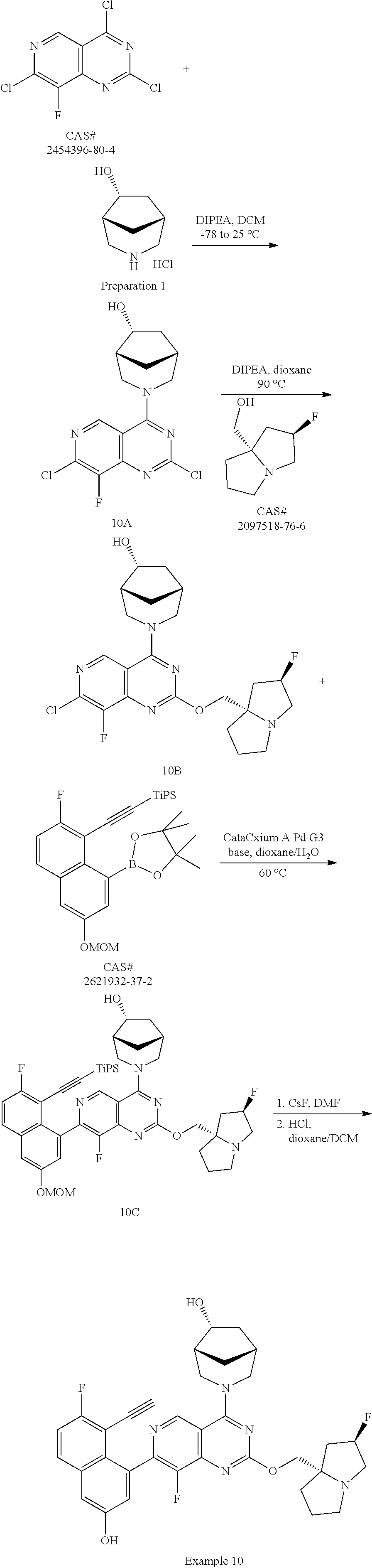

Example 10 was prepared according to the above scheme, which is representative of General Method B.

A solution of CAS #2454396-80-4 (913 mg, 3.61) and Preparation 1 (591 mg, 3.61 mmol) was dissolved in DCM (72 mL). The solution was cooled to −78° C. and DIPEA (1.57 mL, 9.04 mmol) was added. The reaction was stirred at −78° C. for 1 h and the cold bath was removed. The reaction was allowed to warm to rt over 1 h. The solvent was removed and the residue purified via flash chromatography eluting with a gradient of 0-100% EtOAc in heptane. After concentrating the pure fractions, 10A (1.09 g, 88%) was obtained as a yellow solid. ¹H NMR (400 MHz, DMSO-d₆) δ ppm 1.17-1.24 (m, 1H) 1.69 (s, 1H) 1.79 (s, 1H) 2.05-2.15 (m, 1H) 2.16-2.22 (m, 1H) 2.33-2.38 (m, 1H) 3.45 (br d, J=12.6 Hz, 1H) 3.74 (br d, J=12.5 Hz, 1H) 4.15-4.22 (m, 1H) 4.46 (br s, 1H) 4.58 (br d, J=13.3 Hz, 1H) 4.66-4.73 (m, 1H) 9.21 (s, 1H), MS: 343, 345 [M+H]⁺. A solution of 10A (1.22 g, 3.55 mmol) in dioxane (20 mL) was treated with DIPEA (1.24 mL, 7.11 mmol) and ((2R,7AS)-2-fluorohexahydro-1H-pyrrolizin-7A-yl)methanol (1.13 g, 7.11 mmol) and the mixture was heated to 90° C. for 48 h. The reaction was diluted with EtOAc (100 mL) and washed with water and brine. The organic extract was dried over sodium sulfate and concentrated to give an orange oil. The crude orange oil was purified via SFC to afford 10B (890 mg, 54%). ¹H NMR (400 MHz, DMSO-d₆) δ ppm 9.08 (s, 1H) 5.16-5.41 (m, 1H) 4.62-4.75 (m, 2H) 4.47 (br d, J=12.1 Hz, 1H) 4.08-4.19 (m, 2H) 4.00 (d, J=10.3 Hz, 1H) 3.63-3.74 (m, 1H) 3.33 (s, 1H) 2.98-3.14 (m, 3H) 2.73-2.91 (m, 1H) 2.31 (br s, 1H) 1.93-2.20 (m, 5H) 1.72-1.91 (m, 4H) 1.57-1.69 (m, 1H) 1.11-1.26 (m, 1H), MS 466 [M+H]⁺. To a mixture of 10B (100 mg, 0.215 mmol) and CAS #2621932-37-2 (132 mg, 0.258 mmol) in THE (2.5 mL) was added K₃PO₄ (150 mg, 0.708 mmol) and water (0.25 mL). Nitrogen was bubbled through the solution for 10 min, and CataCXium A Pd G3 (15.6 mg, 0.0215 mmol) was added. The vial was sealed and heated to 60° C. for 2 h. LCMS shows a clean reaction and about 50% conversion to the Suzuki product. Heating was continued at 60° C. for 18 h longer. The mixture was concentrated under vacuum and the resulting residue purified by flash chromatography eluting with a gradient of 0-20% iPrOH in DCM. After concentrating the pure fractions, 10C (130 mg, 74%) was obtained as a yellow powder. ¹H NMR (400 MHz, DMSO-d₆) δ=9.62, 9.26 (2s, 1H, minor and major rotamer respectively), 8.10 (dd, J=5.9, 9.3 Hz, 1H), 7.73 (d, J=2.4 Hz, 1H), 7.56 (t, J=8.9 Hz, 1H), 7.39-7.31 (m, 1H), 5.40-5.34 (m, 2H), 5.23 (br s, 1H), 4.97-4.84 (m, 1H), 4.68, 4.50 (2d, J=3.4 Hz, br d, J=11.5 Hz, 1H, minor and major rotamer respectively), 4.32-4.05 (m, 3H), 4.01-3.91 (m, 1H), 3.82-3.73 and 3.60-3.53 (2m, 1H, major and minor rotamer respectively), 3.44 (s, 2H), 3.16-2.99 (m, 2H), 2.91-2.79 (m, 1H), 2.52-2.35 (m, 4H buried under DMSO peak), 2.40-2.30 (m, 1H), 2.26-1.96 (m, 4H), 1.91-1.61 (m, 4H), 1.36-1.22 (m, 1H), 0.87-0.78 (m, 18H), 0.56-0.42 (m, 3H); MS: 816 [M+H]⁺. To a solution of 10C (126 mg, 0.154 mmol) in CH₃CN (5 mL) was added CsF (235 mg, 1.55 mmol). The reaction was stirred at rt for 4 h. The mixture was neutralized with acetic acid (22 mL, 0.386 mmol) and concentrated under vacuum. The residue was taken up in EtOAc and the salts were removed by filtration. The filtrate was concentrated to give the terminal alkyne as a peach-colored powder (102 mg) that was taken into the next step without further purification. A suspension of the terminal alkyne from the previous step (102 mg, 0.154 mmol) in CH₃CN (2 mL) was cooled in an ice bath. 4N HCl (2 mL of 4 N in dioxane, 8.0 mmol) was added. The r×n was stirred at 0° C. for 1 h. The solvent was evaporated to give an orange solid. This residue was dissolved in MeOH (1 mL) and purified by prep-HPLC using acetic acid as additive to the CH₃CN/water eluent. The pure fractions were combined and concentrated to 15 mL of water. The aqueous solution was frozen at −78° C. and lyophilized overnight to give (1R,5R,6R)-3-[7-(8-ethynyl-7-fluoro-3-hydroxynaphthalen-1-yl)-8-fluoro-2-{[(2R,7aS)-2-fluorotetrahydro-1H-pyrrolizin-7a(5H)-yl]methoxy}pyrido[4,3-d]pyrimidin-4-yl]-3-azabicyclo[3.2.1]octan-6-ol, Example 10 (104 mg, 64%) as a pale orange powder. ¹H NMR (400 MHz, METHANOL-d4) δ=9.28-9.04 (m, 1H), 7.90-7.81 (m, 1H), 7.35 (d, J=2.4 Hz, 1H), 7.34-7.28 (m, 1H), 7.27-7.19 (m, 1H), 5.49-5.30 (m, 1H), 5.21 (br d, J=11.9 Hz, 1H), 4.67 (br d, J=12.1 Hz, 1H), 4.50-4.42 (m, 1H), 4.38-4.26 (m, 2H), 3.98-3.72 (m, 1H), 3.58-3.34 (m, 5H), 3.21-3.11 (m, 1H), 2.51-2.05 (m, 9H), 2.03-1.89 (m, 5H), 1.86-1.75 (m, 1H), 1.46-1.36 (m, 1H), HRMS: 616.25385 [M+H]⁺.

Examples 11-17 reported in Table 1 were prepared according to General Method B/Example 10 with non-critical modifications that one skilled in the art would appreciate.

### Examples 18: (1R*,5R*,6R*)-3-[7-(8-ethynyl-3-hydroxynaphthalen-1-yl)-8-fluoro-2-{[(2R,7aS)-2-fluorotetrahydro-1H-pyrrolizin-7a(5H)-yl]methoxy}pyrido[4,3-d]pyrimidin-4-yl]-8-oxa-3-azabicyclo[3.2.1]octan-6-ol

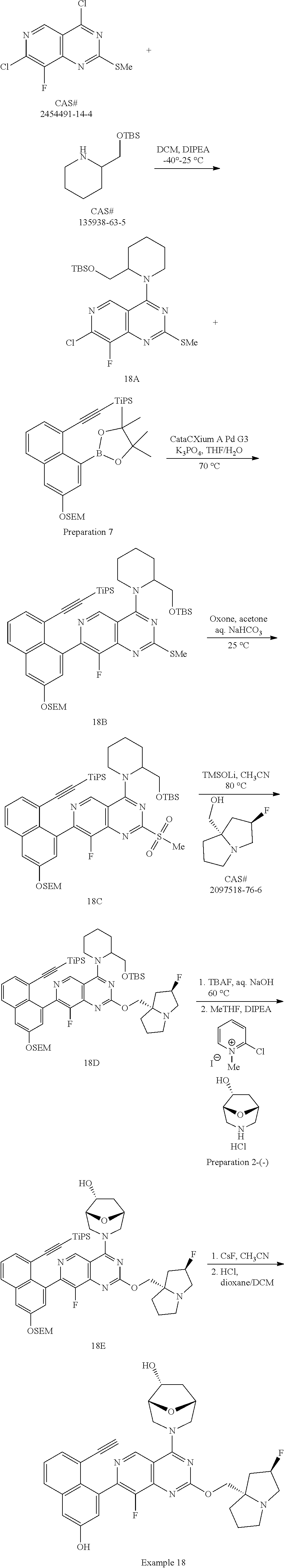

Example 18 was prepared according to the above scheme, which is representative of General Method C.

Compound CAS #2454491-14-4 (3.66 g, 13.8 mmol) was combined with CAS #135938-63-5 (3.18 g, 13.9 mmol) in DCM (69 mL). The resulting solution was cooled to −40° C. and DIPEA (7.0 mL, 42 mmol) was added. The mixture was allowed to gradually warm from −40° C. to rt over 3.5 h. The mixture was partitioned between water and DCM and the DCM layer was washed with water (3×). The organic layer was dried over sodium sulfate and resubjected to the reaction conditions adding fresh CAS #135938-63-5 (1.6 g, 6.9 mmol) as well as fresh DIPEA (7.0 mL, 42 mmol). After stirring for 1 h, a third portion of CAS #135938-63-5 (1.6 g, 6.9 mmol) was added and the reaction was allowed to stir at rt for 18 h. The reaction mixture was partitioned between water and DCM and the DCM layer was washed with water (3×). The organic layer was dried over sodium sulfate and evaporated. Purification via flash chromatography using a gradient of 0-100% EtOAc in heptane afforded 18A (4.26 g, 67%) as a glass. ¹H NMR (400 MHz, CDCl₃) δ 8.94 (s, 1H), 4.84 (s, 1H), 4.46 (d, J=13.5 Hz, 1H), 4.12 (dd, J=10.5, 8.6 Hz, 1H), 3.76 (dd, J=10.5, 5.7 Hz, 1H), 3.34 (t, J=12.3 Hz, 1H), 2.60 (s, 3H), 1.88-1.64 (m, 9H), 0.79 (s, 8H), 0.02 (d, J=2.3 Hz, 6H), ¹⁹F NMR (376 MHz, CDCl₃) δ −134.84, MS: 457.1 [M+H]⁺. 18A (197 mg, 0.430 mmol) was combined with Preparation 7 (500 mg, 0.861 mmol) in THE (4.3 mL) and aqueous K₃PO₄ (1.43 mL of 1.5 M, 2.15 mmol). The mixture was purged with nitrogen for 3 min. CataCXium A Pd G3 (31.3 mg, 0.0430 mmol) was then added and the mixture purged with nitrogen for an additional 3 min. The reaction was heated at 70° C. for 4.5 h and product formation was observed by TLC. The mixture was then evaporated directly onto celite. The celite was packed into an Isco cartridge and the product was purified by flash chromatography eluting with a gradient of 0-100% EtOAc in heptane. The fractions were analyzed using a non-polar/high mass LCMS method which picked up the target mass as a late eluting peak. Concentration of the pure fractions afforded Compound 18B (367 mg, 49%) as a glassy orange solid. ¹H NMR (400 MHz, CDCl₃) δ 9.11 (d, J=20.0 Hz, 1H), 7.81 (dt, J=8.3, 1.5 Hz, 1H), 7.68 (dd, J=7.2, 1.2 Hz, 1H), 7.54 (t, J=3.0 Hz, 1H), 7.42-7.38 (m, 1H), 7.30 (dd, J=4.3, 2.6 Hz, 1H), 5.37 (d, J=7.0 Hz, 1H), 5.35-5.31 (m, 1H), 4.87 (s, 1H), 4.38 (dd, J=38.0, 13.8 Hz, 1H), 4.05-3.96 (m, 1H), 3.84-3.74 (m, 3H), 3.51-3.32 (m, 1H), 2.61 (d, J=1.3 Hz, 3H), 1.27 (q, J=2.2 Hz, 4H), 1.19-1.16 (m, 4H), 1.05 (d, J=1.1 Hz, 3H), 1.00-0.95 (m, 3H), 0.90 (d, J=1.6 Hz, 9H), 0.88 (t, J=1.8 Hz, 9H), 0.77 (s, 6H), 0.58-0.49 (m, 3H), −0.00 (t, J=1.2 Hz, 9H), −0.03 (d, J=2.5 Hz, 3H), MS: 875.4 [M+H]⁺. Compound 18B (367 mg, 0.419 mmol) was dissolved in a mixture of acetone (20 mL) and saturated aqueous sodium bicarbonate (10 mL). Oxone (322 mg, 0.524 mmol) was added and the mixture was stirred at rt for 45 min. The pH was tested with pH paper and was observed to be between 7-8. The reaction was quenched by the addition of saturated aqueous sodium sulfite (20 mL) and the reaction mixture was stirred for 5 minutes. The reaction mixture was extracted using 50/50 heptane:EtOAc (1×40 mL) and the organic extract was washed with brine (1×25 mL) and dried over sodium sulfate. After filtration and concentration, 18C (341 mg, 90%) was obtained. Note: both sulfoxide and sulfone masses were observed under a single broad peak. MS: sulfoxide: 891.4, sulfone: 907.4 [M+H]⁺. This material was taken to the next step without further purification. Compound 18C (341 mg, 0.376 mmol) was combined with CAS #2097518-76-6 (89.7 mg, 0.564 mmol) and lithium trimethylsilanolate (108 mg, 1.13 mmol) in CH₃CN (4.18 mL). The mixture was heated at 80° C. for 30 min. After cooling, solids were removed by filtration. The filtrate was diluted with water (40 mL) and extracted using a solvent mixture of 10% EtOAc in heptane (3×10 mL). The combined organic extract was dried over sodium sulfate, filtered and concentrated. The residue was purified via flash chromatography by dissolving in 5 mL heptane and loading directly onto a silica column. Gradient elution using 0-100% EtOAc in heptane afforded 18D (236 mg, 64%). MS: 986.5 [M+H]⁺. Compound 18D (236 mg, 0.239 mmol) was dissolved in THE (2.39 mL) and aqueous NaOH (1.20 mL of 1 M, 1.20 mmol) was added followed by TBAF (1.20 mL of 1 M in THF, 1.20 mmol) and the mixture was heated to 60° C. with stirring. After 2.3 h, the reaction mixture was cooled to rt, and diluted with EtOAc (30 mL). The organic layer was washed with brine (6×25 mL) to remove the excess TBAF. The organic layer was dried over sodium sulfate, filtered, and evaporated. The residue was purified by flash chromatography eluting with a gradient of 0-100% EtOAc (containing 10%, by volume, 7 N ammonia in methanol) in heptane. The pure fractions were collected to provide (50 mg, 27%) and the column was further flushed with 75% methanol in DCM to elute the remaining target material retained on the column to give another 100 mg of product. The overall yield of the C4 deprotection step was 150 mg, 81%, MS: 775.3 [M+H]⁺. Half of this material was taken directly on to the amine coupling step. The material from the TBAF/NaOH treatment (50 mg, 0.065 mmol) was combined with CMPI (26.4 mg, 0.103 mmol) and DIPEA (57.4 μL, 0.323 mmol) in DCM (1.0 mL). The mixture was stirred at rt for 1 h at rt. Then, Preparation 2-(−) (17.1 mg, 0.103 mmol) was added and the mixture was stirred at rt for 3 h. The reaction mixture was diluted with EtOAc (30 mL) and the organic layer was washed with brine (3×10 mL). The organic layer was dried over sodium sulfate, filtered, and evaporated to afford 18E (52.0 mg, 91%). MS 886.4 [M+H]⁺. 18E (52 mg, 0.059 mmol) was dissolved in CH₃CN (1.0 mL) and CsF (89.1 mg, 0.587 mmol) was added. The reaction mixture was stirred at rt for 6 h to complete the removal of the TiPS group. Then, HCl (0.293 mL of 4.0 M in dioxane, 1.17 mmol) was added. The reaction was allowed to stir for 8 h. The reaction mixture was diluted with EtOAc (30 mL) and the organic layer was washed with a 50/50 solution of brine and aqueous 1 M NaOH (3×20 mL). The organic layer was dried over sodium sulfate, filtered, and evaporated to afford 46 mg of crude Example 18. Purification by reverse phase HPLC afforded (1R*,5R*,6R*)-3-[7-(8-ethynyl-3-hydroxynaphthalen-1-yl)-8-fluoro-2-{[(2R,7aS)-2-fluorotetrahydro-1H-pyrrolizin-7a(5H)-yl]methoxy}pyrido[4,3-d]pyrimidin-4-yl]-8-oxa-3-azabicyclo[3.2.1]octan-6-ol, Example 18, ¹H NMR (400 MHz, DMSO) δ 9.07 and 9.31 (2 s, 1H, major and minor rotamer respectively), 7.87 (dt, J=8.0, 2.3 Hz, 1H), 7.46-7.39 (m, 2H), 7.34 (t, J=2.5 Hz, 1H), 7.17 and 7.11 (2 d, J=2.6 Hz, 1H, major and minor rotamer respectively), 5.26 (d, J=54.3 Hz, 1H), 4.42 (d, J=7.3 Hz, 1H), 4.33-4.19 (m, 2H), 4.16-3.98 (m, 4H), 3.44 (d, J=11.9 Hz, 1H), 3.07 (d, J=9.5 Hz, 2H), 2.99 (s, 1H), 2.81 (t, J=7.9 Hz, 1H), 2.34-2.25 (m, 1H), 2.11-2.02 (m, 2H), 1.77 (d, J=8.2 Hz, 8H), ¹⁹F NMR (376 MHz, DMSO) δ−141.48, −172.18 (only major rotamers reported), MS: 600.2 [M+H]⁺.

Examples 19-22 reported in Table 1 were prepared according to General Method C/Example 18 with non-critical modifications that one skilled in the art would appreciate.

Additional compounds of the invention were prepared by modifications of the methods exemplified herein. Except where otherwise indicated, all compounds having chiral centers were prepared and/or isolated as a single enantiomer having a known relative configuration. Compounds marked “absolute stereochemistry unknown” were typically prepared from racemic intermediates and resolved into single enantiomers by an appropriate chiral preparative SEC method before characterization and testing.

Examples 1-22 and their corresponding characterization data are all presented in Table 1 below.

TABLE 1
Examples 1-22

| Ex. No. | | LCMS | ¹H NMR (ppm); ¹⁹F NMR (ppm); |
|---|---|---|---|
| (Gen. Method) | Structure/IUPAC name | [M + H]⁺ | optical rotation; stereochem. notes |
| 1 (A) | 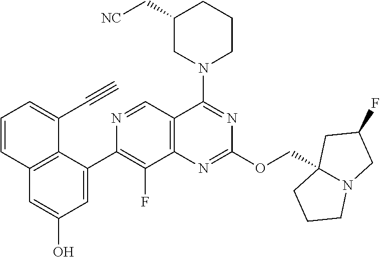 [SMILES: C#Cc1cccc2cc(O)cc(-c3ncc4c(N5CCC[C@@H](CC#N)C5)nc(OC[C@@]56CCCN5C[C@H](F)C6)nc4c3F)c12] {(3S)-1-[7-(8-ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]piperidin-3- yl}acetonitrile | 595.2 | ¹H NMR (400 MHz, DMSO-d₆) δ 9.01 and 8.98 (2 s, 1H, two rotamers respectively), 7.89-7.87 (m, 1H), 7.47-7.39 (m, 2H), 7.34-7.33 (m, 1H), 7.14-7.13 (m, 1H), 5.35-5.21 (m, 1H), 4.53-4.30 (m, 3H), 4.18- 4.09 (m, 1H), 4.07-4.00 (m, 1H), 3.63-3.62 (m, 1H), 3.19-2.98 (m, 5H), 2.87-2.80 (m, 1H), 2.72-2.63 (m, 2H), 2.21-2.10 (m, 2H), 2.06- 1.96 (m, 3H), 1.90-1.72 (m, 4H), 1.50-1.42 (m, 1H), ¹⁹F NMR (376 MHz, DMSO-d₆) δ −140.32, −172.04. |
| 2 (A) | 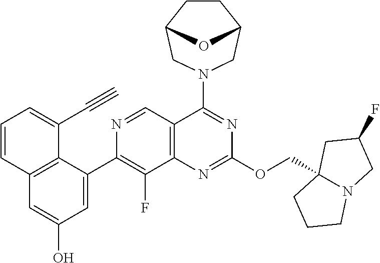 [SMILES: C#Cc1cccc2cc(O)cc(-c3ncc4c(N5C[C@H]6CC[C@@H](C5)O6)nc(OC[C@@]56CCCN5C[C@H](F)C6)nc4c3F)c12] 5-ethynyl-4-(8-fluoro-2- {[(2R,7aS)-2- fluorotetrahydro-1H- pyrrolizin-7a(5H)- yl]methoxy}-4-[(1R,5S)-8- oxa-3- azabicyclo[3.2.1]octan-3- yl]pyrido[4,3-d]pyrimidin-7- yl)naphthalen-2-ol | 584.2 | ¹H NMR (400 MHz, DMSO-d₆) δ 9.05 (s, 1H), 7.89-7.87 (m, 1H), 7.49-7.38 (m, 2H), 7.34-7.33 (m, 1H), 7.12-7.11 (m, 1H), 5.34-5.21 (m, 1H), 4.47-4.43 (m, 3H), 4.28- 4.25 (m, 1H), 4.12-4.00 (m, 2H), 3.74-3.64 (m, 2H), 3.59 (s, 1H), 3.18-3.04 (m, 2H), 3.03-3.01 (m, 1H), 2.87-2.80 (m, 1H), 2.13-2.12 (m, 1H), 2.05-1.97 (m, 2H), 1.91- 1.69 (m, 7H), ¹⁹F NMR (376 MHz, DMSO-d₆) δ −140.07, −172.13. |
| 3 (A) | 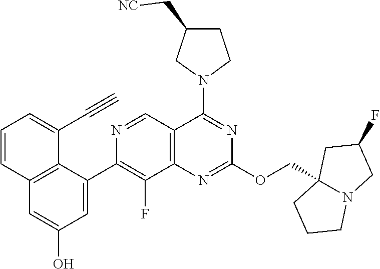 [SMILES: C#Cc1cccc2cc(O)cc(-c3ncc4c(N5CC[C@H](CC#N)C5)nc(OC[C@@]56CCCN5C[C@H](F)C6)nc4c3F)c12] {(3R)-1-[7-(8-ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]pyrrolidin- 3-yl}acetonitrile | 581.3 | ¹H NMR (400 MHz, DMSO-d₆) δ 10.16 (br s, 1H), 9.18 and 9.21 (2 s, 1H, major and minor rotamer respectively), 7.89-7.87 (m, 1H), 7.49-7.38 (m, 2H), 7.34-7.33 (m, 1H), 7.13-7.10 (m, 1H), 5.35-5.21 (m, 1H), 4.18-3.96 (m, 4H), 3.65- 3.58 (m, 2H), 3.10-3.02 (m, 3H), 2.87-2.83 (m, 3H), 2.70-2.67 (m, 1H), 2.33-2.27 (m, 2H), 2.12-2.01 (m, 3H), 1.92-1.71 (m, 4H), ¹⁹F NMR (376 MHz, DMSO-d₆) δ −140.04, −172.14. |
| 4 (A) | 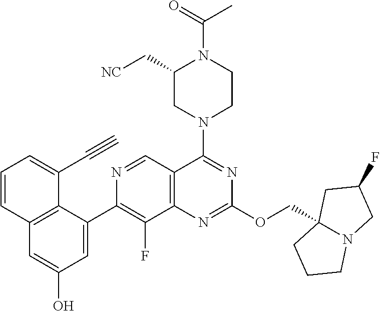 [SMILES: C#Cc1cccc2cc(O)cc(-c3ncc4c(N5CCN(C(C)=O)[C@@H](CC#N)C5)nc(OC[C@@]56CCCN5C[C@H](F)C6)nc4c3F)c12] {(2S)-1-acetyl-4-[7-(8- ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]piperazin- 2-yl}acetonitrile | 638.3 | ¹H NMR (400 MHz, DMSO-d₆) δ 9.13-9.08 (m, 1H), 7.89-7.87 (m, 1H), 7.46-7.39 (m, 2H), 7.35-7.34 (m, 1H), 7.16-7.12 (m, 1H), 5.37- 5.19 (m, 1H), 4.87-4.65 (m, 1H), 4.50-4.29 (m, 2H), 4.15-4.05 (m, 2H), 3.89-3.75 (m, 2H), 3.63-3.56 (m, 2H), 3.11-2.99 (m, 5H), 2.86- 2.80 (m, 1H), 2.18-2.00 (m, 7H), 1.86-1.77 (m, 3H), ¹⁹F NMR (376 MHz, DMSO-d₆) δ −140.24, −172.08 |
| 5 (A) | 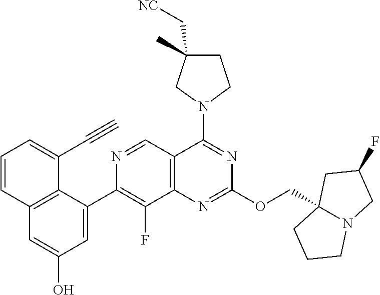 [SMILES: C#Cc1cccc2cc(O)cc(-c3ncc4c(N5CC[C@@](C)(CC#N)C5)nc(OC[C@@]56CCCN5C[C@H](F)C6)nc4c3F)c12] {(3S)-1-[7-(8-ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-3- methylpyrrolidin-3- yl}acetonitrile | 595.3 | ¹H NMR (400 MHz, MeOD-d₄) δ 9.35 (s, 1H), 7.89-7.87 (m, 1H), 7.57-7.55 (m, 1H), 7.47-7.42 (m, 2H), 7.28-7.27 (m, 1H), 5.65-5.52 (m, 1H), 4.82-4.73 (m, 2H), 4.39 (br s, 1H), 4.12-4.03 (m, 2H), 4.00- 3.83 (m, 4H), 3.49-3.41 (m, 1H), 2.81-2.60 (m, 4H), 2.49-2.17 (m, 7H), 1.43 (s, 3H), ¹⁹F NMR (376 MHz, MeOD-d4) δ 137.39, −174.19. |
| 6 (A) | 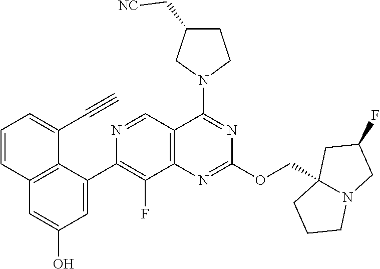 [SMILES: C#Cc1cccc2cc(O)cc(-c3ncc4c(N5CC[C@@H](CC#N)C5)nc(OC[C@@]56CCCN5C[C@H](F)C6)nc4c3F)c12] {(3S)-1-[7-(8-ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]pyrrolidin- 3-yl}acetonitrile | 581.2 | ¹H NMR (400 MHz, MeOD-d₄) δ 9.22 and 9.20 (2 s, 1H, two rotamers respectively), 7.84-7.82 (m, 1H), 7.53-7.51 (m, 1H), 7.42-7.39 (m, 1H), 7.34-7.33 (m, 1H), 7.22-7.14 (m, 1H), 5.39-5.25 (m, 1H), 4.40- 4.06 (m, 5H), 3.84 (br s, 1H), 3.30- 3.19 (m, 3H), 3.05-3.00 (m, 1H), 2.88-2.73 (m, 3H), 2.46-2.37 (m, 1H), 2.37-2.14 (m, 3H), 2.08-1.82 (m, 5H), ¹⁹F NMR (376 MHz, MeOD- d4) δ −139.48, −173.64. |
| 7 (A) | 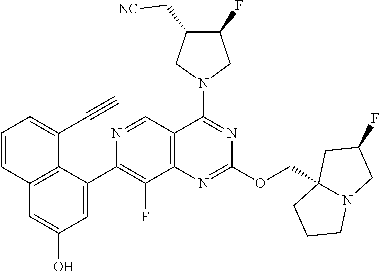 [SMILES: C#Cc1cccc2cc(O)cc(-c3ncc4c(N5C[C@H](CC#N)[C@@H](F)C5)nc(OC[C@@]56CCCN5C[C@H](F)C6)nc4c3F)c12] {(3SR,4RS)-1-[7-(8- ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-4- fluoropyrrolidin-3- yl}acetonitrile | 599.3 | ¹H NMR (400 MHz, DMSO-d₆) δ 9.23 and 9.22 (2 s, 1H, two rotamers respectively), 7.89-7.87 (m, 1H), 7.52-7.39 (m, 2H), 7.35-7.34 (m, 1H), 7.13-7.11 (m, 1H), 5.41-5.21 (m, 2H), 4.16-4.12 (m, 1H), 4.07- 4.02 (m, 1H), 3.95-3.91 (m, 1H), 3.59-3.54 (m, 3H), 3.14-3.02 (m, 5H), 2.86-2.75 (m, 3H), 2.13-1.95 (m, 3H), 1.88-1.74 (m, 3H), ¹⁹F NMR (376 MHz, DMSO-d₆) δ −139.64, −172.13. |
| 8 (A) | 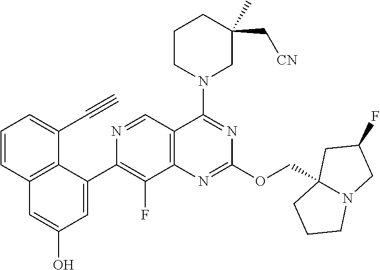 [SMILES: C#Cc1cccc2cc(O)cc(-c3ncc4c(N5CCC[C@@](C)(CC#N)C5)nc(OC[C@@]56CCCN5C[C@H](F)C6)nc4c3F)c12] {(3S)-1-[7-(8-ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-3- methylpiperidin-3- yl}acetonitrile | 609.3 | ¹H NMR (400 MHz, DMSO-d₆) δ 9.03 and 9.02 (2 s, 1H, two rotamers respectively), 7.88-7.87 (m, 1H), 7.47-7.36 (m, 2H), 7.34-7.33 (m, 1H), 7.15-7.14 (m, 1H), 5.34- 5.21 (m, 1H), 4.15-3.94 (m, 4H), 3.83-3.69 (m, 2H), 3.63-3.59 (m, 1H), 3.14-3.06 (m, 2H), 3.07-3.04 (m, 1H), 2.85-2.80 (m, 1H), 2.73- 2.67 (m, 2H), 2.13-1.95 (m, 4H), 1.86-1.64 (m, 6H), 1.15-1.13 (m, 3H), ¹⁹F NMR (376 MHz, DMSO-d₆) δ −140.45, −172.06. |
| 9 (A) | 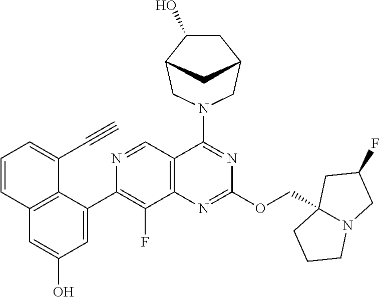 [SMILES: C#Cc1cccc2cc(O)cc(-c3ncc4c(N5C[C@@H]6C[C@H](C5)[C@H](O)C6)nc(OC[C@@]56CCCN5C[C@H](F)C6)nc4c3F)c12] (1R,5R,6R)-3-[7-(8- ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-3- azabicyclo[3.2.1]octan-6-ol | 598.3 | ¹H NMR (400 MHz, DMSO-d₆) δ 9.06 and 9.35 (2 s, 1H, major and minor rotamer respectively), 7.88- 7.86 (m, 1H), 7.44-7.38 (m, 2H), 7.34-7.33 (m, 1H), 7.19-7.11 (m, 1H), 5.35-5.21 (m, 1H), 5.06-5.03 (m, 1H), 4.65-4.59 (m, 1H), 4.43- 4.41 (m, 1H), 4.16-4.09 (m, 2H), 3.98-3.88 (m, 2H), 3.55-3.42 (m, 1H), 3.25-3.22 (m, 1H), 3.13-3.08 (m, 2H), 3.04-3.01 (m, 1H), 2.85- 2.81 (m, 1H), 2.33 (br s, 1H), 2.17- 1.95 (m, 6H), 1.87-1.71 (m, 4H), 1.68-1.63 (m, 1H), ¹⁹F NMR (376 MHz, DMSO-d₆) δ 141.20, −172.11. |
| 10 (B) | 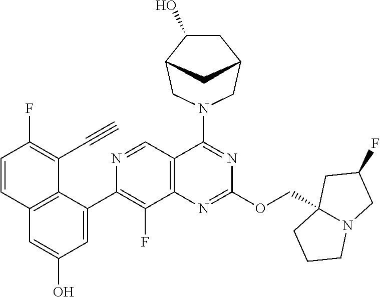 [SMILES: C#Cc1c(F)ccc2cc(O)cc(-c3ncc4c(N5C[C@@H]6C[C@H](C5)[C@H](O)C6)nc(OC[C@@]56CCCN5C[C@H](F)C6)nc4c3F)c12] (1R,5R,6R)-3-[7-(8- ethynyl-7-fluoro-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-3- azabicyclo[3.2.1]octan-6-ol | HRMS: 616.25385 | ¹H NMR (400 MHz, METHANOL- d4) δ 9.28-9.04 (m, 1H), 7.90-7.81 (m, 1H), 7.35 (d, J = 2.4 Hz, 1H), 7.34-7.28 (m, 1H), 7.27-7.19 (m, 1H), 5.49-5.30 (m, 1H), 5.21 (br d, J = 11.9 Hz, 1H), 4.67 (br d, J = 12.1 Hz, 1H), 4.50-4.42 (m, 1H), 4.38- 4.26 (m, 2H), 3.98-3.72 (m, 1H), 3.58-3.34 (m, 5H), 3.21-3.11 (m, 1H), 2.51-2.05 (m, 9H), 2.03-1.89 (m, 5H), 1.86-1.75 (m, 1H), 1.46- 1.36 (m, 1H). |
| 11 (B) | 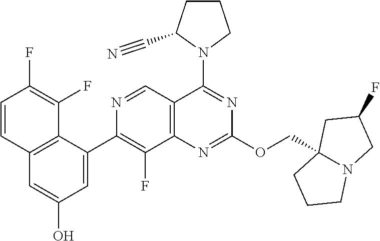 [SMILES: N#C[C@@H]1CCCN1c1nc(OC[C@@]23CCCN2C[C@H](F)C3)nc2c(F)c(-c3cc(O)cc4ccc(F)c(F)c34)ncc12] (2S)-1-[7-(7,8-difluoro-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]pyrrolidine- 2-carbonitrile | 579.0 | ¹H NMR (400 MHz, DMSO-d₆) δ 10.71 (br s, 1H), 9.34 (s, 1H), 7.74 (dd, J = 4.8, 8.7 Hz, 1H), 7.64-7.52 (m, 1H), 7.40 (s, 1H), 7.26 (br d, J = 4.9 Hz, 1H), 5.39-5.16 (m, 2H), 4.39-4.11 (m, 4H), 3.17-3.01 (m, 3H), 2.88-2.79 (m, 1H), 2.45-2.28 (m, 2H), 2.27-1.97 (m, 5H), 1.87- 1.75 (m, 3H), ¹⁹F NMR (376 MHz, DMSO-d₆) δ = −140.45 (br s, 1F), −143.18 (br dd, J = 20.6, 29.8 Hz, 1F), −144.46-−145.34 (m, 1F), −171.54-−172.47 (m, 1F) |
| 12 (B) | 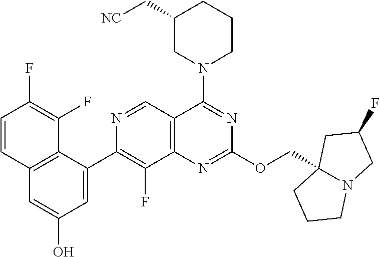 [SMILES: N#CC[C@@H]1CCCN(c2nc(OC[C@@]34CCCN3C[C@H](F)C4)nc3c(F)c(-c4cc(O)cc5ccc(F)c(F)c45)ncc23)C1] {(3S)-1-[7-(7,8-difluoro-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]piperidin-3- yl}acetonitrile | 607.2 | ¹H NMR (400 MHz, DMSO-d₆) δ 9.91 (br s, 1H) 9.10 (s, 1H) 7.70 (ddd, J = 9.17, 4.95, 1.53 Hz, 1H) 7.51 (td, J = 9.69, 7.76 Hz, 1H) 7.38 (t, J = 2.14 Hz, 1H) 7.26 (d, J = 2.08 Hz, 1H) 5.15-5.45 (m, 1H) 4.45- 4.55 (m, 1H) 4.33-4.44 (m, 1H) 4.11-4.32 (m, 2H) 3.48 (ddd, J = 13.45, 10.76, 3.06 Hz, 1H) 2.81- 3.37 (m, 7H) 2.56-2.75 (m, 2H) 1.70-2.32 (m, 9H) 1.41-1.62 (m, 1H). |
| 13 (B) | 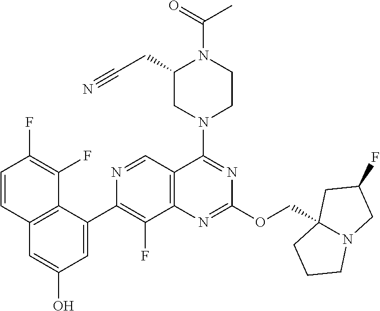 [SMILES: CC(=O)N1CCN(c2nc(OC[C@@]34CCCN3C[C@H](F)C4)nc3c(F)c(-c4cc(O)cc5ccc(F)c(F)c45)ncc23)C[C@@H]1CC#N] {(2S)-1-acetyl-4-[7-(7,8- difluoro-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]piperazin- 2-yl}acetonitrile | 650.0 | ¹H NMR (400 MHz, DMSO-d₆) δ 10.38-10.17 (m, 1H), 9.18 (s, 1H), 7.75 (br dd, J = 5.1, 8.8 Hz, 1H), 7.63-7.53 (m, 1H), 7.40 (s, 1H), 7.25 (br dd, J = 2.0, 10.0 Hz, 1H), 5.37-5.18 (m, 2H), 4.89-4.80 (m, 1H), 4.69-4.60 (m, 1H), 4.55-4.28 (m, 3H), 4.19-4.13 (m, 1H), 4.12- 4.05 (m, 1H), 3.93-3.71 (m, 2H), 3.17-2.93 (m, 4H), 2.89-2.76 (m, 1H), 2.18 (s, 1H), 2.16-2.11 (m, 1H), 2.09 (s, 1H), 2.06 (br d, J = 2.4 Hz, 1H), 2.03-1.98 (m, 1H), 1.91 (s, 1H), 1.88-1.73 (m, 2H), ¹⁹F NMR (376 MHz, DMSO-d₆) δ = −140.74- −141.08 (m, 1F), −142.80-−143.11 (m, 1F), −144.79-−145.07 (m, 1F), −172.05 (br d, J = 9.2 Hz, 1F). |
| 14 (B) | 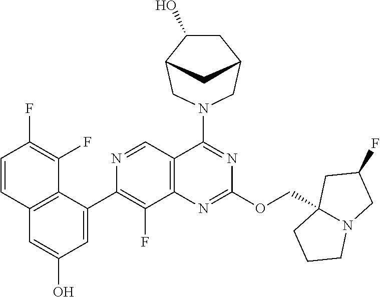 [SMILES: Oc1cc(-c2ncc3c(N4C[C@@H]5C[C@H](C4)[C@H](O)C5)nc(OC[C@@]45CCCN4C[C@H](F)C5)nc3c2F)c2c(F)c(F)ccc2c1] (1R,5R,6R)-3-[7-(7,8- difluoro-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-3- azabicyclo[3.2.1]octan-6-ol | 610.2 | ¹H NMR (400 MHz, DMSO-d₆) δ 9.35 (s, 1H) 7.70 (ddd, J = 9.11, 5.01, 1.53 Hz, 1H) 7.50 (td, J = 9.60, 7.70 Hz, 1H) 7.38 (t, J = 2.08 Hz, 1H) 7.26 (d, J = 2.20 Hz, 1H) 5.21- 5.53 (m, 1H) 4.70 (br d, J = 11.74 Hz, 2 H) 4.13-4.41 (m, 3 H) 3.68 (br dd, J = 12.47, 1.34 Hz, 1H) 3.10- 3.49 (m, 5 H) 2.90-3.04 (m, 1H) 1.59-2.45 (m, 10H) 1.22-1.44 (m, 1H). |
| 15 (B) | 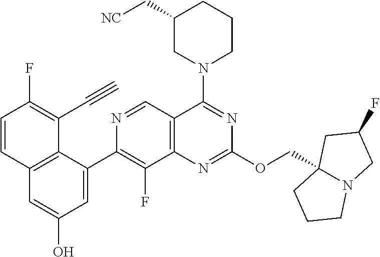 [SMILES: C#Cc1c(F)ccc2cc(O)cc(-c3ncc4c(N5CCC[C@@H](CC#N)C5)nc(OC[C@@]56CCCN5C[C@H](F)C6)nc4c3F)c12] {(3S)-1-[7-(8-ethynyl-7- fluoro-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]piperidin-3- yl}acetonitrile | 613.2 | ¹H NMR (400 MHz, DMSO-d₆) δ 10.26-10.00 (m, 1H), 9.00 (d, J = 9.0 Hz, 1H), 7.97 (dd, J = 5.9, 9.3 Hz, 1H), 7.46 (t, J = 9.0 Hz, 1H), 7.39 (d, J = 2.4 Hz, 1H), 7.19 (d, J = 2.4 Hz, 1H), 5.39-5.12 (m, 1H), 4.53-4.42 (m, 1H), 4.42-4.30 (m, 1H), 4.15 (dd, J = 7.6, 10.5 Hz, 1H), 4.08-3.99 (m, 1H), 3.96 (d, J = 2.4 Hz, 1H), 3.49-3.32 (m, 2H), 3.25- 2.97 (m, 4H), 2.89-2.77 (m, 1H), 2.74-2.58 (m, 2H), 2.24-2.08 (m, 2H), 2.06-1.94 (m, 3H), 1.91 (s, 2H), 1.57-1.39 (m, 1H), ¹⁹F NMR (376 MHz, DMSO-d₆) δ −110.72 (s, 1F), −140.22 (s, 0.5F), −140.36 (s, 0.5F), −171.99 (s, 0.5F), −172.10 (s, 0.5F). |
| 16 (B) | 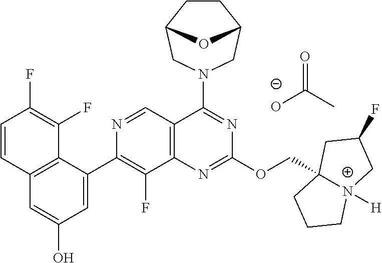 5,6-difluoro-4-(8-fluoro-2- {[(2R,7aS)-2- fluorotetrahydro-1H- pyrrolizin-7a(5H)- yl]methoxy}-4-[(1R,5S)-8- oxa-3- azabicyclo[3.2.1]octan-3- yl]pyrido[4,3-d]pyrimidin-7- yl)naphthalen-2-ol, acetic acid salt | 596.0 | ¹H NMR (400 MHz, DMSO-d₆) δ = 9.13 (s, 1H), 7.77-7.70 (m, 1H), 7.62-7.52 (m, 1H), 7.41-7.37 (m, 1H), 7.24 (d, J = 2.1 Hz, 1H), 5.39- 5.17 (m, 1H), 4.47 (br s, 2H), 4.44- 4.37 (m, 2H), 4.17-4.11 (m, 1H), 4.08-4.03 (m, 1H), 3.70 (br d, J = 12.0 Hz, 2H), 3.13-3.05 (m, 2H), 3.01 (s, 1H), 2.88-2.78 (m, 1H), 2.19-1.96 (m, 3H), 1.89 (s, 3H), 1.87 (s, 3H, OAc), 1.80-1.74 (m, 5H). |
| 17 (B) | 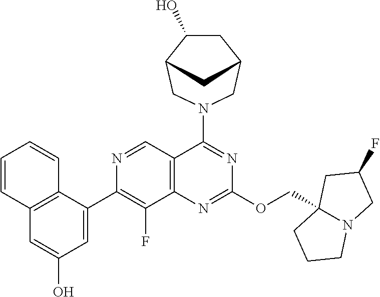 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccccc2c1] (1R,5R,6R)-3-[8-fluoro-2- {[(2R,7aS)-2- fluorotetrahydro-1H- pyrrolizin-7a(5H)- yl]methoxy}-7-(3- hydroxynaphthalen-1- yl)pyrido[4,3-d]pyrimidin-4- yl]-3- azabicyclo[3.2.1]octan-6-ol | 574.0 | ¹H NMR (400 MHz, DMSO-d₆) δ 9.60 (s, 1H) 9.41 (s, 1H) 7.78 (d, J = 8.19 Hz, 1H) 7.55 (br d, J = 7.8 Hz, 1H) 7.43 (td, J = 7.5, 1.1 Hz, 1H), 7.17-7.36 (m, 3H) 5.13-5.45 (m, 1H), 4.70 (br d, J = 12.3 Hz, 2H), 4.51 (d, J = 4.0 Hz, 1H), 4.18-4.28 (m, 2H), 4.05-4.17 (m, 1H), 3.67 (dd, J = 12.5, 2.1 Hz, 1H), 3.43 (br d, J = 12.7 Hz, 1H), 3.06-3.26 (m, 2H), 2.80-2.96 (m, 1H), 2.36 (br s, 1H), 1.97-2.27 (m, 5H), 1.65-1.95 (m, 5H), 1.22-1.43 (m, 1H). |
| 18^{a} (C) | 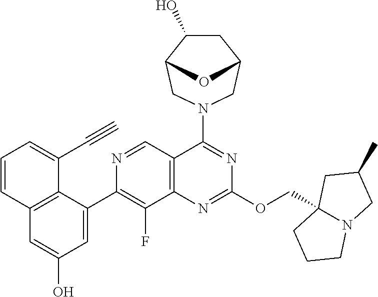 [SMILES: C#Cc1cccc2cc(O)cc(-c3ncc4c(N5C[C@H]6C[C@@H](O)[C@@H](C5)O6)nc(OC[C@@]56CCCN5C[C@H](C)C6)nc4c3F)c12] (1R*,5R*,6R*)-3-[7-(8- ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-8-oxa-3- azabicyclo[3.2.1]octan-6-ol | 600.2 | ¹H NMR (400 MHz, DMSO-d₆) δ 9.07 and 9.31 (2 s, 1H, major and minor rotamer respectively), 7.87 (dt, J = 8.0, 2.3 Hz, 1H), 7.46-7.39 (m, 2H), 7.34 (t, J = 2.5 Hz, 1H), 7.17 and 7.11 (2 d, J = 2.6 Hz, 1H, major and minor rotamer respectively), 5.26 (d, J = 54.3 Hz, 1H), 4.42 (d, J = 7.3 Hz, 1H), 4.33- 4.19 (m, 2H), 4.16-3.98 (m, 4H), 3.44 (d, J = 11.9 Hz, 1H), 3.07 (d, J = 9.5 Hz, 2H), 2.99 (s, 1H), 2.81 (t, J = 7.9 Hz, 1H), 2.34-2.25 (m, 1H), 2.11-2.02 (m, 2H), 1.77 (d, J = 8.2 Hz, 8H), ¹⁹F NMR (376 MHz, DMSO) δ −141.48, −172.18 (only major rotamers reported). All relative stereochemistry known; absolute stereochemistry unknown at the azabicyclo[3.2.1.] ring. |
| 19^{a} (C) | 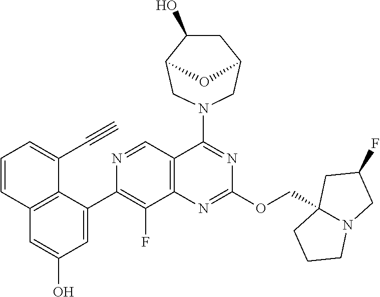 [SMILES: C#Cc1cccc2cc(O)cc(-c3ncc4c(N5C[C@@H]6C[C@H](O)[C@H](C5)O6)nc(OC[C@@]56CCCN5C[C@H](F)C6)nc4c3F)c12] (1S*,5S*,6S*)-3-[7-(8- ethynyl-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-8-oxa-3- azabicyclo[3.2.1]octan-6-ol | 600.2 | ¹H NMR (400 MHz, DMSO) δ 9.07 and 9.31 (2 s, 1H, major and minor rotamer respectively), 7.87 (dt, J = 8.0, 2.3 Hz, 1H), 7.46-7.39 (m, 2H), 7.34 (t, J = 2.5 Hz, 1H), 7.17 and 7.11 (2 d, J = 2.6 Hz, 1H, major and minor rotamer respectively), 5.26 (d, J = 54.3 Hz, 1H), 4.42 (d, J = 7.3 Hz, 1H), 4.33-4.19 (m, 2H), 4.16-3.98 (m, 4H), 3.44 (d, J = 11.9 Hz, 1H), 3.07 (d, J = 9.5 Hz, 2H), 2.99 (s, 1H), 2.81 (t, J = 7.9 Hz, 1H), 2.34-2.25 (m, 1H), 2.11-2.02 (m, 2H), 1.77 (d, J = 8.2 Hz, 8H), ¹⁹F NMR (376 MHz, DMSO) δ −141.48, −172.18 (only major rotamers reported). All relative stereochemistry known; absolute stereochemistry unknown at the azabicyclo[3.2.1.] ring. |
| 20 (C) | 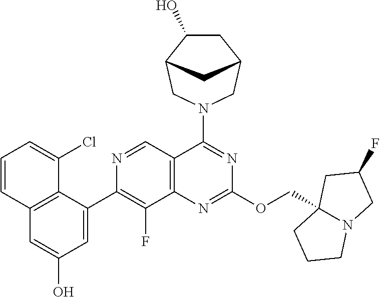 [SMILES: Oc1cc(-c2ncc3c(N4C[C@@H]5C[C@H](C4)[C@H](O)C5)nc(OC[C@@]45CCCN4C[C@H](F)C5)nc3c2F)c2c(Cl)cccc2c1] (1R,5R,6R)-3-[7-(8-chloro- 3-hydroxynaphthalen-1-yl)- 8-fluoro-2-{[(2R,7aS)-2- fluorotetrahydro-1H- pyrrolizin-7a(5H)- yl]methoxy}pyrido[4,3- d]pyrimidin-4-yl]-3- azabicyclo[3.2.1]octan-6-ol | 609.2 | ¹H NMR (400 MHz, DMSO-d₆) δ 9.28 (d, J = 24.6 Hz, 1H), 7.83 (dd, J = 8.2, 1.4 Hz, 1H), 7.43-7.33 (m, 3H), 7.14 (dd, J = 11.7, 2.5 Hz, 1H), 5.37-5.18 (m, 1H), 4.82-4.55 (m, 3H), 4.21-4.09 (m, 2H), 4.01 (dd, J = 10.4, 4.4 Hz, 1H), 3.75-3.65 (m, 1H), 3.38 (s, 1H), 3.12-3.06 (m, 2H), 3.01 (s, 1H), 2.82 (q, J = 8.2 Hz, 1H), 2.33 (d, J = 7.0 Hz, 1H), 2.17-1.98 (m, 5H), 1.87 (s, 2H), 1.81-1.73 (m, 3H), 1.67 (s, 1H), 1.26 (d, J = 13.4 Hz, 1H), ¹⁹F NMR (376 MHz, DMSO-d₆) δ −140.16, −140.66, −172.07, −172.12. |
| 21ª (C) | 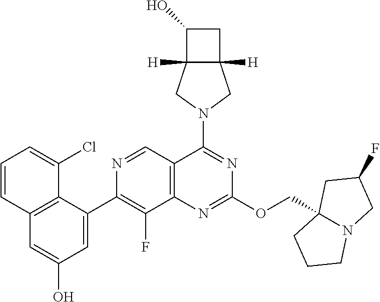 [SMILES: Oc1cc(-c2ncc3c(N4C[C@H]5C[C@@H](O)[C@H]5C4)nc(OC[C@@]45CCCN4C[C@H](F)C5)nc3c2F)c2c(Cl)cccc2c1] (1S*,5R*,6R*)-3-[7-(8- chloro-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-3- azabicyclo[3.2.0]heptan-6- ol | 594.2 | 1H NMR (400 MHz, DMSO) d 10.25 (d, J = 1.5 Hz, 1H), 9.32 (s, 1H), 7.84 (dd, J = 8.2, 1.4 Hz, 1H), 7.49- 7.28 (m, 3H), 7.14 (t, J = 2.8 Hz, 1H), 5.37 (d, J = 53.7 Hz, 1H), 5.20 (dd, J = 5.5, 3.6 Hz, 1H), 4.66-4.55 (m, 1H), 4.29 (s, 1H), 4.20 (p, J = 7.6 Hz, 3H), 3.95 (s, 2H), 3.18 (d, J = 17.2 Hz, 2H), 2.98 (s, 1H), 2.71 (t, J = 7.2 Hz, 1H), 2.54 (s, 1H), 2.34- 2.06 (m, 3H), 2.02-1.78 (m, 3H), 1.61-1.52 (m, 1H). 19F NMR (376 MHz, DMSO) d −136.95, −174.11. |
| 22ª (C) | 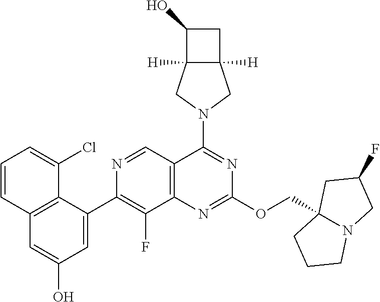 [SMILES: Oc1cc(-c2ncc3c(N4C[C@@H]5C[C@H](O)[C@@H]5C4)nc(OC[C@@]45CCCN4C[C@H](F)C5)nc3c2F)c2c(Cl)cccc2c1] (1R*,5S*,6S*)-3-[7-(8- chloro-3- hydroxynaphthalen-1-yl)-8- fluoro-2-{[(2R,7aS)-2- fluorotetrahydro-1H- pyrrolizin-7a(5H)- yl]methoxy}pyrido[4,3- d]pyrimidin-4-yl]-3- azabicyclo[3.2.0]heptan-6- ol | 594.2 | 1H NMR (400 MHz, DMSO) d 10.24 (d, J = 1.3 Hz, 1H), 9.32 (s, 1H), 7.84 (dd, J = 8.2, 1.4 Hz, 1H), 7.44- 7.34 (m, 3H), 7.14 (dd, J = 4.3, 2.6 Hz, 1H), 5.35 (d, J = 54.2 Hz, 1H), 5.19 (dd, J = 5.5, 3.6 Hz, 1H), 4.61 (d, J = 12.8 Hz, 1H), 4.32-4.12 (m, 4H), 3.94 (s, 2H), 3.37 (dd, J = 10.4, 5.7 Hz, 2H), 3.20 (s, 2H), 2.89 (s, 1H), 2.75-2.68 (m, 1H), 2.13 (d, J = 30.2 Hz, 3H), 1.90 (d, J = 41.2 Hz, 3H), 1.54 (d, J = 8.1 Hz, 1H), 1.26 (d, J = 18.2 Hz, 1H). 19F NMR (376 MHz, DMSO) d −138.08, −172.85 |

a. The symbol “*” found in (1R*, 5R*, 6R*) and (1S*, 5S*, 6S*) for the chemical names of Example 18 and Example 19 only indicates that the relative stereochemistry was known but the absolute chemistry was not for these two compounds. Therefore, (1R*, 5R*, 6R*) and (1S*, 5S*, 6S*) should not be considered the actual (1R, 5R, 6R) and (1S, 5S, 6S) such as found in Example 20, which has the determined absolute stereochemistry (1R, 5R, 6R).

### Example 23: 5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol

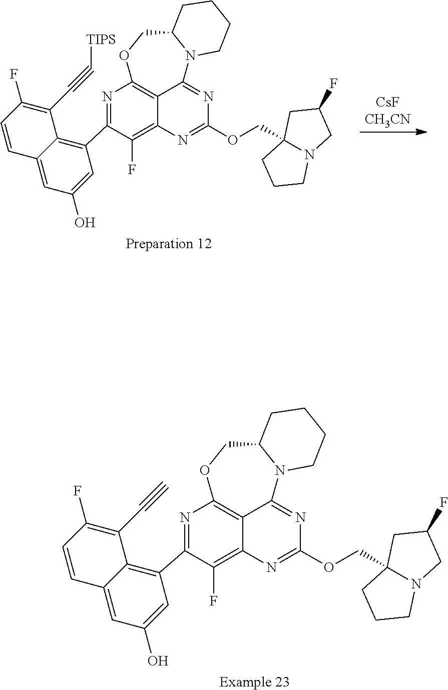

Preparation 12 (910 mg, 1.2 mmol) was dissolved in CH₃CN (12 mL) and CsF (1.1 g, 7.2 mmol) was added. The reaction was stirred at 35° C. for 16 h and the reaction was partitioned between water (15 mL) and EtOAc (30 mL). The water layer was extracted with EtOAc (20 mL×3) and the combined organic extract was dried over Na₂SO₄, filtered, and concentrated. The resulting solid was purified via SFC using a ZymorSPHER HADP 150×21.2 column and a gradient of 15-40% MeOH in CO₂, 100 mL/min, 110 bar to afford 493 mg (68%) of Example 23 as a yellow solid. ¹H NMR observed 29/30 protons (phenol exchangeable not observed)¹H NMR (400 MHz, DMSO) δ 7.89 (dd, J=8.8, 6.4 Hz, 1H), 7.40 (t, J=9.6 Hz, 1H), 7.30 (d, J=2.6 Hz, 1H), 7.13 (dd, J=14.8, 2.5 Hz, 1H), 5.28 (d, J=52.9 Hz, 1H), 5.20-5.06 (m, 1H), 4.53-4.36 (m, 2H), 4.12 (dd, J=10.4, 3.2 Hz, 1H), 4.05-3.97 (m, 2H), 3.92 (dd, J=9.5, 5.1 Hz, 1H), 3.13-3.06 (m, 2H), 3.01 (d, J=9.2 Hz, 2H), 2.83 (q, J=8.6 Hz, 1H), 2.20-2.09 (m, 1H), 2.03 (dd, J=20.6, 3.4 Hz, 2H), 1.92-1.47 (m, 9H), ¹⁹F NMR (377 MHz, DMSO) δ−111.58, −145.15, −172.11.

The steps and reaction conditions outlined above for Example 23 are hereby defined as General Method D.

Examples 24-31 reported in Table 1A were prepared according to General Method D/Example 23 with non-critical modifications that one skilled in the art would appreciate.

TABLE 1A
Example 24-31

| Ex. No. | | | |
|---|---|---|---|
| (Gen. | | LCMS | |
| Method) | Structure/IUPAC name | [M + H]⁺ | ¹H NMR; ¹⁹F NMR |
| 24 (D) | 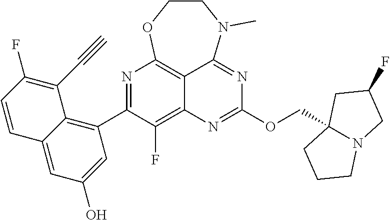 [SMILES: C#Cc1c(F)ccc2cc(O)cc(-c3nc4c5c(nc(OC[C@@]67CCCN6C[C@H](F)C7)nc5c3F)N(C)CCO4)c12] 5-ethynyl-6-fluoro-4-(4- fluoro-2-{[(2R,7aS)-2- fluorotetrahydro-1H- pyrrolizin-7a(5H)- yl]methoxy}-10-methyl- 9,10-dihydro-8H-7-oxa- 1,3,6,10- tetraazacyclohepta[1,2,3- de]naphthalen-5- yl)naphthalen-2-ol | 562.6 | ¹H NMR (400 MHz, DMSO) δ 10.13 (s, 1H), 7.96 (dd, J = 9.2, 5.9 Hz, 1H), 7.46 (t, J = 9.0 Hz, 1H), 7.38 (d, J = 2.6 Hz, 1H), 7.16, (d, J = 2.5 Hz, 1H), 4.56 (td, J = 5.2, 3.0 Hz, 2H), 4.04 (s, 1H), 4.03-3.94 (m, 2H), 3.39 (s, 3H); ¹⁹F NMR (377 MHz, DMSO) δ −110.76, −146.39. |
| 25 (D) | 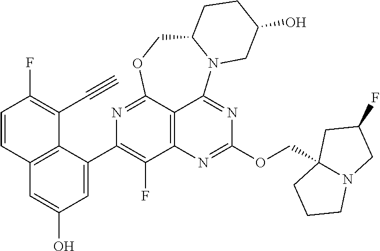 [SMILES: C#Cc1c(F)ccc2cc(O)cc(-c3nc4c5c(nc(OC[C@@]67CCCN6C[C@H](F)C7)nc5c3F)N3C[C@@H](O)CC[C@H]3CO4)c12] (8aS,11S)-5-(8-ethynyl-7- fluoro-3- hydroxynaphthalen-1-yl)-4- fluoro-2-{[(2R,7aS)-2- fluorotetrahydro-1H- pyrrolizin-7a(5H)- yl]methoxy}- 8,8a,9,10,11,12- hexahydro-7-oxa- 1,3,6,12a- tetraazabenzo[4,5]cyclohepta [1,2,3-de]naphthalen- 11-ol | 618.2 | ¹H NMR (600 MHz, DMSO-d₆) δ 10.12 (br d, J = 15.3 Hz, 1H), 7.96 (dd, J = 6.7, 9.1 Hz, 1H), 7.45 (q, J = 8.4 Hz, 1H), 7.37 (br s, 1H), 7.27- 7.07 (m, 1H), 5.29 (d, J = 54.5 Hz, 1H), 5.22-5.04 (m, 1H), 4.77-4.63 (m, 1H), 4.56-4.46 (m, 2H), 4.15- 4.09 (m, 1H), 4.10 (br s, 0.5H, alkyne CH), 3.80-4.00 (m, 3H), 3.65 (s, 0.5H, alkyne CH), 3.22 (br dd, J = 13.4, 18.9 Hz, 1H), 3.00- 3.10 (m, 3H), 2.84 (br s, 1H), 2.22- 2.00 (m, 4H), 1.89-1.74 (m, 5H), 1.64 (br s, 1H); ¹⁹F NMR (565 MHz, DMSO-d₆) δ −109.95-−110.97 (m, 1F), −145.30-−146.99 (m, 1F), −172.16 (br d, J = 56.2 Hz, 1F). |
| 26 (D) | 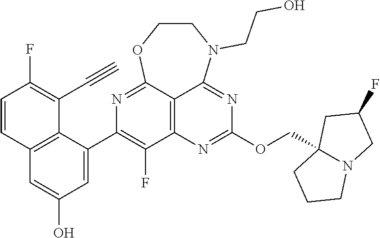 [SMILES: C#Cc1c(F)ccc2cc(O)cc(-c3nc4c5c(nc(OC[C@@]67CCCN6C[C@H](F)C7)nc5c3F)N(CCO)CCO4)c12] 5-ethynyl-6-fluoro-4-[4- fluoro-2-{[(2R,7aS)-2- fluorotetrahydro-1H- pyrrolizin-7a(5H)- yl]methoxy}-10-(2- hydroxyethyl)-9,10- dihydro-8H-7-oxa-1,3,6,10- tetraazacyclohepta[1,2,3- de]naphthalen-5- yl]naphthalen-2-ol | 592.2 | ¹H NMR (600 MHz, METHANOL-d₄) δ 7.85 (dd, J = 5.8, 9.1 Hz, 1H), 7.34 (d, J = 2.5 Hz, 1H), 7.32 (t, J = 8.9 Hz, 1H), 7.22 (br s, 1H), 5.50 (d, J = 52.1 Hz, 1H), 4.71-4.65 (m, 1H), 4.65-4.61 (m, 1H), 4.63-4.57 (m, 1H), 4.57-4.51 (m, 1H), 4.19-4.07 (m, 2H), 4.19-4.07 (m, 2H), 3.95 (t, J = 5.4 Hz, 2H), 3.91-3.77 (m, 1H), 3.78-3.73 (m, 1H), 3.76-3.68 (m, 1H), 3.53 (br s, 1H), 3.38-3.33 (m, 1H), 2.65-2.47 (m, 2H), 2.39-2.23 (m, 3H), 2.11-2.04 (m, 1H); ¹⁹F NMR (565 MHz, METHANOL-d₄) δ −111.62-−111.77 (m, 1F), −147.51 (br d, J = 24.1 Hz, 1F), −173.83-−174.37 (m, 1F). |
| 27 (D) | 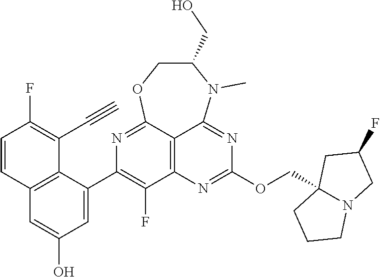 [SMILES: C#Cc1c(F)ccc2cc(O)cc(-c3nc4c5c(nc(OC[C@@]67CCCN6C[C@H](F)C7)nc5c3F)N(C)[C@@H](CO)CO4)c12] 5-ethynyl-6-fluoro-4-[4- fluoro-2-{[(2R,7aS)-2- fluorotetrahydro-1H- pyrrolizin-7a(5H)- yl]methoxy}-10-(2- hydroxyethyl)-9,10- dihydro-8H-7-oxa-1,3,6,10- tetraazacyclohepta[1,2,3- de]naphthalen-5- yl]naphthalen-2-ol | 592.2 | ¹H NMR (600 MHz, METHANOL-d₄) δ 7.88-7.79 (m, 1H), 7.32 (br s, 1H), 7.30 (t, J = 8.5 Hz, 1H), 7.27- 7.13 (m, 1H), 5.34 (br d, J = 53.8 Hz, 1H), 4.89-4.83 (m, 1H), 4.49-4.44 (m, 1H), 4.42-4.35 (m, 1H), 4.30 (q, J = 9.7 Hz, 1H), 4.11-4.05 (m, 1H), 3.93 (dt, J = 7.0, 11.6 Hz, 1H), 3.85- 3.80 (m, 1H), 3.54 (br s, 3H), 3.50- 3.24 (m, 4H), 3.09-3.04 (m, 1H), 2.41-2.23 (m, 2H), 2.18-2.13 (m, 1H), 2.06-2.01 (m, 2H), 1.95-1.88 (m, 1H), ¹⁹F NMR (565 MHz, METHANOL-d₄) δ −111.61-−111.94 (m, 1F), −145.94-−147.13 (m, 1F), −173.63-−173.82 (m, 1F). |
| 28 (D) | 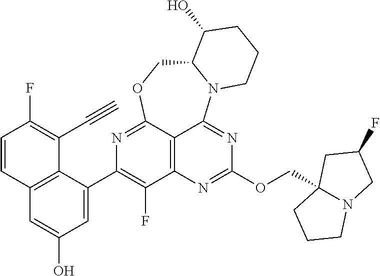 [SMILES: C#Cc1c(F)ccc2cc(O)cc(-c3nc4c5c(nc(OC[C@@]67CCCN6C[C@H](F)C7)nc5c3F)N3CCC[C@@H](O)[C@H]3CO4)c12] (8aR,9R)-5-(8-ethynyl-7- fluoro-3- hydroxynaphthalen-1-yl)-4- fluoro-2-{[(2R,7aS)-2- fluorotetrahydro-1H- pyrrolizin-7a(5H)- yl]methoxy}- 8,8a,9,10,11,12- hexahydro-7-oxa- 1,3,6,12a- tetraazabenzo[4,5]cyclohepta [1,2,3-de]naphthalen-9- ol | 618.3 | ¹H NMR (600 MHz, METHANOL-d₄) δ 7.83 (dd, J = 6.0, 8.9 Hz, 1H), 7.31 (d, J = 2.4 Hz, 1H), 7.29 (dd, J = 2.7, 9.1 Hz, 1H), 7.19 (dd, J = 2.5, 18.5 Hz, 1H), 5.31 (d, J = 50.7 Hz, 1H), 5.31-5.19 (m, 1H), 4.66-4.62 (m, 2H), 4.33 (t, J = 10.4 Hz, 1H), 4.21 (dd, J = 6.7, 10.4 Hz, 1H), 4.16 -4.10 (m, 1H), 4.13-4.08 (m, 1H), 3.51-3.35 (m, 1H), 3.28 (br s, 1H), 3.26-3.12 (m, 3H), 3.03-2.98 (m, 1H), 2.37-2.11 (m, 4H), 2.02-1.91 (m, 4H), 1.87 (br s, 1H), 1.67 (br d, J = 13.1 Hz, 1H); ¹⁹F NMR (565 MHz, METHANOL-d₄) δ −111.71- −112.02 (m, 1F), −145.24-−145.90 (m, 1F), −172.99-−174.03 (m, 1F) |
| 29 (D) | 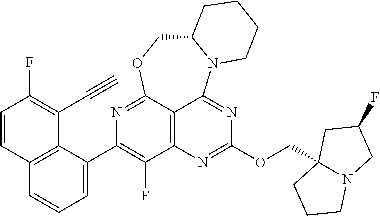 [SMILES: C#Cc1c(F)ccc2cccc(-c3nc4c5c(nc(OC[C@@]67CCCN6C[C@H](F)C7)nc5c3F)N3CCCC[C@H]3CO4)c12] (8aS)-5-(8-ethynyl-7- fluoronaphthalen-1-yl)-4- fluoro-2-{[(2R,7aS)-2- fluorotetrahydro-1H- pyrrolizin-7a(5H)- yl]methoxy}- 8,8a,9,10,11,12- hexahydro-7-oxa- 1,3,6,12a- tetraazabenzo[4,5]cyclohepta [1,2,3-de]naphthalene | 586.3 | ¹H NMR (400 MHz, METHANOL-d₄) δ 8.11-8.05 (m, 2H), 7.65-7.57 (m, 2H), 7.42 (td, J = 9.0, 1.3 Hz, 1H), 5.33 (t, J = 33.3 Hz, 2H), 4.87 (s, 1H), 4.60-4.45 (m, 2H), 4.24 (ddd, J = 33.1, 10.5, 6.5 Hz, 2H), 3.98 (d, J = 9.0 Hz, 1H), 3.57 (d, J = 11.6 Hz, 1H), 3.25-3.17 (m, 2H), 3.14-3.05 (m, 1H), 3.01 (dd, J = 15.1, 9.5 Hz, 1H), 2.38-2.18 (m, 2H), 2.17-2.09 (m, 1H), 2.00 (dd, J = 11.2, 5.0 Hz, 3H), 1.95-1.73 (m, 5H), 1.66 (d, J = 13.1 Hz, 1H). |
| 30 (D) | 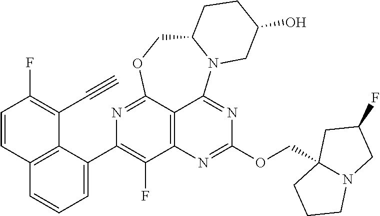 [SMILES: C#Cc1c(F)ccc2cccc(-c3nc4c5c(nc(OC[C@@]67CCCN6C[C@H](F)C7)nc5c3F)N3C[C@@H](O)CC[C@H]3CO4)c12] (8aS,11S)-5-(8-ethynyl-7- fluoronaphthalen-1-yl)-4- fluoro-2-{[(2R,7aS)-2- fluorotetrahydro-1H- pyrrolizin-7a(5H)- yl]methoxy}- 8,8a,9,10,11,12- hexahydro-7-oxa- 1,3,6,12a- tetraazabenzo[4,5]cyclohepta [1,2,3-de]naphthalen- 11-ol | 602.3 | ¹H NMR (400 MHz, DMSO) δ 8.26- 8.13 (m, 2H), 7.76-7.54 (m, 3H), 5.37-5.18 (m, 1H), 5.18-5.05 (m, 1H), 4.70 (dd, J = 29.1, 3.6 Hz, 1H), 4.51 (m, 2H), 4.18 (s, 0.5H), 4.11 (d, J = 10.3 Hz, 1H), 3.96 (dd, J = 10.3, 3.7 Hz, 2H), 3.83-3.90 (m, 1H), 3.72 (s, 0.5H), 3.22-3.17 (m, 1H), 3.05- 3.15 (m, 2H), 3.02 (s, 1H), 2.83 (q, J = 8.0 Hz, 1H), 2.21-1.93 (m, 4H), 1.71-1.83 (m, 4H), 1.65 (d, J = 13.0 Hz, 1H), ¹⁹F NMR (376 MHz, DMSO) δ −105.26, −105.75, −145.39, −146.50, −172.10, −172.20. |
| 31 (D) | 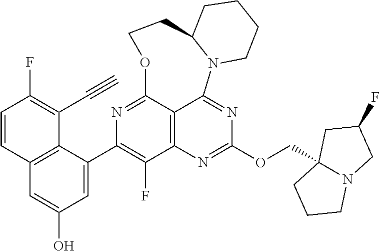 5-ethynyl-6-fluoro-4- [(6aR)-1-fluoro-12- {[(2R,7aS)-2- fluorotetrahydro-1H- pyrrolizin-7a(5H)- yl]methoxy}-6,6a,7,8,9,10- hexahydro-5H-4-oxa- 3,10a,11,13- tetraazabenzo[4,5]cycloocta [1,2,3-de]naphthalen-2- yl]naphthalen-2-ol | 616.2 | ¹H NMR (400 MHz, METHANOL-d₄) δ 7.83 (dd, J = 9.2, 5.7 Hz, 1H), 7.40- 7.03 (m, 3H), 5.51-5.12 (m, 2H), 4.60-4.37 (m, 2H), 4.35-4.07 (m, 3H), 3.52-3.37 (m, 4H), 3.23-3.07 (m, 2H), 2.56-2.40 (m, 2H), 2.39- 2.28 (m, 1H), 2.24-2.15 (m, 1H), 2.14-2.01 (m, 3H), 2.00-1.86 (m, 3H), 1.86-1.69 (m, 3H), 1.68-1.54 (m, 1H); ¹⁹F NMR (376 MHz, METHANOL-d₄) δ −111.66, −149.81, −173.82. |

### Example 32: 5-ethynyl-6,7-difluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol

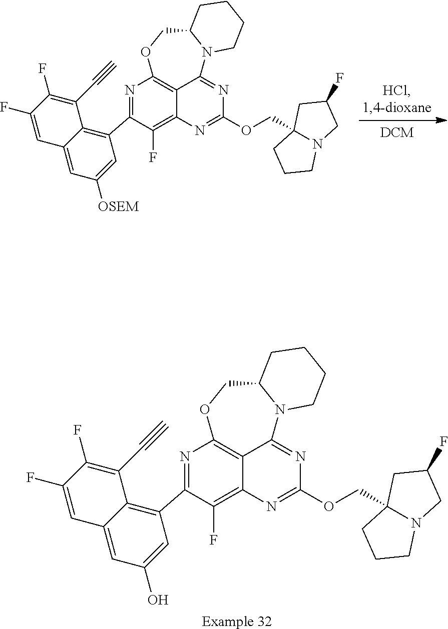

Using General Method D/Example 23 with non-critical modifications that one skilled in the art would appreciate, boronic ester Preparation 13 was coupled to Preparation 10 to afford Example 32 in 5 steps, for which the final SEM deprotection step is provided below. The SEM-protected intermediate leading to Example 32 (90 mg, 0.12 mmol) was dissolved in DCM (5 mL). To the DCM solution was added HCl in dioxane (0.45 mL of 4 M, 1.8 mmol) at 15° C. The reaction mixture was stirred at 15° C. under N₂ for 30 min. LCMS analysis showed that the starting material was almost consumed. The mixture was concentrated under vacuum to give the crude product which was purified using prep-HPLC (Waters MS triggered Prep-LC with SQD2 detector; column: Welch 10m C18 250×21.2 mm; flow rate: 25 mL/min; wavelength: 214 nm; 50% ~70% ACN in H₂O (0.1% NH₃) to give Example 32 (24 mg, 30%) as a yellow solid. ¹H NMR (METHANOL-d4, 400 MHz) d 7.6-7.7 (m, 1H), 7.25 (d, 1H, J=2.6 Hz), 7.15 (dd, 1H, J=2.5, 16.7 Hz), 5.2-5.4 (m, 2H), 4.4-4.6 (m, 2H), 4.2-4.4 (m, 1H), 4.1-4.2 (m, 1H), 3.9-4.0 (m, 1H), 3.68 (d, 1H, J=9.8 Hz), 3.1-3.3 (m, 3H), 3.0-3.2 (m, 1H), 3.0-3.0 (m, 1H), 2.3-2.4 (m, 1H), 2.2-2.4 (m, 1H), 2.1-2.2 (m, 1H), 1.7-2.1 (m, 8H), ¹⁹F NMR (METHANOL-d4, 376 MHz) d ⁻135.94-⁻136.00 (m, 1F), ⁻139.86-⁻139.91 (m, 1F), ⁻145.47-⁻145.64 (m, 1F), ⁻173.61-⁻173.67 (m, 1F), MS: 620 [M+H]⁺.

### Example 33: 5-ethynyl-6,7-difluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol

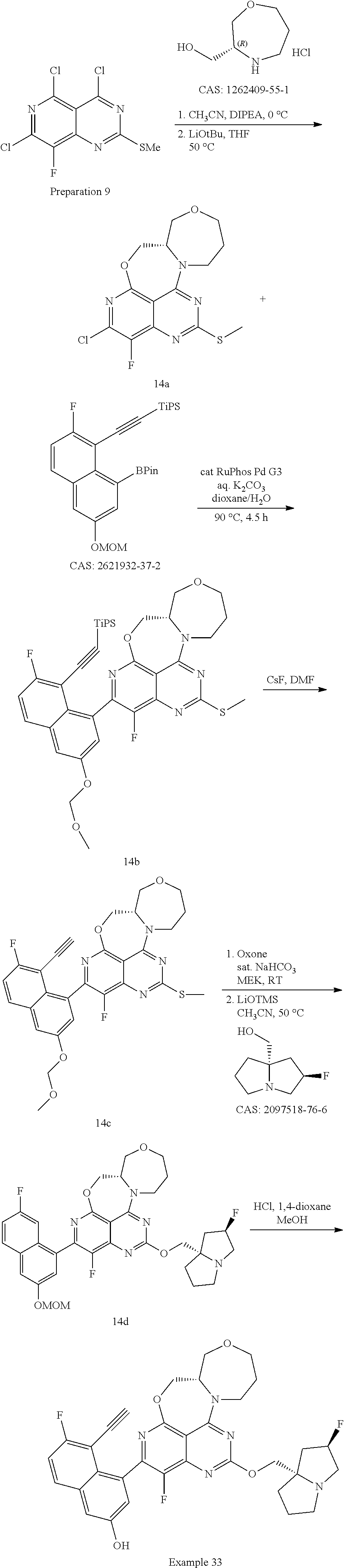

Preparation 9 (450 mg, 1.51 mmol) was suspended in CH₃CN (10 mL). DIPEA (276 uL, 1.59 mmol) was added and the suspension cooled to 0° C. under N₂. In a separate vial, CAS 1262409-55-1-HCl salt (232 mg, 1.39 mmol) was suspended in DCM (1 mL) and DIPEA (276 uL, 1.59 mmol) was added to dissolve the amine-HCl salt. THE (6 mL) was added to the resulting solution to give a milky mixture. This solution was added to the flask containing the cold solution of Preparation 9. After about 45 m at 0° C., LCMS analysis showed that the initial reaction was complete. LiOtBu (4.5 mL of 1 M in THF, 4.5 mmol) was added dropwise and the ice bath was removed. The ice bath was replaced with an oil bath and the reaction was heated at 50° C. for 30 min. LCMS analysis showed the cyclization step to be complete. The solution was cooled to rt and evaporated. Saturated aqueous NaHCO₃ (10 mL) was added and the mixture was extracted with DCM (3×30 mL). The combined organic extract was dried over Na₂SO₄ and evaporated. The process described above was repeated a second time on the same scale with the same observations and results. The crude material from both reactions was combined and purified using flash chromatography eluting with a gradient of 0-100% EtOAc in heptane and using DCM to load the crude material onto the silica cartridge. Fractions containing the desired product were pooled and concentrated to afford 483 mg of 14a (70%) as a tan solid. ¹H NMR (400 MHz, CHLOROFORM-d) δ=5.22 (ddd, J=2.9, 6.8, 13.8 Hz, 1H), 4.65 (dd, J=4.6, 13.4 Hz, 1H), 4.42 (d, J=13.4 Hz, 1H), 4.22-4.17 (m, 1H), 4.09-3.98 (m, 2H), 3.72 (dd, J=9.8, 12.6 Hz, 1H), 3.43-3.24 (m, 2H), 2.62 (s, 3H), 2.26-2.12 (m, 1H), 2.05-1.92 (m, 1H). ¹⁹F NMR (376 MHz, CHLOROFORM-d) δ=−140.51 (s, 1F). Analysis of this material by chiral SFC indicated optical purity of 50% ee indicating that the starting aminoalcohol (CAS 1262409-55-1-HCl salt) was not optically pure. Later in the sequence, the minor enantiomer was removed using preparative chiral SFC (vide infra). To a vial with a stir bar were added 14a (400 mg, 1.12 mmol), CAS 2621932-37-2 (689 mg, 1.35 mmol), 1,4-dioxane (5.6 mL) and K₂CO₃ (465 mg, 3.4 mmol) added as a solution in water (0.6 mL). The mixture was purged with N₂ for 3 min and RuPhos Pd G3 (94 mg, 0.11 mmol) was added. The vial was capped and heated at 90° C. for 4.5 h. LCMS showed that the starting material had been consumed and water (5 mL) was added. The dark mixture was extracted with EtOAc (3×30 mL). The combined organic extract was dried over Na₂SO₄, filtered, evaporated and submitted for chiral SFC purification to remove the minor enantiomer. After chiral SFC, 480 mg (62%) of 14b was obtained with 90% ee. ¹H NMR (400 MHz, METHANOL-d4) δ=7.95 (dd, J=5.8, 9.1 Hz, 1H), 7.64 (d, J=2.5 Hz, 1H), 7.40 (t, J=8.9 Hz, 1H), 7.34 (dd, J=2.3, 19.4 Hz, 1H), 5.39-5.30 (m, 2H), 5.26-5.14 (m, 1H), 4.71 (ddd, J=4.7, 13.6, 18.6 Hz, 1H), 4.57-4.48 (m, 1H), 4.31 (tt, J=4.9, 9.4 Hz, 1H), 4.24-4.14 (m, 1H), 4.06-3.79 (m, 2H), 3.71-3.52 (m, 2H), 3.51-3.48 (m, 3H), 2.61 (s, 3H), 2.28-2.12 (m, 1H), 2.07-1.88 (m, 1H), 0.98-0.90 (m, 18H), 0.77-0.61 (m, 3H), ¹⁹F NMR (376 MHz, METHANOL-d4) δ=−108.33 (d, J=69.4 Hz, 1F), −141.66-−144.80 (m, 1F), MS: 707 [M+H]⁺. 14b (402 mg, 0.57 mmol) was dissolved in DMF (9.5 mL). Anhydrous CsF (518 mg, 3.42 mmol) was added and the reaction was heated to 35° C. for 45 min. Most of the DMF was removed in vacuo (5 mm Hg, 30° C.) and water (5 mL) was added to the resulting residue. A solid precipitated out which was extracted into DCM (3×20 mL). The combined organic extract was dried over Na₂SO₄ filtered and evaporated to afford the crude material which was submitted for chiral SFC to further enrich in the desired enantiomer. After chiral SFC, 257 mg of 14c (82%) was obtained as a tan solid. ¹H NMR (400 MHz, CHLOROFORM-d) δ=7.87-7.74 (m, 1H), 7.55-7.50 (m, 1H), 7.46-7.35 (m, 1H), 5.37-5.16 (m, 3H), 4.71-4.59 (m, 1H), 4.54-4.41 (m, 1H), 4.21 (dt, J=3.8, 12.2 Hz, 1H), 4.14-3.97 (m, 2H), 3.83 (td, J=9.1, 12.6 Hz, 1H), 3.52 (d, J=2.8 Hz, 3H), 3.47-3.23 (m, 2H), 2.92 (d, J=19.6 Hz, 1H), 2.64 (s, 3H), 2.36-2.17 (m, 1H), 2.13-1.93 (m, 1H), 1.50 (s, 1H), ¹⁹F NMR (376 MHz, CHLOROFORM-d) δ=−106.89-−109.75 (m, 1F), −143.05 (d, J=363.4 Hz, 1F). To a mixture of 14c (252 mg, 0.46 mmol) in MEK (7 mL) was added solid Oxone (646 mg, 1.03 mmol), and saturated aqueous NaHCO₃ (7 mL). The mixture was stirred at 1500 rpm at rt for 20 min. The reaction was diluted with EtOAc (20 mL) and the aqueous layer was extracted twice more with EtOAc (2×20 mL). The combined organic extract was washed with 10% aqueous Na₂S₂O₃ (10 mL) and brine (10 mL). After drying over Na₂SO₄, the mixture was filtered and evaporated to afford 301 mg of the sulfone as a light-yellow gum. MS: 583 [M+H]⁺. This material was taken to the next step without further purification. The sulfone (267 mg, 0.458 mmol), and CAS 2097518-76-6 (100 mg, 0.64 mmol) were dissolved in CH₃CN (0.9 mL). Lithium trimethylsilanolate (130 mg, 1.4 mmol) was added and the vial was capped, stirred and heated to 50° C. for 30 min to afford 14d which was taken on to the MOM deprotection step in the same pot. After cooling to rt, MeOH (2 mL) was added to the 14d solution followed by HCl (3.5 mL of 4 M in 1,4-dioxane, 14 mmol) and the reaction was stirred at rt for 30 min. The solvents were removed under vacuum and saturated aqueous NaHCO₃ (10 mL) and water (10 mL) were added. A solid precipitated out from this mixture which was not soluble in EtOAc but was soluble in DCM. The aqueous mixture was extracted with DCM 1×150 mL and then 2×50 mL. The combined organic extract was dried over Na₂SO₄, filtered, evaporated and submitted for purification. Purification was accomplished using SFC with a Princeton HA-Morpholine 5 μm 21.2×150 mm column eluting with CO2/MeOH 10-50% in 5.0 minutes, 120 bar, 100 mL/min to afford 193 mg (68%) of Example 33 as a white solid. ¹H NMR (400 MHz, METHANOL-d4) δ=7.83 (dd, J=5.7, 9.1 Hz, 1H), 7.32 (d, J=2.5 Hz, 1H), 7.32-7.27 (m, 1H), 7.25-7.15 (m, 1H), 5.41-5.17 (m, 2H), 4.72 (td, J=4.6, 13.5 Hz, 1H), 4.61-4.53 (m, 1H), 4.41-4.22 (m, 3H), 4.22-4.11 (m, 1H), 4.04-3.92 (m, 1H), 3.82 (ddd, J=5.3, 9.8, 12.3 Hz, 1H), 3.71-3.51 (m, 1H), 3.50 and 3.36 (alkyne H has 2 chemical shifts, both doublets with, J=0.8 Hz, 1H), 3.49-3.38 (m, 1H), 3.28-2.95 (m, 4H), 2.42-2.09 (m, 4H), 2.06-1.81 (m, 4H); ¹⁹F NMR (377 MHz, METHANOL-d4) δ=−111.68 (qd, J=4.6, 61.0 Hz, 1F), ⁻143.97-⁻147.51 (m, 1F), ⁻173.14-⁻174.37 (m, 1F), MS: 618 [M+H]⁺.

### Example 34 ({5-ethynyl-6-fluoro-4-[(8aS)-4-fluoro-2-{[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalen-5-yl]naphthalen-2-yl}oxy)methyl dihydrogen phosphate

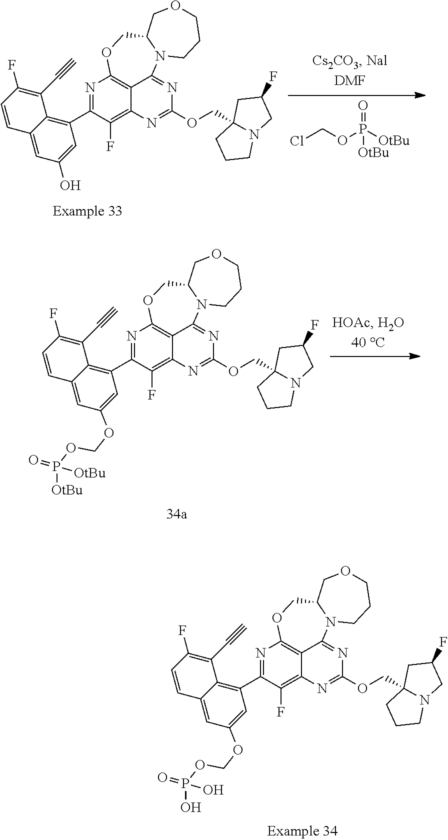 [SMILES: C#Cc1c(F)ccc2cc(O)cc(-c3nc4c5c(nc(OC[C@@]67CCCN6C[C@H](F)C7)nc5c3F)N3CCCOC[C@H]3CO4)c12 ; CC(C)(C)OP(=O)(OCCl)OC(C)(C)C ; C#Cc1c(F)ccc2cc(OCOP(=O)(OC(C)(C)C)OC(C)(C)C)cc(-c3nc4c5c(nc(OC[C@@]67CCCN6C[C@H](F)C7)nc5c3F)N3CCCOC[C@H]3CO4)c12 ; C#Cc1c(F)ccc2cc(OCOP(=O)(O)O)cc(-c3nc4c5c(nc(OC[C@@]67CCCN6C[C@H](F)C7)nc5c3F)N3CCCOC[C@H]3CO4)c12]

Example 33 (850 mg, 1.3 mmol) was dissolved in DMF (12.6 mL). Cesium carbonate (1.24 g, 3.79 mmol) and NaI (569 mg, 3.79 mmol) were added followed by di-tert-butyl chloromethyl phosphate (360 mg, 1.39 mmol). The mixture was stirred at 20° C. for 22 h. LCMS analysis showed 90% conversion to 34a and another portion of di-tert-butyl chloromethyl phosphate (72 mg, 0.14 mmol) was added the mixture stirred at 20° C. for an additional 5 h. Water (44 mL) was added and the mixture stirred for 40 min at 20° C. Multiple stir bars were used to adjust to smaller particle size. The solids were filtered, collected, and placed on high vacuum overnight to afford 34a (936 mg, 88%), MS: 840.3 [M+H]⁺. 34a (936 mg, 1.11 mmol) was dissolved in a mixture of acetic acid (9.0 mL) and deionized water (4.5 mL). The mixture was stirred at 40° C. for 16 h at which point LCMS analysis showed hydrolysis of the tert-butyl groups. The mixture was evaporated and re-dissolved in 50/50 CH₃CN/water (6 mL) and purified by HPLC. Desired fractions were collected to afford Example 34 (560 mg, 69%). The solid was placed under vacuum for 3 days to remove the acetic acid. ¹H NMR (400 MHz, DMSO) δ 8.03 (ddd, J=8.6, 5.9, 2.4 Hz, 1H), 7.75 (t, J=2.7 Hz, 1H), 7.51 (t, J=9.0 Hz, 1H), 7.37 (dd, J=41.4, 2.6 Hz, 1H), 5.60 (d, J=10.5 Hz, 2H), 5.40 (d, J=53.5 Hz, 1H), 5.04-4.94 (m, 1H), 4.66 (dt, J=13.4, 4.9 Hz, 1H), 4.52-4.47 (m, 1H), 4.34 (p, J=10.8, 10.3 Hz, 3H), 4.14-4.11 (m, 1H), 3.91-3.85 (m, 1H), 3.72-3.51 (m, 2H), 3.47-3.38 (m, 4H), 3.03 (s, 1H), 2.41-2.26 (m, 2H), 2.21-1.82 (m, 6H), MS: 728.2 [M+H]⁺.

Example 34 was developed as a prodrug of Example 33 to improve the unbound exposure of Example 33 in plasma.

The solution formulation of Example 33 was an aqueous solution with 2.5% (w/v) Pluronic F-68 (Poloxamer 188). The amorphous suspension formulations of Example 33 and Example 34 were made using in 0.5% (w/v) methylcellulose in water.

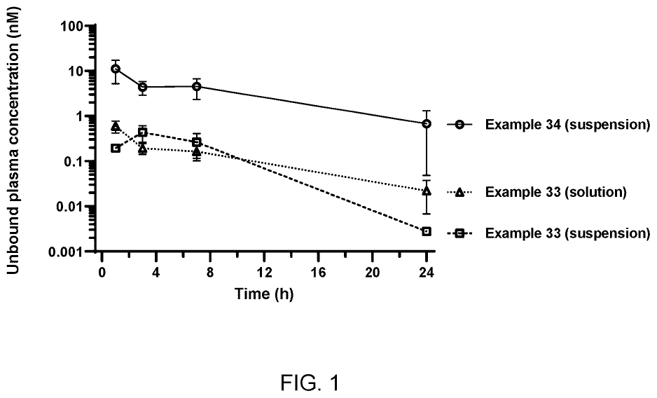

As demonstrated in FIG. 1 and Table 1-A, following single-dose oral administration of 100 mg (active)/kg in female NSG mice, mean systemic exposure of Example 33 (as assessed by AUC and/or Cav) was approximately 10-17 fold higher following the administration of Example 34 relative to administration of an oral suspension dose of Example 33 (free base).

TABLE 1-A

| | Cmax, u | Cav, ss, u | Cmax Fold | Cav Fold |
|---|---|---|---|---|
| Form/Formulation | (nM) | (nM) | increase | Increase |
| Example 33 - | 0.60 | 0.30 | — | — |
| solution | | | | |
| Example 33 - | 0.48 | 0.37 | 1 | 1 |
| suspension | | | | |
| Example 34 - | 8.12 | 3.84 | 16.9 | 10.4 |
| suspension | | | | |

### Prophetic Deuterated Analogs (PDA) of Example 33

The compounds provided in Table 2 are prophetic deuterated analogs (PDA) of Example 33. The Formula (V) is the generic formula of deuterated Example 33, wherein Y^{1a}, Y^{1b}, Y^{2a}, Y^{2b}, Y^{3a}, Y^{3b}, Y⁴ and Y⁵ are each independently H or D. The deuterated analogs of Example 33 in Table 2 are predicted based on the metabolic profile of Example 33 with MetaSite (moldiscovery.com/software/metasite/). Y^{1a}, Y^{1b}, Y^{2a}, Y^{2b}, Y^{3a}, Y⁴ and Y⁵ are most likely to be metabolized position based on MetaSite predictions.

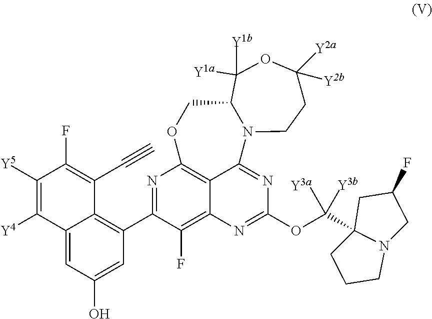

TABLE 2

| PDA # | Y^{1a} | Y^{1b} | Y^{2a} | Y^{2b} | Y^{3a} | Y^{3b} | Y⁴ | Y⁵ |
|---|---|---|---|---|---|---|---|---|
| V-1 | D | H | H | H | H | H | H | H |
| V-2 | H | D | H | H | H | H | H | H |
| V-3 | D | D | H | H | H | H | H | H |
| V-4 | H | H | D | H | H | H | H | H |
| V-5 | H | H | H | D | H | H | H | H |
| V-6 | H | H | D | D | H | H | H | H |
| V-7 | H | H | H | H | D | H | H | H |
| V-8 | H | H | H | H | H | D | H | H |
| V-9 | H | H | H | H | D | D | H | H |
| V-10 | H | H | H | H | H | H | D | H |
| V-11 | H | H | H | H | H | H | H | D |
| V-12 | D | D | D | D | H | H | H | H |
| V-13 | D | D | H | H | D | D | H | H |
| V-14 | H | H | D | D | D | D | H | H |
| V-15 | D | D | H | H | H | H | H | D |
| V-16 | H | H | D | D | H | H | H | D |
| V-17 | H | H | H | H | D | D | H | D |
| V-18 | H | H | H | H | H | H | D | D |

General methods/reviews of obtaining metabolite profile and identifying metabolites of a compound are described in: Dalvie, et al., “Assessment of Three Human in Vitro Systems in the Generation of Major Human Excretory and Circulating Metabolites,” Chemical Research in Toxicology, 2009, 22, 2, 357-368, tx8004357 (acs.orq); King, R., “Biotransformations in Drug Metabolism,” Ch. 3, Drug Metabolism Handbook Introduction, https://doi.org/10.1002/9781119851042.ch3; Wu, Y., et al, “Metabolite Identification in the Preclinical and Clinical Phase of Drug Development,” Current Drug Metabolish, 2021, 22, 11, 838-857, 10.2174/1389200222666211006104502; Godzien, J., et al, “Chapter Fifteen—Metabolite Annotation and Identification”.

Numerous publicly available and commercially available software tools are available to aid in the predictions of metabolic pathways and metabolites of compounds. Examples of such tools include, BioTransofrmer 3.0 (biotransformer.ca/new) which predicts the metabolic biotransformations of small molecules using a database of known metabolic reactions; MetaSite (moldiscovery.com/software/metasite/) which predicts metabolic transformations related to cytochrome P450 and flavin-containing monooxygenase mediated reactions in phase I metabolism; and Lhasa Meteor Nexus (lhasalimited.org/products/meteor-nexus.htm) offers prediction of metabolic pathways and metabolite structures using a range of machine learning models, which covers phase I and phase II biotransformations of small molecules.

Predicted deuterated analogs V-1 to V-18 of Example 33 in Table 2 may afford certain therapeutic advantages resulting from greater metabolic stability, for example increased in vivo half-life, reduced dosage requirements, reduced CYP450 inhibition (competitive or time dependent), or an improvement in therapeutic index or tolerability.

A person with ordinary skill may make additional deuterated analogs of Example 33 with different combinations of Y^{1a}, Y^{1b}, Y^{2a}, Y^{2b}, Y^{3a}, Y^{3b}, Y⁴ and Y⁵. Such additional deuterated analogs may provide similar therapeutic advantages that may be achieved by the deuterated analogs V-1 to V-18 of Example 33 in Table 2.

### KRAS Surface Plasma Resonance (SPR) Binding Assay

SPR assay was used to measure the kinetic binding constant (K_{D}) of Examples of the present invention.

The binding affinity and kinetics of Examples of the present invention were measured by Surface Plasmon Resonance (SPR) using Biacore 8K or 8K+ (Cytiva, Marlborough, MA) instruments. Recombinant, C-terminal site-specific biotinylated, wild-type (WT) KRAS (aa1-185), G12D KRAS (aa1-185), G12C KRAS (aa2-184), G12V KRAS (aa2-184), WT HRAS (aa2-184) and WT NRAS (aa2-185) proteins purified in presence of 1 μM GDP were used in these experiments. Binding measurements were performed parallelly in sets of either WT/G12D/G12C/G12V KRAS or WT K/H/N RAS proteins.

Biacore instrument was desorbed and docked with a Series S Sensor Chip SA. The proteins were diluted to 50 μg/mL with the assay buffer (50 mM HEPES, 150 mM NaCl, 10 μM GDP, 5 mM MgCl₂, 0.5 mM TCEP, 5% glycerol, 0.02% Tween-20, 2% DMSO, pH 7.2) and immobilized at a flow rate of 3 μL/min at 10° C. with a contact time of 15 min. to capture ~3000-4000 RUs of proteins on the surface. The functionalized surface was then equilibrated with assay buffer for approximately 1 hour. Un-functionalized SA surfaces with no immobilized protein served as reference for binding kinetic analysis. Compound binding kinetics were measured in either multi-cycle or single-cycle kinetic format.

### Multi-Cycle Kinetic Analysis (MCK)

A 2-fold, 10-point serial dilution of test compounds was set-up in a 96-well microplate (Greiner; Cat #650101) with a top concentration of either 10 μM or 100 μM. Binding kinetics was measured at 10° C. by injecting serial dilution of compounds onto both reference and RAS immobilized channels at a flow rate of 100 μL/min and association time of 90 seconds. Compound dissociation was monitored for at least 400 seconds during each cycle. No additional regeneration was used. DMSO calibration curve was obtained before and after compound analysis by injecting 0-4% of DMSO in assay buffer. A suitable compound with known affinity and kinetics was tested once in every experiment as a positive control to assess activity of the captured protein on the surface.

### Single-Cycle Kinetic Analysis (SCK)

A 3-fold, 6-point serial dilution of compounds was set-up in a deep 96-well microplate (Greiner Bio; Cat #780201) with the highest concentration of 1 μM (concentration range: 0.004-1 μM). Binding kinetics was measured at 10° C. by injecting serial dilutions of compounds in increasing order onto reference as well as RAS immobilized channels at a flow rate of 100 μL/min and association time of 120 seconds. Compound dissociation was monitored for at least 3600 seconds. Two buffer blanks were also run in a single-cycle kinetics format before the compound run for double referencing. No additional regeneration was used. DMSO calibration curve was obtained before and after compound analysis by injecting 0-4% of DMSO in the assay buffer. A suitable compound with known affinity and kinetics was tested once in every experiment as a positive control to assess activity of the captured protein on the surface.

Both MCK and SCK data were processed and analyzed using Biacore Insight evaluation software (Cytiva, Marlborough, MA)). The double-referenced and solvent-corrected data was fit to 1:1 Langmuir model to measure kinetic binding constant (K_{D}), association rate (kₒₙ) and dissociation rate (k_{off}). Dissociative half-life (t_{1/2}) was calculated from the measured k_{off} using standard equation (t_{1/2}=0.693/k_{off}). The adequateness of the fit was judged by c² values and the randomness of residue distribution.

The SPR binding assay results for Examples 1-31 are provided in Table 3. A geometric mean of binding constant K_{D} was provided when an Example was tested more than once (n is testing replicate number). A blank cell in Table 3 indicates no data was obtained for that Example in that specific assay.

The binding constant K_{D} shows that the exemplified compounds have potent binding capabilities to all KRAS G12C, KRAS G12D, and KRAS G12V receptors, and may be selective over HRAS and NRAS receptors.

TABLE 3
SPR binding assay results

| | | | KRas | | KRas | | KRas | | | | | |
|---|---|---|---|---|---|---|---|---|---|---|---|---|
| Example | KRas SPR | | G12D SPR | | G12C SPR | | G12V SPR | | HRas SPR | | NRas SPR | |
| No | K_{D} (nM) | n | K_{D} (nM) | n | K_{D} (nM) | n | K_{D} (nM) | n | K_{D} (nM) | n | K_{D} (nM) | n |
| 1 | 0.273 | 1 | 0.268 | 1 | 1.23 | 1 | 0.454 | 1 | | | | |
| 2 | 0.422 | 1 | 1.89 | 1 | 1.64 | 1 | 0.515 | 1 | | | | |
| 3 | 4.79 | 7 | 12.3 | 7 | 11.0 | 7 | 10.8 | 7 | >10000 | 1 | >10000 | 1 |
| 4 | 0.308 | 1 | 14.1 | 1 | 1.94 | 1 | 3.69 | 1 | | | | |
| 5 | 2.89 | 1 | 10.5 | 1 | 11.4 | 1 | 22.1 | 1 | | | | |
| 6 | 1.89 | 1 | 2.43 | 1 | 2.67 | 1 | 1.35 | 1 | | | | |
| 7 | 2.55 | 1 | 11.4 | 1 | 8.33 | 1 | 6.71 | 1 | | | | |
| 8 | 0.398 | 1 | 1.69 | 1 | 1.54 | 1 | 11.5 | 1 | | | | |
| 9 | 0.019 | 1 | 0.099 | 1 | 0.447 | 1 | 0.308 | 1 | | | | |
| 10 | 0.030 | 2 | 0.297 | 2 | 0.681 | 2 | 4.86 | 2 | 71.5 | 1 | 346 | 1 |
| 11 | 19.7 | 1 | 23.6 | 1 | 38.9 | 1 | 98.7 | 1 | | | | |
| 12 | 0.022 | 3 | 0.045 | 3 | 1.13 | 3 | 0.117 | 3 | 606 | 1 | 1000 | 1 |
| 13 | 1.25 | 2 | 59.8 | 1 | 7.56 | 1 | 10.4 | 1 | 8920 | 1 | >10000 | 1 |
| 14 | 0.006 | 2 | 0.092 | 2 | 0.263 | 2 | 0.461 | 2 | 198 | 1 | 656 | 1 |
| 15 | 0.118 | 2 | 0.128 | 2 | 0.749 | 2 | 0.274 | 2 | | | | |
| 16 | 5.82 | 2 | 23.8 | 1 | 8.57 | 1 | 5.73 | 1 | >10000 | 1 | >10000 | 1 |
| 17 | 0.362 | 2 | 0.553 | 2 | 0.963 | 2 | 0.269 | 2 | | | | |
| 18 | 0.092 | 1 | 0.214 | 1 | 0.759 | 1 | 0.13 | 1 | | | | |
| 19 | 27.9 | 1 | 78.3 | 1 | 84.7 | 1 | 91.7 | 1 | | | | |
| 20 | 0.075 | 3 | 0.138 | 3 | 0.629 | 3 | 0.098 | 3 | 725 | 1 | 1000 | 1 |
| 21 | 1.76 | 1 | 1.01 | 1 | 1.90 | 1 | 1.46 | 1 | | | | |
| 22 | 11.9 | 1 | 36.9 | 1 | 42.4 | 1 | 46.7 | 1 | | | | |
| 23 | 0.009 | 3 | 0.039 | 3 | 0.019 | 3 | 0.046 | 3 | 473 | 2 | >1000 | 2 |
| 24 | 0.145 | 2 | 0.081 | 2 | 0.079 | 2 | 0.052 | 2 | | | | |
| 25 | 0.001 | 1 | 0.042 | 1 | 0.008 | 1 | 0.149 | 1 | | | | |
| 26 | 0.014 | 2 | 0.046 | 2 | 0.031 | 2 | 0.117 | 2 | | | | |
| 27 | 0.067 | 1 | 0.045 | 1 | 0.020 | 1 | 0.030 | 1 | | | | |
| 28 | 0.061 | 1 | 0.030 | 1 | 0.064 | 1 | 0.028 | 1 | | | | |
| 29 | 5.75 | 2 | 1.55 | 2 | 3.65 | 2 | 1.48 | 2 | | | | |
| 30 | 2.58 | 1 | 0.250 | 1 | 1.10 | 1 | 0.240 | 1 | | | | |
| 31 | 0.194 | 1 | 0.093 | 1 | 0.146 | 1 | 0.183 | 1 | | | | |
| 32 | 0.009 | 1 | 0.004 | 1 | 0.004 | 1 | 0.004 | 1 | | | | |
| 33 | 0.023 | 1 | 0.019 | 1 | 0.019 | 1 | 0.096 | 1 | | | | |

### KRAS Cell Titer Glo (CTG) Assay

The CellTiter-Glo® (CTG) Luminescent Cell Viability Assay is a homogeneous method of determining the number of viable cells in culture based on quantitation of the ATP present, an indicator of metabolically active cells. The CTG is designed for use with multi-well formats, making it ideal for automated high-throughput screening (HTS), cell proliferation and cytotoxicity assays. The homogeneous assay procedure involves adding the single reagent (CellTiter-Glo® Reagent) directly to cells cultured in serum-supplemented medium. Cell washing, removal of medium and multiple pipetting steps are not required. The system detects as few as 15 cells per well in a 384-well format in 10 minutes after adding reagent and mixing.

Cells are grown in humidified 5% CO₂ incubator at 37° C. using the culture conditions outlined below. All cell culture media reagents were purchased from Gibco. Cell lines purchased from ATCC: H358 (non-small cell lung cancer cell line), SW620 (colorectal cancer cell line), PANC08.13 (RPM11640+10% FBS+10 units/ml Insulin, pancreatic cancer cell line). Test and control compounds are dispensed as nanoliter drops according to desired final concentrations in 0.1% DMSO using Echo Acoustic Dispenser onto 384 assay plates (Corning, Cat #3764) prior to cell seeding. Cells were seeded in 40 μL volume per well at the following cell densities (cells per well): H358 (300), SW620 (750), PANC 08.13 (600). Cells are incubated in the presence of compound for 7-days. Viability is determined on Day 7 using CellTiter-Glo® (CTG) Luminescent Cell Viability Assay (Promega). CTG is added to a final volume of 20 μl per well and incubated at room temperature for 15 minutes before luminescence is captured using an EnVision Reader with LUM384 US protocol. Data is analyzed using Activity Base to determine compound response and will be represented either as a percent effect (PCTEFF) or percent control (PCTOCTL) as described: Zero percent effect control (ZPE) (Negative control)−100% DMSO. Hundred percent effect (HPE) (Positive control)−1 uM Trametinib (GSK1120212, MEK inhibitor) (4 nl of 10 mM and 36 nl of DMSO per well). The following equations/nomenclature are used (% Effect; PCTEFF) and (% of Control; PCTOCTL): PCTEFF: 100*(Raw_Data_Value−HPE/ZPE−HPE), PCTOCTL: 100*Raw_Data_Value/User_Defined_Array), where the User_Defined_Array is either summarized HPE or ZPE.

The CTG assay results for some exemplified examples are provided in Table 4. A geometric mean of IC₅₀ (nM) was provided when an Example was tested more than once (n is testing replicate number).

The CTG assay shows that selective exemplified compounds of the present invention have demonstrated anticancer activities for pancreatic cancer, non-small cell lung cancer, and colorectal cancer.

TABLE 4
CTG assay results

| | Panc08.13 | | H358 | | SW620 | |
|---|---|---|---|---|---|---|
| Example | (7-day CTG) | | (7-day CTG) | | (7-day CTG) | |
| No. | IC₅₀ (nM) | n | IC₅₀ (nM) | n | IC₅₀ (nM) | n |
| 9 | 12.3 | 1 | 112 | 1 | 166 | 1 |
| 10 | 8.15 | 2 | 26.9 | 2 | 35.7 | 2 |
| 12 | 19.8 | 2 | 115 | 2 | 136 | 2 |
| 14 | 10.8 | 2 | 41.6 | 2 | 35.3 | 2 |
| 15 | 121 | 1 | 493 | 1 | 871 | 1 |
| 20 | 45.2 | 2 | 142 | 2 | 164 | 2 |
| 23 | 1.2 | 1 | 74 | 2 | 46 | 1 |
| 32 | 2.1 | 4 | 48 | 4 | 45 | 4 |
| 33 | 1.1 | 2 | 3.9 | 2 | 6.6 | 2 |

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

1: A compound of Formula (V): 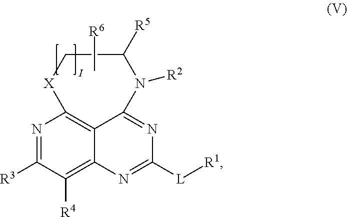
or a pharmaceutically acceptable salt thereof, wherein:
R¹ is selected from the group consisting of: 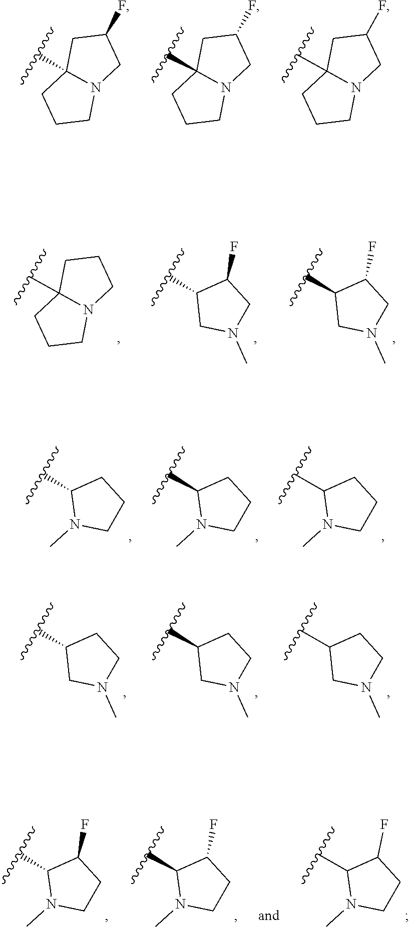
R² is C₁ alkyl, C₃ alkyl, —(C₁ alkylene)-OH, or —(C₃ alkylene)-OH;
R³ is selected from the group consisting of: 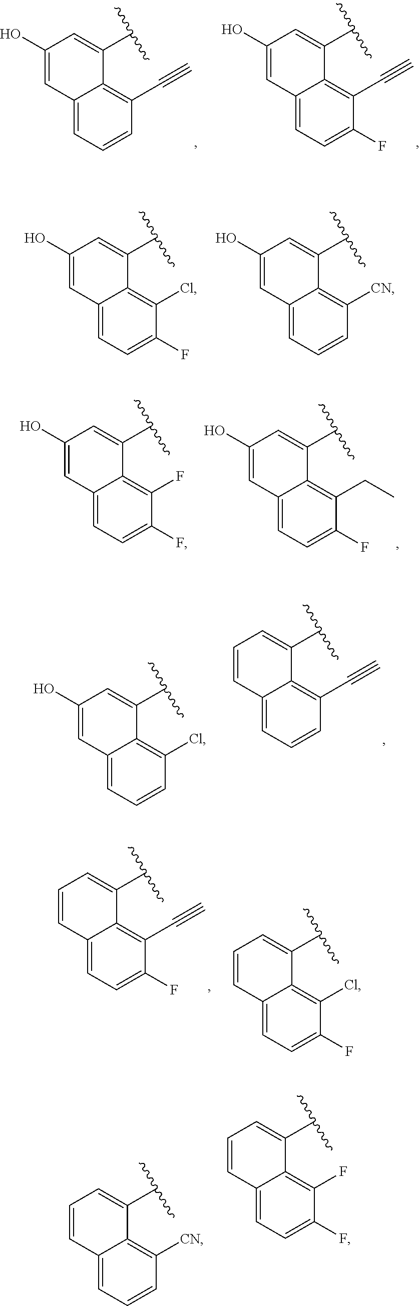 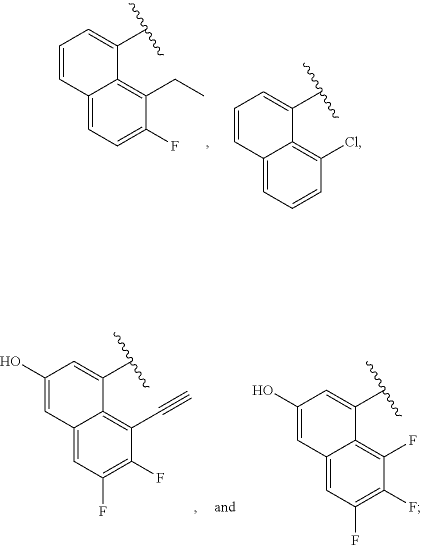
R⁴ is Cl or F;
R⁵ is —(C₁ alkylene)-OH, or C₁ alkyl, or alternatively R² and R⁵ are taken together to form a 7-membered heterocycloalkyl comprising one heteroatom O;
R⁶ represents one or two substituents selected from the group consisting of H, —OH, halogen, —(C₁-C₆ alkylene)-OH, —CN, —(C₁-C₆ alkylene)-CN, C₁-C₆ alkyl, C₃-C₆ cycloalkyl, C₁-C₆ fluoroalkyl, C₃-C₆ fluorocycloalkyl, and C₁-C₆ alkoxy;
L is a linker comprising one, two or three members independently selected from the group consisting of —O—, —S—, —NR⁷—, and —CR⁸R⁹—;
R⁷, R⁸, and R⁹ are each independently H or C₁-C₃ alkyl;
X is O, N, or S; and
l is 1 or 2.

2: The compound of claim 1, or a pharmaceutically acceptable salt thereof, wherein R¹ is selected from the group consisting of:

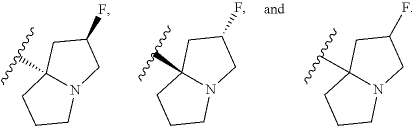

3: The compound of claim 2, or a pharmaceutically acceptable salt thereof, wherein R¹ is:

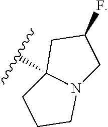 [SMILES: *[C@@]12CCCN1C[C@H](F)C2]

4: The compound of claim 1, or a pharmaceutically acceptable salt thereof, wherein R² is C₃ alkyl, and R⁵ is —(C₁ alkylene)-OH.

5: The compound of claim 1, or a pharmaceutically acceptable salt thereof, wherein R² is —(C₃ alkylene)-OH, and R⁵ is C₁ alkyl.

6: The compound of claim 1, or a pharmaceutically acceptable salt thereof, wherein R² and R⁵ are taken together to form a 7-membered heterocycloalkyl comprising one heteroatom O.

7: The compound of claim 2, or a pharmaceutically acceptable salt thereof, wherein R² and R⁵ are taken together to form a 7-membered heterocycloalkyl comprising one heteroatom O.

8: The compound of claim 1, or a pharmaceutically acceptable salt thereof, wherein R³ is:

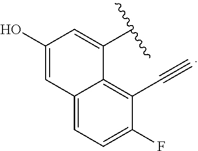 [SMILES: *c1cc(O)cc2ccc(F)c(C#C)c12]

9: The compound of claim 1, or a pharmaceutically acceptable salt thereof, wherein R⁴ is F.

10: The compound of claim 1, or a pharmaceutically acceptable salt thereof, wherein X is O, l is 1.

11: The compound of claim 1, or a pharmaceutically acceptable salt thereof, wherein the compound has Formula (VI):

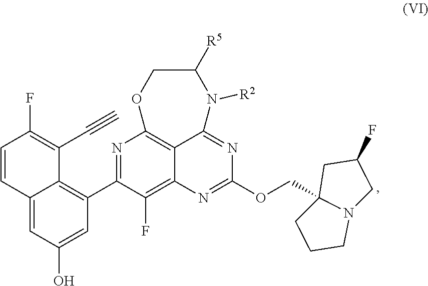

12: The compound of claim 11, or a pharmaceutically acceptable salt thereof, wherein R² is C₃ alkyl, and R⁵ is —(C₁ alkylene)-OH.

13: The compound of claim 11, or a pharmaceutically acceptable salt thereof, wherein R² is —(C₃ alkylene)-OH, and R⁵ is C₁ alkyl.

14: The compound of claim 11, or a pharmaceutically acceptable salt thereof, wherein R² and R⁵ are taken together to form a 7-membered heterocycloalkyl comprising one heteroatom O.

15. (canceled)

16: A compound of Formula (VII) 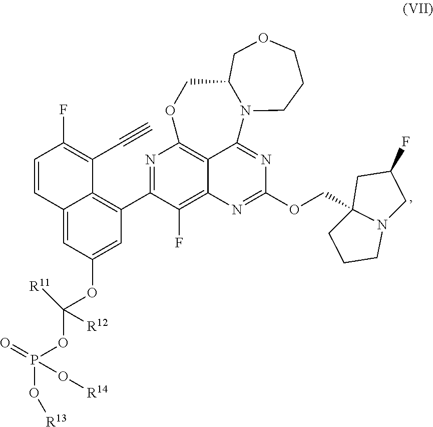
or a pharmaceutically acceptable salt thereof, wherein R¹¹, R¹², R¹³, and R¹⁴ are each independently H or C₁-C₃ alkyl.

17: The compound of claim 16, or a pharmaceutically acceptable salt thereof, wherein R¹¹, R¹², R¹³, and R¹⁴ are each independently H or methyl.

18: The compound of claim 16, or a pharmaceutically acceptable salt thereof, wherein the compound is

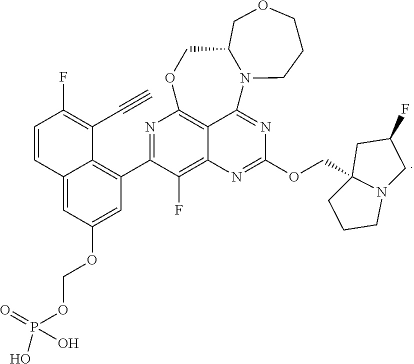 [SMILES: C#Cc1c(F)ccc2cc(OCOP(=O)(O)O)cc(-c3nc4c5c(nc(OC[C@@]67CCCN6C[C@H](F)C7)nc5c3F)N3CCCOC[C@H]3CO4)c12]

19: A compound or a pharmaceutically acceptable salt thereof, selected from the group consisting of:

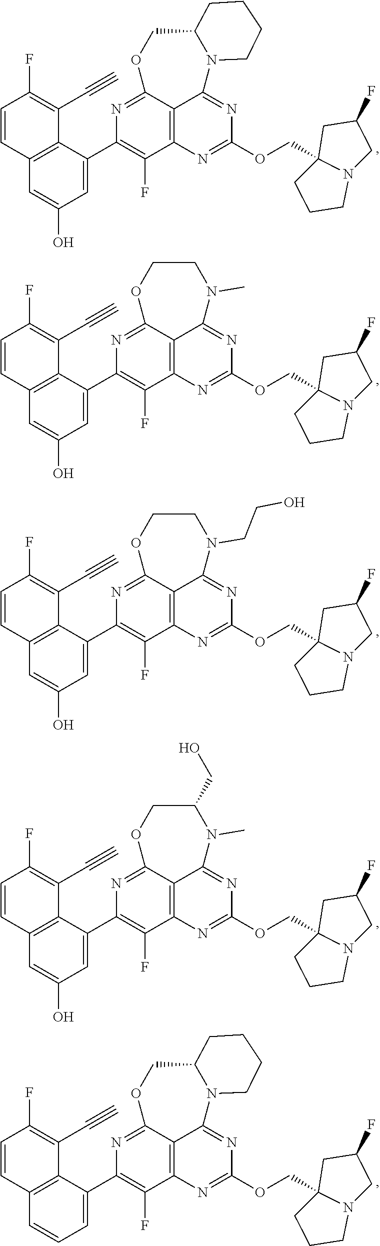 [SMILES: C#Cc1c(F)ccc2cc(O)cc(-c3nc4c5c(nc(OC[C@@]67CCCN6C[C@H](F)C7)nc5c3F)N3CCCC[C@H]3CO4)c12 ; C#Cc1c(F)ccc2cc(O)cc(-c3nc4c5c(nc(OC[C@@]67CCCN6C[C@H](F)C7)nc5c3F)N(C)CCO4)c12 ; C#Cc1c(F)ccc2cc(O)cc(-c3nc4c5c(nc(OC[C@@]67CCCN6C[C@H](F)C7)nc5c3F)N(CCO)CCO4)c12 ; C#Cc1c(F)ccc2cc(O)cc(-c3nc4c5c(nc(OC[C@@]67CCCN6C[C@H](F)C7)nc5c3F)N(C)[C@@H](CO)CO4)c12 ; C#Cc1c(F)ccc2cccc(-c3nc4c5c(nc(OC[C@@]67CCCN6C[C@H](F)C7)nc5c3F)N3CCCC[C@H]3CO4)c12]

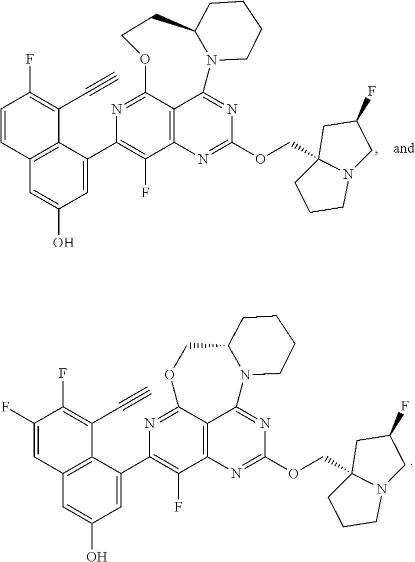

20: A pharmaceutical composition, comprising:
a compound of claim 1, or a pharmaceutically acceptable salt thereof, and
at least one pharmaceutically acceptable excipient.

21: A method for treating cancer, comprising administering to a subject in need thereof a therapeutically effective amount of a compound of claim 1, or a pharmaceutically acceptable salt thereof.

22: A method for treating cancer, comprising administering to a subject in need thereof a therapeutically effective amount of a compound of claim 1, or a pharmaceutically acceptable salt thereof, as a single agent.

23: A method for treating cancer, comprising administering to a subject in need thereof a therapeutically effective amount of a compound of claim 1, or a pharmaceutically acceptable salt thereof, and further comprising administering a therapeutically effective amount of an additional anticancer therapeutic agent.

24: The method for treating cancer of claim 21, wherein the cancer is non-small cell lung cancer (NSCLC), pancreatic cancer, or colorectal cancer.

25-29. (canceled)

30: A method for the treatment of a disorder mediated by inhibition of KRAS G12C, KRAS G12D, and KRAS G12V receptors in a subject, comprising administering to the subject in need thereof a compound of claim 1, or a pharmaceutically acceptable salt thereof, in an amount that is effective for treating the disorder.

31: A pharmaceutical combination comprising a compound of claim 1, or a pharmaceutically acceptable salt thereof, and at least one additional therapeutic agent or a pharmaceutically acceptable salt thereof.

32: A pharmaceutical composition comprising the pharmaceutical combination of claim 31 and at least one excipient.
